(19) United States
(12) Patent Application Publication
(10) Pub. No.: US 2012/0114706 A1
(43) Pub. Date: May 10, 2012

(54) Endorphin Therapy Compositions and Methods of Use Thereof

(76) Inventor: Dipak Kumar Sarkar, Skillman, NJ (US)

(21) Appl. No.: 13/292,709

(22) Filed: Nov. 9, 2011

Related U.S. Application Data
(63) Continuation-in-part of application No. 12/990,896, filed on Dec. 22, 2010.
(60) Provisional application No. 61/051,668, filed on May 8, 2008.
(60) Provisional application No. 61/411,717, filed on Nov. 9, 2010.

(51) Int. Cl. A61K 9/14; A61K 38/17; A61K 35/30; A61P 35/00; A61P 37/00; A61P 29/00; A61P 19/02; A61P 1/00; A61P 37/06; A61P 3/04; A61P 5/00; A61P 3/10; A61P 25/00; A61K 31/7076; B82Y 5/00

(52) U.S. Cl. 424/400; 514/47; 514/17.7; 424/93.7; 514/16.6; 514/5.2; 514/6.9; 977/795; 977/906

(57) ABSTRACT

Methods and compositions for treating cancer and other disorders by β-endorphin therapy are disclosed.

This application is a continuation-in-part of Ser. No. 12/990,896, filed on Dec. 22, 2010, which is a §371 application of PCT/US2009/002894, filed on May 8, 2009, which claims priority under 35 U.S.C. §119(e) to U.S. Provisional Patent Application No. 61/051,668, filed on May 8, 2008. The instant application also claims priority under 35 U.S.C. §119(e) to U.S. Provisional Patent Application No. 61/411,717, filed on Nov. 9, 2010. The foregoing applications are incorporated by reference herein.

This invention was made with government support under R01 AA015718 awarded by the National Institutes of Health. The government has certain rights in the invention.

### FIELD OF THE INVENTION

The present invention relates to compositions and methods to increase the body's innate immunity to prevent tumor cell growth and immune-related diseases. More specifically, the present invention relates to beta-endorphin compositions and methods to increase innate immunity, treat/inhibit cancer, and treat/inhibit anxiety and anxiety-like disorders.

### BACKGROUND OF THE INVENTION

Several publications and patent documents are cited throughout the specification in order to describe the state of the art to which this invention pertains. Each of these citations is incorporated herein by reference as though set forth in full.

The hypothalamus consists of several groups of hormone-secreting neurons that are critical for various neuroendocrine functions (Settle, M. (2000) Neonatal Netw., 19:9-14). Most of the neurons in the hypothalamus are derived from the proliferative neuroepithelium of the third ventricle (van Eerdenburg et al. (1994) Brain Res. Dev. Brain Res., 79:290-296). A study of cell development in the rat hypothalamus, using [³H]thymidine uptake assays, revealed that most of the neurons of the tuberomammillary and arcuate nuclei have late-forming starts, beginning after embryonic day 16 and continuing until birth (Altman et al. (1978) J. Comp. Neurol., 182:995-1015). However, the inductive signal involved in the generation of specific neuronal cell types from these embryonic cells has not been identified. By using cells from the hypothalamus of rat embryos, it has been shown that cAMP-elevating agents protect against ethanol-induced death of β-endorphin (BEP) neurons (De et al. (1994) J. Biol. Chem., 269:26697-26705). These findings have raised the question of whether this neurotrophic factor can be used to direct heterologous sets of neural stem cells (NSCs) into specific neuronal phenotype.

### SUMMARY OF THE INVENTION

A novel method to isolate neural stem cells (NSC) from fetal hypothalamus has been developed. The methods include preparation of dissociated cells from fetal rat hypothalami. Then, purifying these mixed neuronal, glial and stem cells from mixed neural and neural progenitor cells by the use of uridine and fluodeoxyuridine to kill the glial cells and leaving the live neural and neural stem cells in cultures. After selection, the method comprises growing these cells for several generations in cultures so that only the neural stem cells remain in cultures. Once this is achieved, the cells are maintained in cultures in the presence of stem cell medium with lymphokine inhibiting factor (LIF; about 0.1 micro gram/ml) and basic fibroblast growth factor (bFGF; about 20 ng/ml) so that only neural stem cells with the ability to differentiate into beta-endorphin (BEP) neurons remain in cultures.

A method to differentiate beta-endorphin neuronal cells from neural stem cells has also been developed. Neural stem cells can be differentiated to beta-endorphin neurons if they were removed from the influence of LIF and then maintaining them in the environment favoring the survival of neurons (e.g., neuron culture media) and then treating them for about 1 week with pituitary adenylate cyclase activating peptide (PACAP) and dibutyryl cyclic adenylate cyclase (dbcAMP). Beta-endorphin neuron purity increases when neural stem cells were treated with both of these agents at about 10 micromolar dose/each but other doses are also effective at different efficiency. Treatment with only dbcAMP or PACAP is also effective but with less efficiency. Application of dbcAMP or cAMP activating agent is also effective to differentiate endogenous neural stem cells if these agents are delivered to the brain via a delivery system like nanospheres or other vehicles.

It has been discovered that beta-endorphin cells inhibit body stress responses, activate natural killer (NK) cells and inhibit proinflammatory cytokines. NK cells are mediators of the innate immune response critical for defense against infectious viral and bacterial diseases (e.g. AIDS, etc.) and cancers (e.g., prostate, breast, etc.). Increases in innate immunity by beta-endorphin cells provide a unique approach to combat cancer and cancer metastasis, various immune diseases, and pathogenic infections. Reduction of inflammatory cytokines not only prevents tumor growth and progression but also reduces other diseases associated with inflammations such as rheumatoid arthritis development. Additionally, these cells suppress stress axis function and thereby are beneficial in stress reduction and in controlling stress-induced metabolic diseases.

Market Applications: Therapeutics, Cancers (prostate, breast and other cancers), Tumor Metastasis, Infectious Diseases, Stress Control, Metabolic Diseases, Fetal alcohol patients stress and immune related problems.

Advantages: Studies show the ability of the in vitro produced beta-endorphin cells maintain functionality in vivo and increase NK cytolytic activity. Initial pre-clinical models of prostate and breast cancers demonstrate the potential for enhancing innate immunity to prevent cancer growth and progression and metastatic invasion. In preclinical model of stress control identify a significant beneficial effect of beta-endorphin cell therapy in stress reduction in fetal alcohol exposed subjects. Furthermore, beta-endorphin cells showed unique ability to reduce the incidence of rheumatoid arthritis.

In certain embodiments, the invention is directed to a method of isolating neural stem cells from a fetal hypothalamus comprising: isolating mixed neural and neural stem cells from glial cells in a fetal hypothalamus, and growing the isolated mixed neural and neural stem cells for several generations in cultures so that only the neural stem cells remain in the cultures. In certain other embodiments, the method further comprises introducing an agent that kills the glial cells but not the neural and neural stem cells in cultures. In certain other embodiments, the agent is selected from the group consisting of uridine, fluodeoxyuridine and a combination thereof.

In accordance with any of the above embodiments, the invention further comprises maintaining the neural stem cells in cultures in the presence of stem cell medium with lymphokine inhibiting factor (LIF) so that only neural stem cells with the ability to differentiate into beta-endorphin (BEP) neurons remain in cultures. In certain other embodiments, the concentration of LIF is about 0.1 microgram/ml. In certain other embodiments, the cultures also contain basic fibroblast growth factor (bFGF). In certain other embodiments, the concentration of bFGF is about 20 ng/ml.

In accordance with any of the above embodiments, the invention is further directed to a method of differentiating beta-endorphin neuronal cells from neural stem cells that are under the influence of LIF comprising: (i) removing the influence of LIF from the neural stem cells, (ii) maintaining the neural stem cells in an environment favoring the survival of neurons, and then (iii) treating the neural stem cells with a differentiating agent selected from group consisting of (a) pituitary adenylate cyclase activating peptide (PACAP), (b) dibutyryl cyclic adenylate cyclase (dbcAMP) and (c) a combination thereof. In certain embodiments, the environment in step (ii) is neuron culture media. In certain other embodiments, the treating in step (iii) is performed for about 7 days. In certain other embodiments, the agent in step (iii) is a combination of PACAP and dbcAMP. In still other embodiments, the dose of the agent is about a 10 micromolar dose. In certain other embodiments, the dose of PACAP is about a 10 micromolar dose and the dose of dbcAMP is about a 10 micromolar dose.

The invention is also directed to a method of differentiating endogenous neural stem cells into BEP cells in a patient in need thereof comprising: (i) administering an effective amount of an agent selected from the group consisting of (a) pituitary adenylate cyclase activating peptide (PACAP), (b) dibutyryl cyclic adenylate cyclase (dbcAMP) and (c) a combination thereof into the central nervous system. In certain embodiments, the agent is administered into the brain. In certain other embodiments, the agent is administered into the hypothalamus. In certain other embodiments, the agent is administered into the third ventral. In accordance with any of the above embodiments, the invention is also directed to a method wherein the agent is administered as a pharmaceutically acceptable nanosphere.

In accordance with any of the above embodiments, the invention also provides a method wherein the amount of agent administered is sufficient to provide BEP cell differentiation to reduce physiological stress responses. In certain other embodiments, the amount of agent administered is sufficient to provide BEP differentiation to activate natural killer (NK) cells and inhibit proinflammatory cytokines. In certain other embodiments, the amount of agent administered is sufficient to improve the innate immune response critical for defense against diseases selected from the group consisting of infectious diseases including viral and bacterial diseases, and hyperproliferative diseases such as cancers.

In accordance with any of the above embodiments, the invention is also directed to a method wherein the disease is neoplasia. In certain other embodiments, the disease is prostate cancer. In certain other embodiments, the disease is breast cancer. In still other embodiments, the disease is metastatic breast cancer.

In certain other embodiments, the amount of agent is administered is sufficient to provide BEP cell differentiation to reduce inflammation associated with immunologic diseases. In certain other embodiments, the immunological disease is selected from the group consisting of rheumatoid arthritis, adult onset diabetes (type II), obesity, thyroid disorder, celiac disease, inflammatory bowel syndrome, lupus and a combination thereof.

In accordance with the instant invention, compositions and method for treating, inhibiting (reducing), and/or preventing cancer, immunological disease, pathogenic infections, and/or inflammation in a subject in need thereof are provided. In a particular embodiment, the methods comprise administering at least one agent which increases β-endorphin. In certain embodiments, the method comprises administering β-endorphin neurons to the subject.

In accordance with another aspect of the instant invention, compositions and method for treating, inhibiting, and/or preventing anxiety and anxiety/stress-related disorders in a patient in need thereof are provided. In a particular embodiment, the methods comprise administering at least one agent which increases β-endorphin. In certain embodiments, the method comprises administering β-endorphin neurons to the subject.

### BRIEF DESCRIPTION OF THE DRAWINGS

FIG. 1 is an image of neuronal stem cells and provides characterization of hypothalamic neuronal stem cells in cultures. FIGS. 1A-1C: Phase-contrast images of embryonic rat hypothalamic neuronal stem cells at the stage of aggregated primary sphere (FIG. 1A) and single (FIG. 1B) and aggregated (FIG. 1C) secondary spheres. FIG. 1D: Immunofluorescence staining of nestin for primary single spheres (small arrows) or aggregated spheres (large arrows). Secondary spheres also stained for nestin (FIG. 1E) or vimentin (FIG. 1F). “-”=20 μm.

FIG. 2 is an image of neuronal stem cells and shows the characterization of hypothalamic neuronal stem cells at various phases of differentiation by PACAP and cAMP in cultures. FIG. 2A: Phase-contrast images of neuronal stem cells treated with 10 μM of PACAP and 10 μM of cAMP for a period of 3 days. FIGS. 2B and 2C: Representative photograph showing the immunofluorescence staining for vimentin (FIG. 2B) and α-internexin (FIG. 2C) in the early phase of differentiation at 3 d. “-”=20 μm. FIG. 2D: Phase-contrast images of neuronal stem cells treated with 10 μM of PACAP and 10 μM of cAMP for a period of 1 week. FIG. 2E: A representative photograph showing the immunofluorescence staining for the neuronal marker NF-M. FIG. 2F: Immunofluorescence staining for BEP shown. FIG. 2G: Phase-contrast images of control neuronal stem cells maintained without the PACAP and cAMP in neuronal culture medium. FIG. 2H: No staining was seen when these control undifferentiated neuronal stem cells were stained for BEP. “-”=20 μm.

FIG. 3 is an image of neuronal stem cells and shows the characterization of PACAP- and cAMP-induced differentiated hypothalamic neuronal stem cells in cultures. FIG. 3A: Phase-contrast images of neuronal stem cells treated with 10 μM of PACAP and 10 μM of cAMP for a period of 1 week and then maintained in serum-free defined neuron culture media without the differentiation factors for a period of 1 week. FIGS. 3B and 3C: A representative photograph showing the immunofluorescence staining for neuronal markers MAP2 (FIG. 3B) and type III β-tubulin (FIG. 3C). FIG. 3D: No staining was seen when these cells were stained for a glial marker GFAP. FIG. 3E: Immunofluorescence staining for BEP shown. FIG. 3F: BEP staining was absent when cells were stained with the BEP antibody that was preincubated with excess antigen. The staining in this figure represents the DAPI staining for the nucleus of the cells in culture. “-”=20 μm.

FIG. 4 is an image of neuronal stem cells and shows the characterization of neuropeptide production by the PACAP- and cAMP-induced differentiated hypothalamic neuronal stem cells in cultures. Representative photographs showing the immunofluorescence staining for various cAMP-responsive neuropeptides (D-endorphin, BEP; neuropeptide y, NPY; gonadotropin releasing hormone, GnRH; tyrosine hydroxylase, TH). The staining represents DAPI staining for the nucleus of the cells. Note that the differentiated NSC gave the combined staining for BEP and the cell nucleus. “-”=20 μm.

FIG. 5 is a graphical depiction showing characterization of the basal and PgE1-induced increase in BEP release and POMC mRNA expression from hypothalamic neuronal stem cells after PACAP- and cAMP-induced differentiation. FIGS. 5A and 5B: Shows the time characteristic of the BEP release (FIG. 5A) and POMC mRNA expression (FIG. 5B) from neuronal stem cells differentiated in the presence of PACAP (10 μM) and dbcAMP (10 μM). No BEP was detected in media samples collected from control cultures without the PACAP and dbcAMP treatment (O-day) or the cultures treated with the differentiating agents for 3-day. P<0.05, significantly different from the values of the rest of the groups. FIGS. 5C and 5D: Demonstrates the dose-response and synergistic effects of PACAP and dbcAMP on BEP release (FIG. 5C) and POMC mRNA expression (FIG. 5D) from differentiated neuronal stem cells treated with the drugs for 1 week and then without the drugs for 1 week. The control group was treated similarly with vehicle. P<0.05, significantly different from the values of the 1-μM dose of the similar agent. P<0.05, significantly different from the values of the rest of the groups. FIGS. 5E 5F: Shows the PgE1 (10 μM)-induced BEP release response (FIG. 5E) and POMC mRNA expression response (FIG. 5F) from differentiated neuronal stem cells treated as in FIG. 5A. Values are presented as a percentage of vehicle-treated control. P<0.05, significantly different from the values of control. P<0.05, significantly different from the values at 1 week.

FIGS. 6 A-D is an image of neuronal stem cells and shows the determination of in vivo functionality of the PACAP and dbcAMP-induced differentiated hypothalamic neuronal stem cells. FIGS. 6A-6D: Immunocytochemical characterization of differentiated neuronal stem cells in the presence of PACAP (10 μM) and dbcAMP (10 μM) at 2 weeks after transplantation into the PVN of the hypothalamus in male rats. Representative photographs showing BrdU-stained cells at the site of transplantation (FIG. 6A). “-”=10 μM. High-power view of a photograph showing immunofluorescence staining for BrdU and BEP of transplanted cells (FIG. 6B). Many BEP-stained cells show long processes (arrows; FIG. 5C) “-”=20 μM. Merged images from FIG. 6B and FIG. 6C show numerous BEP- and BrdU-double stained cells (FIG. 6D). FIGS. 6E-6G are a graphical depiction showing in vivo functionality of the PACAP and dbcAMP-induced differentiated hypothalamic neuronal stem cells. FIG. 6E: POMC mRNA levels in the lobe of PVN with transplanted differentiated neuronal stem cells (TP) and in the contralateral lobe of the PVN that underwent sham-transplant surgery (S-TP). P<0.05, significantly different from the values of S-TP. FIGS. 6F-6G: Physiological responses of transplanted cells. POMC mRNA (FIG. 6F) and CRH mRNA (FIG. 6G) levels in the PVN lobe with TP and in the contralateral lobe of the PVN with S-TP after i.p. administration of LPS or of saline in pubertal male rats exposed to alcohol in fetal life (FAE). The effect of LPS or saline treatment in pubertal male rats exposed to no alcohol in fetal life (control) was shown for comparison. N=6-8. a, P<0.05, vs. saline. P<0.05, vs. LPS-treated control or S-TP animals.

FIG. 7 is a graphical depiction of a determination of the effect of NSC-BEP transplants on NK cell cytolytic function. Adult male rats (90 days old) fed during embryonic days 11 through 21 via dams; with alcohol (alcohol-fed rats), an isocaloric liquid diet (pair-fed rats) or with a regular diet (ad lib-fed rats) were transplanted with NSC-BEP (BEP; 20,000 cells/1 μl) or cortical cells (CORT; 20,000 cells/1 μl) into the left PVN. After 3 weeks rats were sacrificed and the spleens were collected. Splenocytes were prepared and assayed for NK cell cytolytic activity against YAC-lymphoma cells by a standard 4 hour chromium-51 release cytolytic assay. The histograms represent mean±SEM of NK activity in lytic units (LU). N=5-7 rats. P<0.05, vs. ad lib and pair-fed rats. P<0.05, vs. CORT cell-treated or untreated animals fed similarly during embryonic life.

FIG. 8 is a graphical depiction of a determination of the effect of NSC-BEP transplants on NK cell functions: Dose-dependent effect. Adult male rats (90 days old) alcohol-fed, pair-fed or ad lib-fed during the prenatal period were transplanted with NSC-BEP or cortical cells into one PVN (×1) or two PVN (×2). After 3 weeks, approximately 1 ml of blood was collected from the orbital sinus of each rat and used for PBMC preparation and plasma separation. PBMC samples were used to determine the NK cytolytic activity by a standard 4 hour chromium-51 release cytolytic assay. Plasma samples were used to determine IFN-gamma and TNF-alpha levels by ELISA. Due to low blood volumes in some samples, TNF-alpha could not be measured in these samples. The histograms represent mean±SEM values from 5-7 rats. P<0.05, vs. CORT-cell transplant in rats that were similarly treated prenatally. P<0.05, vs. BEP×1-cell transplant in rats that were similarly treated prenatally. P<0.05, compared to the rest of the groups.

FIG. 9 is a graphical depiction of a determination that NSC-BEP cell transplants in the paraventricular nuclei of the hypothalamus reduce the ability of carcinogen and hormone to induce prostate tumors. Treatments with NMU and testosterone increased the weight of prostate (a crude measure of tumor in prostate) about 3-fold in pair-fed and ad lib fed rats transplanted with control cells (cortical cells were used as control cells in pair-fed rats, and neuronal stem cells without differentiation were used as control cells in ad lib fed rats; CONT-TP) in the PVNs. Transplantation of NSC-BEP in both PVNs (NSC-BEP-TP) suppressed the ability of carcinogen and hormone to increase prostate weight. P<0.01, significantly different from the control treatments within the similarly-fed group.

FIG. 10 is an image of histopathology of prostate of rats transplanted with control cortical cells, non-differentiated NSC cells or NSC-BEP cells. The treatment of NMU and testosterone induced significant neoplasia in prostates of rats transplanted with control cortical (top row; showing hyperplasia and adenocarcinoma) or NSC cells (bottom left; showing adenocarcinoma). Prostate histology appears to be mostly normal in NSC-BEP transplanted rats (a representative picture is shown on the bottom right). Magnifications are in 5× except the bottom right, which is in 2×.

FIG. 11 is an image of cancer prostate tissue in evaluation of the effect of BEP cell transplants on the MNU and testosterone-induced prostate cancers. Adult male rats were transplanted with in vitro differentiated BEP cells or cortical cells (CONT) bilaterally in the PVN of male rats. These rats were then treated with MNU and testosterone treatments and used for determination of histopathology of prostates. FIGS. 11A-11C: Prostates of rats transplanted with CONT showed lesions ranging from epithelial hyperplasia with mild atypia (FIG. 11A) to high-grade PIN (FIG. 11B) and occasionally invasive adenocarcinoma (shown by arrows; FIG. 11C). FIGS. 11D-11E: Prostates of rats transplanted with BEP cells demonstrated very mild changes, ranging from minimal (FIG. 11D) to moderate hyperplasia (shown by arrows; FIG. 11E). “-”=10 μm.

FIGS. 12A and 12B provide graphs representing male Fischer rats at age of 50 days old that were implanted with β-EP neuronal cells (20,000 cells/μl) in both sides of PVN. After 6 months, animals were lightly anesthetized with isoflurane, and vein blood was collected by orbital puncture. In LPS treated group, animals were i.p. injected with LPS (100 μg/kg body weight), and blood was collected after 2 hours by orbital puncture. Plasma hormone levels were measured using ELISA. β-EP implanted animals had lower corticosterone and α-MSH levels comparing to normal animals under LPS treatment. **, p<0.01, comparing to control.

FIG. 13 shows the physiological responses of transplanted cells in the PVN. Male rats (35- to 40-day old) were exposed to alcohol during embryonic days 11 through 21 with alcohol (AF) or isocaloric liquid diet (PF) were transplanted with BEP cells (20,000 cells/1 μl) into the left PVN or nonviable BEP cells (CONT; 20,000 cells/1 μl) into the right PVN. After 2 weeks, rats underwent LPS or saline treatment and then were sacrificed after 3 hours. PVN tissues of these rats were collected and used for CRH measurements. CRH mRNA levels with LPS treatment decreased back to normal in AF rats after BEP implantation. n=6. c, p<0.01 comparing to PF+BEP+LPS.

FIGS. 14A and 14B show the determination of the viability of β-EP cells transplanted in the PVN. Female SD rats were transplanted with β-EP cells at 50 days old. After 13 weeks, animals were sacrificed and brains were sectioned and stained with β-EP antibody. Pictures show that clusters of β-EP cells remained in the PVN area (A: 20×, B: 40×).

FIGS. 15A-15D show the evaluation of the effects of BEP cell transplants on immune functions. Sprague Dawley rats were transplanted with β-EP (BEP) cells or cortical cells (CONT) into one PVN (+) or both sides of PVN (++) for 4 weeks. Shown are dose-response effects of BEP cell transplants on splenic NK cell cytolytic activity (FIG. 15A), PBMC-derived NK cell cytolytic activity (FIG. 15B), plasma IFN-γ (FIG. 15C), and plasma TNF-α (FIG. 15D). n=5-7 rats. a, P<0.05; b, P<0.01; c, P<0.001 vs. the CONT.

FIGS. 16A-16C show a cell population investigation using flow cytometry. Cells were fixed in PFA and stained with FITC-linked antibodies. After NK cell purification with MACS, NK-positive cells were enriched to about 70% in final cell population (FIG. 16A). After T cell purification, a cell population with about 70% of CD4+ cells and 30% CD8+ were collected.

FIGS. 17A and 17B show the cytolytic activity of enriched NK cells from PBMCs. Breast cancer cell line MADB106 cells were used as target cells. 3 different ratios of effecter to target cell ratios (E:T ratio) were used (10:1, 5:1 and 2.5:1). Without LPS treatment, there isn't significant difference between β-EP animals and control animals (FIG. 17A). However, 2 hours after LPS treatment (100 μg/kg body weight), β-EP animals showed significantly higher NK cell cytolytic activity (FIG. 17B). ***, p<0.001, comparing to control. *, p<0.05, comparing to control.

FIGS. 18A and 18B show monocyte migration activity in PBMCs. Blood were collected by orbital puncture. PBMCs were separated from blood by spinning on histopaque-1083. Macrophage migration activity were detected using CytoSelect™ 96-well cell migration assay (5 μm), which has a filter pore size specific for macrophage migration. Both without LPS treatment (FIG. 18A) and with LPS treatment (FIG. 18B), β-EP transplanted animals have higher macrophage migration activity in PBMCs. ***, p<0.001, comparing to control.

FIG. 19 shows β-EP transplanted animals have significantly lower average tumor number (FIG. 19A, tumor number per rat), tumor volume (FIG. 19B, total tumor volume/animal number), and tumor incidence (FIG. 19C, ratio of animal who had tumor). Histology diagnosis also showed that β-EP animals had more benign tumor comparing to cortical cell transplanted animals (FIG. 19D). FIGS. 19E and 19F show examples of pictures of benign tumor (adenoma) and malignant tumor (adenocarcinoma). **, p<0.01 comparing to CC; ***, p<0.001, comparing to CC; n=16. Analyzed by two-way ANOVA.

FIG. 20 provides examples of tumors developed in CC group.

FIG. 21 shows naltrexone+DPDPE treated animals had slightly decreased average tumor number (FIG. 21A) and tumor incidence (FIG. 21C), but not significantly lower tumor volume (FIG. 21B). *, p<0.05, n=9, analyzed by two-way ANOVA.

FIG. 22 provides representative pictures of different appearance of tumor developed after MADB106 cells inoculation. FIG. 22A: tumor growing on the neck. FIG. 22B: generally normal lung with a granule of tumor (arrow). FIG. 22C: lung covered with granules of tumor. FIG. 22D: tumor mass with blood and necrosis.

FIG. 23 provides representative pictures of cortical cell transplanted animals with lung-metastasis and β-EP transplanted animals without tumor in both lung and neck. FIG. 23A: lung of β-EP animals which are free of tumor, and control animals with tumor. FIG. 23B: Animals anesthetized showing tumor on the neck.

FIG. 24 shows immune activities in Fischer male rats with and without β-EP transplantation after tumor cell inoculation. β-EP animals showed higher macrophage migration activity (FIG. 24A), NK cell percentage in PBMC (FIG. 24B), increased some pro-inflammatory cytokines, and decreased the other pro-inflammatory cytokines in plasma and spleen (FIGS. 24C, 24D). *, p<0.05; **, p<0.01; ***; p<0.001; comparing to control.

FIG. 25A shows β-EP neuron numbers in hypothalamus of animals implanted with different types of nanospheres containing: (1) plain nanosphere (control), (2) 10 μM cAMP, (3) 10 μM PACAP, or (4) 5 μM cAMP+5 μM PACAP. It was shown that brains implanted with 10 μM cAMP has significantly increased number of β-EP neurons, while brains implanted with the same dose of PACAP or half dose of cAMP+PACAP do not have change in β-EP neuron numbers. *, p<0.05, comparing to control. FIG. 16B shows female Sprague Dawley rats were implanted with nanospheres containing cAMP, cAMP+PACAP, or control nanospheres at 2˜3 months old. After 4 months, animals were sacrificed, and hypothalamus tissues were homogenized. β-EP ELISA and protein assay were used to determine β-EP and protein concentration. It was found that nanosphere containing cAMP or cAMP+PACAP increased β-EP to total protein ratio in the hypothalamus. *, p<0.05, comparing to control. n=5.

FIG. 26 shows adult female Fischer rats were implanted with nanospheres containing 10 μM cAMP or 5 μM cAMP+5 μM PACAP, or plain nanospheres into 3rd ventricle. 2 weeks later, half of the animals were i.p. injected with naloxone (1 mg/kg body weight), which is aμ-opioid receptor antagonist, specifically blocking the effect of β-EP. 12 hours later, animals were anesthetized with Nembutal, and blood was drawn from jugular vein. Plasma corticosterone level was measured with ELISA. It was found that implantation of cAMP or cAMP+PACAP-delivering nanospheres decreased plasma level of corticosterone, and that administration of naloxone blocked the effect of nanospheres, probably by blocking the effect of β-EP. **, p<0.01, comparing to control animal without naloxone. n=5.

FIG. 27 shows fetal alcohol exposed Sprague Dawley rats were implanted with 1 μl control or cAMP-containing nanospheres into 3rd ventricle at 2 days old, using a 5 μl Hamilton syringe. 2 months later, animals were anesthetized, and 1 ml of blood was drawn from jugular vein. Plasma corticosterone level was measured by ELISA. cAMP-delivering nanospheres significantly decreased corticosterone levels in both male and female fetal alcohol exposed animals. *, p<0.05, comparing to AF control. n=6.

FIG. 28 shows cAMP nanosphere implanted animals have significantly lower average tumor number (FIG. 28A, tumor number per rat), tumor volume (FIG. 28B, total tumor volume/animal number), and tumor incidence (FIG. 28C, ratio of animal who had tumor). Histology diagnosis also showed that cAMP nanosphere implanted animals had more benign tumor comparing to control nanosphere and PACAP nanosphere implanted animals (FIG. 28D). FIGS. 28E and 28F provide examples of nanospheres with fluorescence inside the third ventricle. *, p<0.05, **, p<0.01, ***, p<0.001, comparing to CC; n=16. Data analyzed by two-way ANOVA.

FIG. 29 shows the effect of cAMP-delivering nanospheres on MADB106 cell metastasis. Both cAMP and cAMP+PACAP nanospheres reduced the chance of lung tumor, while naloxone treatment before and after tumor inoculation reversed the effect. n=5.

FIG. 30 shows NK cells cytolytic activity 24 hours after tumor inoculation (FIG. 30A); T cells cytolytic activity 7 days after tumor cells inoculation (FIG. 30B); PBMC migration assay 24 hours and 7 days after tumor inoculation (FIG. 30D); and final tumor observed in lung and neck at sacrifice (FIG. 30E).

FIG. 31 shows the effect of anxiogenic and anxiolytic agents, and CC and β-EP neuron transplants on anxiety-like behaviors in the EPM. All groups were compared using one-way ANOVA followed by Newman-Keuls Multiple Comparison Test. CC vs. β-EP, DZP+ vs. YOH−: *, p<0.05, **, p<0.01, ***, p<0.001. DZP+ vs. β-EP: a, p<0.05.

FIG. 32 shows the effect of CC and β-EP neuron transplants on anxiety-like behaviors in the OF. All groups were compared using one-way ANOVA followed by Newman-Keuls Multiple Comparison Test. CC vs. β-EP: *, p<0.05, ***, p<0.001.

FIG. 33 A shows the effect of CC and β-EP neuron transplants on basal corticosterone levels. Student's t-test revealed significantly reduced basal levels of CORT in β-EP rats, (*, p<0.05). FIG. 33B shows the effect of CC and β-EP neuron transplants on corticosterone response during and following restraint stress (60 minutes). Two-way ANOVA, with factors TIME and GROUP, revealed significant main effects of time and group, and interaction. Bonferroni post hoc tests revealed significantly reduced CORT levels at peak times, * p<0.05; ***, p<0.001.

FIG. 34 represents macroscopic appearance of representative liver from cortical cells (FIGS. 34A-34C) and BEP neurons implanted group (FIG. 34D). Group 2 rat liver showed cirrhotic liver white mass (arrows) in (FIG. 34A) and arrows indicates a large tumor of hepatocellular carcinoma (FIGS. 34B and 34C). FIG. 34D: Representative liver from group 4 (BEP neurons implanted) show no gross tumor or abnormalities. No tumor was found in vehicle control groups 1 and 3 either.

FIG. 35 provides the histopathology of liver from cortical cell implanted groups showing various abnormalities (FIGS. 35A-35C). Appearance of fibrosis with deposition of fibrosis septae, extended between vascular structures (FIG. 35A), extensive collapse and increased deposition of reticulum and development of extensive fibrosis in centrilobular areas (FIG. 35B), well differentiated HCCs with compressed hepatic parenchyma (FIG. 35C). Represents liver section from BEP neurons implanted animal showing almost normal liver morphology with mild fibrosis septae (FIG. 35D).

FIG. 36 provides histologic analyses of liver fibrosis. Assessment of liver fibrosis was performed using Sirius Red (FIGS. 36A-36C) and Massson's trichrome staining (FIGS. 36D-36F). Fibrosis was severe and surrounds the centrilobular vein and creating a fine network of fibers (FIGS. 36A, 36B, 36D and 36E) in cortical cells implanted groups, whereas accumulation of collagen was very normal in the BEP neuronal transplantation groups (FIGS. 36C and 36F).

FIG. 37 provides immunohistochemical analyses of placental glutathione S-transferase (GST-pi). Immunodetection of GST-pi in the liver of cortical cell-implanted groups showed a high number of positive preneoplastic foci (FIGS. 37A and 37B) and few small foci in BEP neuronal transplantation groups (FIGS. 37C and 37D).

FIG. 38 shows BEP neuronal transplant in the hypothalamus suppresses mammary tumor growth and progression. Animals were administered with a single i.p. injection of NMU at 50 mg/kg at 50 days of age. After 6 weeks of NMU, rats were administered with 20,000 cells/mL of BEP cells or cortical cells (control) into the PVN bilaterally. Rats were palpated for tumors once a week following injections. FIG. 38A: a representative photomicrograph showing BEP cells in primary cultures after staining for the BEP peptide by immunofluorescence technique. FIG. 38B: a representative immunofluorescence pictures of BEP cells in the PVN area of the hypothalamus after 6 months of transplants. FIG. 38C: corticosterone response to LPS. FIG. 38D: body weight changes. FIG. 38E: graph represents percent (tumor incidence) of rats presenting with tumors each week postinjection. FIG. 38F: graph represents average number of tumors per treatment group each week postinjection. FIG. 38G: average volume of tumor per animal in each group. FIG. 38H: tumors malignancy rate as determined by histologic evaluation. Percentage of each histologic tumor type that developed per treatment group. Data of FIG. 38C were analyzed using 1-way ANOVA followed by Newman-Keuls post-hoc analysis. Data in FIGS. 38D-38G were analyzed using a 2-way NOVA and only the data shown in FIGS. 38E-38G were found to have significant treatment and time interactions at P<0.001 (n=12/group), and FIG. 38F was analyzed using a χ² test with a P<0.0001. FIGS. 38I-38L: representative images of different histologic tumor types.

FIG. 39 shows the effects of BEP neuron transplants on the expression of inflammatory markers (TNF-α and NF-κB), EMT factors (Snail, Slug, Twist), mesenchymal marker (N-cadherin), and epithelial marker (E-cadherin) in tumor tissues. Cortical cells were used as control transplants. Tumors were removed from animals treated with MNU followed by BEP neuron or control neuron transplants as described in FIG. 38 legends. Cellular levels of inflammatory and EMT factors were determined by immunohistochemistry (shown on the left) and Western blotting (shown on the right in each panel). FIG. 39A: TNF-αexpression. FIG. 39B: NF-κB expression. FIG. 39C: Snail expression. FIG. 39D: Slug expression. FIG. 39E: Twist expression. FIG. 39F: N-cadherin expression. FIG. 39G: E-cadherin expression. Actin expression served as an internal control. n=5 rats. ***, P<0.001 versus control.

FIG. 40 shows the evaluation of effects of BEP-cell transplantation on mammary cancer metastasis. Rats were transplanted with 20,000 viable cells/mL of BEP cells or cortical cells (control) into 2 PVN, and then after 4 weeks they were inoculated with 100,000/0.2 mL/rat of MADB106 cells via jugular vein. FIG. 40A: at sacrifice, 70% to 80% of inoculated animals showed tumor at the position of lung in control but the BEP inoculated animals did not (representative lung figures). Showing multiple tumor foci in lungs of control cell-transplanted rats. FIG. 40B: showing absence of tumor foci in lungs of BEP cell-transplanted rats. FIG. 40C: representative photomicrographs of H&E stained normal and metastatic lung tumor.

FIG. 41 shows the evaluation of effects of BEP-cell transplantation on cancer cell retention and immune functions. Rats were transplanted with 20,000 viable cells/mL of BEP cells or cortical cells (control) into 2 PVN, and then after 4 weeks they were inoculated with 100,000/0.2 mL/rat of MADB106 cells via jugular vein. FIG. 41A: NK cell cytolytic activity and macrophage migration activity in splenocytes or in PBMC at 24 hours after tumor inoculation. FIG. 41B: splenocytes mRNA levels of cytotoxic factors and cytokines genes known to regulate NK cell and macrophage functions at 24 hours after tumor inoculation. FIG. 41C: plasma levels of various cytokines at 24 hours after tumor inoculation. FIG. 41D: NK cell populations in PBMC before and after tumor inoculation, and in splenocytes 24 hours after tumor inoculation. FIG. 41E: macrophage cell populations in PBMC before and after tumor inoculation, and in splenocytes 24 hours after tumor inoculation. n=8-12 rats. *, P<0.05; **, P<0.01; ***, P<0.001 versus control.

FIG. 42 shows autonomic mediation of the beneficial effect of BEP transplants on immune activation and tumor cell metastasis in lung. Animals were treated with the opiate blocker naloxone (NAL; 10 mg/kg body weight), β-receptors agonist MET (0.8 mg/kg body weight), nicotine acetylcholine receptor antagonist MLA (2.5 mg/kg body weight), or vehicle, were inoculated with MADB106 cells for determining immune and tumor clearance responses. FIG. 42A: PBMC-derived NK cells' cytolytic activity. FIG. 42B: PBMC-derived macrophage migration activity. FIG. 42C: percentage of tumor incident as compared with control. n=12 rats. **, P<0.01 versus control of the similarly drug-treated group. FIG. 42D: a hypothetical model showing how autonomic nervous system may mediate BEP neuronal control of immune system.

### DETAILED DESCRIPTION OF THE INVENTION

The present invention is described more fully by way of the following. All references cited are incorporated by reference herein.

Abbreviations: ANOVA, analysis of variance; beta-endorphin, BEP; cAMP, cyclic adenosine monophosphate; dbcAMP, dibutyryl cAMP; DMEM, Dulbecco's modified Eagle's medium; DAPI, 4′-6-Diamidino-2-phenylindole; EDTA, ethylenediaminetetraacetic acid; EGF, epidermal growth factor; FBS, fetal bovine serum; FGF, fibroblast growth factor; GABA, gamma-aminobutyric acid; GFAP, glial fibrillary acidic protein; GnRH, gonadotropin hormone-releasing hormone; HDMEM, HEPES-buffered Dulbecco's Modified Eagle's Medium; IgG, immunoglobulin G; kDa, kilodalton; LIF, limphokine inhibitory factor; MAA, Mem amino acid; MAP2, microtuble-associated protein 2; NF-M, Neurofilament M; NPY, neuropeptide Y; NSC, neuronal stem cell; PACAP, pituitary adenylate cyclase-activating polypeptide; POMC, proopiomelanocortin; PGE1, prostaglandin E1; RT-PCR, reverse transcription-polymerase chain reaction, TH, tyrosine hydroxylase.

Cancer immunotherapy is a growing field that aims at restoring and enhancing immune function to combat oncogenic conditions. NK cells play an important role in preventing cancer growth and metastasis, and expansion of these cells in vivo could be a promising immunotherapeutic strategy against cancer either alone or in combination with conventional therapies. However, the use of autologous natural killer (NK) cells is limited due to the fact that selective NK expansion is difficult to achieve in human patients. Opioid peptides can activate NK cell functions in both laboratory animals and in humans. Whether beta-endorphin (BEP) cell therapy may have a significant impact on activating NK cell function to clear cancers cells has not been tested. There was no method to prepare viable primary BEP neurons from the hypothalamus, so it was not feasible to conduct a primary cell replacement therapy study for determining the biological responses. Hence, the feasibility of differentiating neuronal stem cells (NSCs) into BEP neurons in vitro was determined. To begin to examine the capacity of NSCs to generate BEP neurons, neurons were purified from embryonic hypothalamic tissues and grew neurospheres in culture using a stem cell-maintaining medium. These neurospheres were maintained in culture for a period of 2 weeks in the presence or absence of added factors including basic fibroblast growth factor (bFGF). Upon dissociation, they developed secondary neurospheres and formed aggregates that expressed nestin and vimentin, protein markers of the immature uncommitted phenotype. The neurosphere can be maintained in culture for several months by regularly changing medium and splitting cells. The neurospheres were considered NSCs. Pituitary adenylate cyclase-activating peptide (PACAP), a cyclic adenosine monophosphate (cAMP)-activating agent, is highly expressed in the hypothalamus during the period when many neuroendocrine neurons differentiate from the neuronal stem cells (NSCs). The effect of dibutyryl cAMP (dbcAMP) was tested in combination with the PACAP on the differentiation of rat fetal NSCs in primary cultures. The differentiated cells produced neuroendocrine protein BEP but not gonadotropin hormone-releasing hormone (GnRH), neuropeptide Y (NPY) and tyrosine hydroxylase (TH). These cells expressed the BEP peptide-producing gene proopiomelanocortin, and produced an increased amount of the gene and the peptide in response to a regulatory hormone, prostaglandin E. These results indicate that cAMP-elevating agents are involved in differentiation of NSC to BEP neuron. When these cells were transplanted into the paraventricular nuclei (PVN) of the hypothalamus, they maintained functionality for a prolonged period of time, as they show BEP immunoreactivity, POMC gene production and the ability to reduce corticotropin releasing hormone (CRH) gene expression following administration of lipopolysaccaride (LPS).

It was also investigated whether NSC-BEP cell transplants for a period of 3 weeks activate NK cell function. This was studied by transplanting NSC-BEP cells or control cells in immune-deficient neonatally alcohol-fed male rats during the adult period in one PVN or both PVNs in alcohol-fed and control-fed rats. Cortical cells were used as a control for NSC-BEP. Determination of NK cell functions revealed that NSC-BEP transplants significantly increased NK cell cytolytic activity both in the spleens and in peripheral blood mononuclear cells (PBMC) and IFN-gamma levels in plasma in control-fed and alcohol-fed rats. The activation of NK cytolytic function and plasma levels of IFN-gamma by NSC-BEP cells were higher when transplanted in both PVNs as compared to only one PVN. Bilateral NSC-BEP cell transplants in both PVNs were also able to increase NK cytolytic function and plasma levels of IFN-gamma in alcohol-fed animals. However, NSC-BEP cell transplants decreased TNF-alpha levels in the plasma of alcohol-fed and control-fed animals. The levels of both basal and LPS-induced NK cytolytic function and IFN-gamma mRNA levels in splenic tissues of alcohol-fed animals transplanted with NSC-BEP neurons in the PVN were higher than levels in alcohol-fed animals with sham transplants in the PVN. These results indicate that NSC-BEP cell transplants are effective in activating NK cell functions.

Because NK cells are one of the cellular mediators of innate defense and are crucial for defense against infectious diseases and cancer, it is suspected that NSC-BEP cell transplants may alter tumor cell growth. The effects of the cell transplants on carcinogen (N-nitroso-N-methylurea; NMU) and hormone (testosterone)-induced prostate tumor growth was determined. In this experiment control fed male rats were transplanted with NSC-BEP cells, cortical cells or NSC cells in both PVN and then were treated with carcinogen and hormones. The wet weights of total prostate/kg of body weight were 3-5-fold higher in hormone and carcinogen-treated animals than the untreated control animals. The wet weights of total prostate were 3-4-fold lower in animals treated with NSC-BEP cells than those treated with cortical cells or NSC cells. Limited histopathological data revealed that the prostate of animals treated with control cell transplants have either adenoma or adenocarcinoma but the prostate of animal treated with NSC-BEP cells showed free of adenoma or adenocarcinoma. These data indicate that NSC-BEP cell therapy has the ability to increase the body's innate immunity to prevent cancer growth and progression.

Cancer immunotherapy is a growing field that aims at restoring and enhancing immune function to combat oncogenic conditions. One target of this field is the NK cell. As part of the innate immune system, NK cells form the first line of defense against pathogens or transformed/cancerous host cells. In addition, NK cells are likely to interact with potent antigen-presenting dendritic cells, thus forming a bridge between innate and adaptive immunity. Recent experimental and clinical data show the possibility of exploiting NK activity as a cell-based immunotherapy to treat cancer (Arai et al. (2005) Expert Opin. Biol. Ther., 5:163-172). Results from stem cell transplants containing alloreactive donor NK cells and in vitro work indicate a great antitumor potential of NK cells (Chaudhuri et al. (2005) J. Exp. Clin. Cancer Res., 24:165-173).

The natural killer cell is a critical component of the innate immune system and plays a central role in host defense against tumor and virus-infected cells. The importance of the NK cell in controlling tumor growth and metastasis of breast cancer cells has been clearly demonstrated in severe combined immunodeficiency (SCID) mice. It has been shown that breast cancer cells, following inoculation, were efficient in forming large tumors and spontaneous organ-metastasis in NOD/SCID/gammac (null) (NOG) mice lacking T, B and NK cells. In contrast, breast cancer cells produced a small tumor at the inoculated site and completely failed to metastasize into various organs in T and B cell knockout NOD/SCID mice with NK cells. Immunosuppression of NOD/SCID mice by treatment with an anti-maurine TM-beta1 antibody, which transiently abrogates NK cell activity in vivo, resulted in enhanced tumor formation and organ-metastasis in comparison with non-treated NOD/SCID mice. Activated NK cells inhibited tumor growth in vivo. These results indicate that NK cells play an important role in cancer growth and metastasis and are a promising immuno therapeutic strategy against cancer, either alone, or in combination with conventional therapy (Dewan et al. (2005) Biomed. Pharmacother., 59:S375-379; Dewan et al. (2007) Breast Cancer Res. Treat., 104:267-75).

NK cells are one of the cellular mediators of innate defense. They can recognize and kill aberrant cells and rapidly produce soluble factors (chemokines and cytokines) that have antimicrobial effects or that prime other cells of the immune system (Janeway et al. (2002) Annu. Rev. Immunol., 20:197-216). Heterogeneous arsenal of surface receptors that allow NK cells to respond to microbial products, cytokines, stress signals and inducible molecules are expressed after target-cell transformation (Colucci et al. (2003) Nat. Rev. Immunol., 3:413-425). NK cells are therefore crucial for defense against infectious diseases and cancer. They play a vital role in cellular resistance to malignancy and tumor metastasis (Miller, J. S. (2001) Exp. Hematol., 29:1157-1168; Colucci et al. (2003) Nat. Rev. Immunol., 3:413-425). NK cells can destroy infected and malignant cells by calcium-dependent release of cytolytic granules, by activation of the Fas (CD95)-mediated pathway, or by tumor necrosis factor-alpha (TNF-α) release (Taylor et al. (2000) J. Immunol., 165:5048-5053) and activating TNF-α-related apoptosis-inducing ligand (TRAIL)-dependent receptors (Smith et al. (2002) Nature Rev. Cancer 2:850-861). Among these mechanisms, the release of cytolytic granules containing granzymes (particularly granzyme B) and perforin is the major mechanism used for killing the target cell (Barry et al. (2002) Nat. Rev. Immunol., 2:401-409; Raja et al. (2002) J. Biol. Chem., 277:49523-49530). Perforin creates transmembrane pores in the target cell membrane thereby allowing the entry of granzyme B, which then activates the caspase-driven apoptotic pathway. NK cells differ from other cytotoxic effector cell types (e.g., cytotoxic T lymphocytes) in two major ways. They kill the target cells in a non-major histocompatibility complex (MHC)-restricted fashion without the need for previous in vitro or in vivo activation, and only NK cells constitutively express the lytic machinery (Trinchieri, G. (1989) Adv. Immunol., 47:187-376; Moretta et al. (2002) Eur. J. Immunol., 32:1205-1211). In addition, NK cells can perform antibody-dependent cell-mediated cytotoxicity (ADCC), which involves the lysis of antibody-coated targets (Perussia et al. (1983) J. Immunol., 130:2133-2141). Another essential function of NK cells is the production of cytokines such as interferon-gamma (IFN-γ), (TNF-α), and granular macrophage cell-stimulating factor (Trinchieri, G. (1989) Adv. Immunol., 47:187-376). NK cells are highly efficient in the cellular immune response against malignant tumors without restriction of major histocompatibility complex. However, clinical studies using autologous NK cells have been reported in only a very limited number of cases, due to the fact that selective NK expansion is difficult to achieve in this patient population (Ishikawa et al. (2004) Anticancer Res., 24:1861-1871).

One of the endogenous peptides that control NK cell function is beta-endorphin (BEP). Cells producing this peptide are localized in the hypothalamus. These neurons are shown to be important regulators of NK cell activity (Boyadjieva et al. (2001) J. Immunol., 167:5645-5652; Boyadjieva et al. (2002) Clin. Exp. Res., 26:1719-1727; Boyadjieva et al. (2004) J. Immunol., 173:42-49; Dokur et al. (2004) J. Neuroimmunol., 151:148-157; Dokur et al. (2005) J. Neuroimmunol., 166:29-33; Dokur et al. (2004) Alcohol Clin. Exp. Res., 28:1180-1186). Opioid peptides can affect the NK cell functions by several different mechanisms. It has been shown that endogenous opioids up-regulate human and rat NK cell activity (Faith et al. (1984) Clin. Immunol. Immunopathol., 31:412-418; Kay et al. (1984) Life Sci., 35:53-59; Matthews et al. (1983) J. Immunol., 130:1658-1662). The cytolytic activity of NK cells can be enhanced by lymphokines such as IFN-gamma and interleukin-2 (IL-2) (Ortaldo et al, 1983, Santoli et al, 1987). The effects of IFN-gamma on NK cells can be blocked by the opioid antagonist naloxone (Kay et al. (1984) Life Sci., 35:53-59). In addition, IL-2 and IFN-gamma have been shown to bind to opioid receptors (Ahmed et al, 1984). Comparison of the effects of IFN-gamma and BEP revealed that NK activity is enhanced by these two agents to the same magnitude (Dafny, N. (1983) Life Sci., 32:303-305). In addition, IFN-gamma has been shown to have a number of opioid-like effects (Dafny, N. (1983) Life Sci., 32:303-305). Hence, it appears that BEP may regulate IFN-gamma and IL-2-induced NK cell functions. Whether or not BEP neuronal cell transplants activate NK cell function has not previously been reported.

BEP neurons originate in the arcuate nuclei of the hypothalamus and distributed throughout the central nervous system. These neurons are involved in maintaining a variety of functions including mood, food intake, reproduction and body immune function (Boyadjieva et al. (2001) J. Immunol., 167:5645-5652; Cone et al. (2001) Int. J. Obes. Relat. Metab. Disord., 25:S63-67; Smith et al. (2001) Reprod., 122:1-10; Terenius, L. (2000) Ups. J. Med. Sci., 105:1-15). Lower numbers of BEP-expressing arcuate nucleus neurons have been found in various brain pathologies including schizophrenia and depression (Bernstein et al. (2002) Cell Mol. Biol., 48:259-265; Zangen et al. (2002) Neurosci., 110:389-393). Mutation of proopiomelanocortin (POMC) gene producing BEP has been observed in obese patients (Pankov et al. (2002) Vopr Med Khim, 48:121-130).

NSCs can be isolated, for example, from various parts of the brain, maintained in cultures as neurospheres in the presence of mitogens and expanded and differentiated into neurons, astrocytes and oligodendrocytes in the presence of various neurotrophic factors (Roisen et al. (2001) Brain Res., 890:11-22; Roybon et al. (2004) Cell Tissue Res., 318:261-273). However, the neuronal differentiation properties of NSCs can depend on the region of the brain from which they have been isolated. For example, cortical neurospheres produce more dopaminergic neurons than ventral mesenchephalic neurospheres. This regional specification may make NSCs more suitable for directed differentiation of a specific neuronal phenotype.

The hypothalamus consists of several groups of hormone-secreting neurons that are critical for various neuroendocrine functions (Settle, 2000; Van den Berghe, G. (2000) Euro. J. Endocrinol., 143:1-13). Most of the neurons in the hypothalamus are derived from the proliferative neuroepitelium of the third ventricles and are generated during similar time in embryonic life (Markakis et al. (1997) Brain Res. Rev., 24:255-291). Study of cell development in the rat hypothalamus using ³H-thymidine uptake assays reveal that most of the neurons of the tuberomammillary and arcuate nuclei have late-forming starts, beginning after embryonic day 16 and continuing until birth (Altman et al. (1978) J. Comp. Neurol., 182:995-1015). However, the inductive signal involved in the generation of specific neuronal cell types from these embryonic cells has not been revealed. Using cells from the hypothalamus of 19 days old rat embryos, it has been shown that cyclic adenosine monophosphate (cAMP)-elevating agents have protective action against ethanol-induced death of endorphin neurons (De et al. (1994) J. Biol. Chem., 269:26697-26705). cAMP elevating agents also have been shown to promote β-endorphin neuronal growth and neurite formation (De et al. (1994) J. Biol. Chem., 269:26697-26705; Yang et al. (1993) J. Neuroendocrinol., 5:371-380). These findings have raised the possibility that this neurotrophic factor can be applied to direct heterologous sets of neuronal progenitor cells forming specific neuronal phenotype. In this study, it was determined whether a cAMP analog, dbcAMP, and the cAMP-elevating agent PACAP, could be used to direct the differentiation of hypothalamus-derived NSCs into functional BEP neurons. It was also tested whether in vitro differentiated BEP neurons (NSC-BEP) could be used to activate NK cell function in immune suppressed fetal alcohol-exposed animals. Additionally, it was tested whether the NSC-BEP cell transplants could alter the growth of prostate tumors induced by carcinogen and testosterone.

Prenatal alcohol exposure produces a range of adverse outcomes in offspring, collectively referred to as fetal alcohol spectrum disorders (FASD). The most severely affected children on the spectrum show a series of anomalies including growth retardation, malformations on the face and permanent central nervous system damage, which are called fetal alcohol syndrome (FAS). Fetal alcohol exposure is the leading known cause of mental retardation in the western world. In the United States and Europe, the FAS prevalence rate is estimated to be nearly one in every 100 live births. Apart from mental retardation, fetal alcohol exposed animals often have immune deficiencies, including defects in lymphoid tissue development, immune cell function, humoral immunity and cytokine secretion. Possibly due to these immune deficiencies, they also show a higher rate of infection and higher susceptibility to different types of cancer. Cancer is a class of diseases characterized by uncontrolled abnormal cell growth, invasion, and metastasis. It was shown that chronic high levels of stress can increase carcinogenesis in rat models. Both psychological and physical stresses result in activation of hypothalamus-pituitary-adrenal (HPA) axis and sympathetic nervous system (SNS). Activation of stress axis can be inhibited by β-endorphin (β-EP), which is an endogenous opioid polypeptide known to have the ability to inhibit stress hormone production, produce analgesia and a feeling of well-being. It was observed that activation of the SNS produces detrimental effects on the activity of NK cells, which are important components of innate immune system, and NK cell suppression is correlated with a compromised tumor response against metastasis (Albertsson et al. (2003) Trends Immunol., 24:603-609). On the other hand, reduced β-EP production was observed in patients with a higher incidence of cancers and infections.

Stress is a state of threatened homeostasis by which the body copes with intrinsic or extrinsic adverse forces. The body and mind react to stress by activating a complex and strictly regulated repertoire of central nervous system and peripheral adaptive responses such as activation of the hypothalamus-pituitary-adrenal (HPA) axis. Acute stress responses promote adaptation and survival via responses of neural, cardiovascular, autonomic, immune and metabolic systems. However, excessive or prolonged stress can promote and exacerbate pathophysiology through the same systems that are dysregulated. Chronic stress can significantly affect many of the body's immune systems. For example, higher levels of stress were shown to be associated with increased sympathetic activity, decrease in NK cell lysis activity, decrease in IFN-γ levels, and higher vulnerability to infections such as cold virus. It was reported that chronic high levels of stress can increase carcinogenesis in rat models. Therefore manipulations to control stress response and reduce stress level may turn out to be a promising treatment to increase immunity and fight against diseases. β-endorphin (β-EP), an endogenous opioid polypeptide produced by neurons and immune cells, is known to have the ability to inhibit stress hormone production, produce analgesia and a feeling of well-being. Decrease of β-EP neuronal function was correlated with different types of emotional and physical diseases. For example, lower numbers of β-EP neurons have been reported in brains of patients with schizophrenia, depression, and fetal alcohol syndrome, and reduced β-EP production was observed in obese patients (Bernstein et al. (2002) Cell Mol. Biol., 48: OL259-65; Pankovlu et al. (2002) Vopr Med. Khim, 48:121-30; Kuhn et al. (2008) Alcohol Clin. Exp. Res., 32:706-14). These pathological conditions were found to correlate with higher incidences of cancers and infections (Irwin et al. (2007) Brain Behav. Immun., 21:374-83; Giovannucci et al. (2007) Gastroenterology, 132:2208-25). In-utero alcohol exposed animals was known to have hyperactive HPA axes, defected immune systems and higher incidence of bacterial infections (Johnson et al. (1981) Pediatr Res., 15:908-11). It is found herein that fetal alcohol exposed rats have reduced numbers of β-EP neurons and increased susceptibility to different types of cancer. The correlation between β-EP reduction and immune dysfunction in fetal alcohol exposed animals makes them a good model for investigating relationships between stress and diseases. A method to generate β-EP neurons in vitro from hypothalamic neuronal stem cells (NSCs) has been developed using cyclic adenosine monophosphate (cAMP) in culture. A method has also been developed to induce NSCs to differentiate into β-EP neurons in vivo by injecting cAMP-delivering-nanoparticles (nanosphere) into third ventricle of the brain. It was shown that transplantation of in vivo-differentiated β-EP neurons inhibited growth of chemically induced prostate cancer. Therefore the beneficial effect of β-EP to cure other diseases is shown herein. The preliminary data showed that increased β-EP neurons in hypothalamus significantly reduced growth and progressing of a carcinogen-induced mammary tumors and prevented lung metastasis, possibly by increasing NK cell cytolytic activity and macrophage activity. β-EP neuronal transplants reduce carcinogen-induced tumor growth and prevented lung retention of mammary tumor cells. The effect of fetal ethanol exposure on neuro-immune axis and cancer development is shown herein along with the effect of β-EP cells in recovering immune function in fetal alcohol exposed rats. The mechanism by which β-EP modulates immune function and reduce tumor growth is also determined. These studies reveal the use of in vivo- or in vitro-differentiated β-EP neurons to inhibit growth and metastasis of cancer in both normal and fetal alcohol exposed animals.

Fetal alcohol exposed animals are also shown to have hyperactive HPA axis, higher stress response, and decreased β-EP neuronal function, which may be the cause of immune dysfunction and development of cancer in these animals. Therefore controlling stress level through manipulations to increase β-EP production could be used as a treatment against cancer and diseases caused by immune-deficiency in fetal alcohol exposed.

### Neuroendocrine Response to Stress and its Effect on Immune Function

Stress is a state of threatened or perceived as threatened homeostasis, typically represented by secretion of adrenaline. The stress response is conducted by the stress system, which is located both in the central nervous system and the periphery. Stress is known to cause the release of several stress hormones—particularly glucocorticoids through activation of the hypothalamic-pituitary-adrenal (HPA) axis and catecholamines through the sympathetic nervous system. In response to a stressor, corticotropin-releasing hormone (CRH) and arginine-vasopressin (AVP) are secreted into the hypophyseal portal system and activate neurons of the paraventricular nuclei (PVN) of the hypothalamus. This results in release of adrenocorticotropic hormone (ACTH) from the pituitary into the general bloodstream, which results in secretion of glucocorticoids from the adrenal cortex. In humans, the natural glucocorticoid is cortisol, while in rodents it is corticosterone. The autonomic nervous system (ANS) provides the rapid response to stress commonly known as the fight-or-flight response, engaging the sympathetic nervous system and withdrawing the parasympathetic nervous system, thereby enacting cardiovascular, respiratory, gastrointestinal, renal, and endocrine changes. Activation of the sympathetic nervous system results in secretion of acetylcholine from the pre-ganglionic sympathetic fibers in the adrenal medulla. This induces secretion of epinephrine into the systemic blood supply. Norepinephrine is released from the nerve terminals in the vicinity of immune cells. These catecholamines have many immunomodulatory effects.

Human body and mind response to stress is tightly regulated through the complex system. If reaction to stress is inadequate or excessive and/or prolonged, it may affect many physiological functions such as growth, metabolism, circulation, reproduction, and inflammatory/immune response. Chronic stress can significantly affect the body's immune system. For example, higher levels of stress were shown to be associated with increased sympathetic activity and decrease in NK cell lysis activity and IFN-γ levels. Chronic high levels of stress can increase carcinogenesis in rats. It was reported that intense exercise-induced stress enhances mammary carcinogenesis in the rat model. Stress can also be associated with increased prevalence of tuberculosis and vulnerability to common cold virus. Contrarily, behavioral interventions aimed at reducing stress and increasing optimism in cancer patients have been shown to enhance immunity and to reduce tumor growth in breast and prostate cancer patients. The newly developed term “psychoneuroimmunology” is used to describe the interactions between the mental state, nervous and immune systems, as well as research on the interconnections of these systems.

Hormones produced in reaction to stress have detrimental effect on immune functions, including reduced NK cell activity, lymphocyte population, lymphocyte proliferation, antibody production and reactivation of latent viral infections. It is known that glucocorticoids can inhibit all the components of the immune response. Glucocorticoids modulate the transcription of many cytokines. They suppress the proinflammatory cytokines IL-1, IL-2, IL-6, IL-8, IL-11, IL-12, TNF-α, IFN-γ, and GM-CSF while upregulating the anti-inflammatory cytokines IL-4 and IL-10. Glucocorticoids reduce the trafficking of leukocytes to areas of inflammation, inhibit expression of chemoattractants and cell migration, and suppress production of inflammatory mediators such as prostaglandins and nitric oxide. They also suppress maturation, differentiation and proliferation of immune cells involved in all aspects of immunity, including innate, T cell, and B cell function and chronic allergic reactions. Glucocorticoids induce apoptosis in macrophages and monocytes, and inhibit the activation and function of neutrophils. Norepinephrine (NE) released by SNS activation also disturbs inflammatory cytokine network by inhibiting the production of immune-enhancing cytokines like IL-12 and TNF-α, and by up-regulating the production of inhibitory cytokines like IL-10 and TGF-β.

NK cells are a subset of lymphocytes, providing first line defense against viral infection, tumor growth and metastasis by their unique cytolytic action. Cytolytic activity of NK cells involves the synergistic action of pore-forming protein perforin and the serine protease granzyme B to cause apoptosis of target cells (Graubert et al. (1996) Blood 87:1232-7). In addition, NK cells are likely to interact with the antigen-presenting dendritic cell and T-helper cells, to form a bridge between innate and adaptive immunity. NK cells are able to spontaneously detect major histocompatibility complex (MHC) class I-deficient tumor cells and destroy infected and malignant cells by calcium-dependent release of cytolytic granules, by activation of the Fas (CD95)-mediated pathway, or by tumor necrosis factor-alpha (TNF-α) release and activating TNF-α-related apoptosis-inducing ligand (TRAIL)-dependent receptors. Following activation, NK cells produce IFN-γ, which plays an important role in antiviral defense, probably by enhancing nitric oxide production and modifying a variety of molecules important for replication of particular viruses. NK cells are sensitive to stress, and their function is influenced by psychoneurological factors. Among the HPA hormones, glucocorticoid and CRH have been shown to be potent inhibitors of NK cell activity in vitro and in vivo. Hypothalamic CRH neurons control the functions of spleens via the sympathetic nervous system (Irwin et al. (1990) J. Pharmacol. Exp. Ther., 255:101-7). It has been shown that exogenous CRH suppresses immune function in both normal and adrenalectomized rats, indicating an inhibition mechanism other than activation of chorticosterone. Microinjection of CRH into lateral ventricle of the brain increases noradrenergic function and inhibits NK cell activity in rat spleen, while peripheral administrated anti-CRH serum has no effect on NK cell cytolytic activity. Pretreatment of the animals with sympathectomy significantly reduced splenic norepinerphrine concentration and completely abolished both the CRH-induced increase in plasma catecholamine levels and the reduction in splenic NK activity. β-adrenergic receptors were found on NK cells. Blocking of β-adrenergic receptor antagonized the CRH-induced reduction in NK activity. Circulating levels of ACTH and corticosterone, or activation of pituitary adrenal axis by CRH, was dissociated from changes in NK activity. These data show that CRH inhibits NK activity through activation of SNS, which causes release of catecholamines in spleen and activation of β-adrenergic receptor.

Sympathetic modulation of immune function may involve the direct effect of catecholamines on lymphocytes or may result from circulatory distribution of immune cells between lymphoid organs and peripheral blood. In vitro treatment of human cells with catecholamines suppressed the NK cell activity. NK cell function can be altered by blocking ANS transmission with ganglionic blocker, chlorisondamine, which exerts a persistent nicotinic blockade within the ANS. In vivo administration of adrenergic agents suppressed NK cell activity in rats. This effect was independent of the transitory increase in the number of NK cells (Shakhar, et al. (1998) J. Immunol., 160:3251-8), suggesting that the decline in NK activity is independent of NK cell distribution. In whole splenocyte culture, NE has inhibitory effect on NK cell activity and mRNA levels of IFN-γ. NE and other β-adrenergic agents alter the levels of the cytotoxic molecules, perforin and granzyme B. The inhibitory effect of MP on the granzyme B level was completely blocked by nadolol, a non-selective β-adrenergic blocker. NE-mediated effects are through stimulation of α- and β-adrenergicreceptors present on target cells. All the lymphoid cells, except Th2 cells, are known to express β-adrenergic receptors. NK cells possess the highest number of β-adrenergic receptors when compared to monocytes, CTLs, B cells and helper cells, indicating a higher susceptibility of NK cells to stress-induced SNS activation.

Monocyte/macrophage is another important component of immunity against cancer which can be inhibited by stress hormones. Mononuclear phagocyte system consists of peripheral blood monocytes and tissue macrophages. The monocyte system serves a pivotal role in immune surveillance against micro-organisms and malignant cells and contributes equally and highly effectively to specific and non-specific immunological reactions. Cytokines that are secreted as an integral component of the innate immune response such as granulocyte-macrophage colony-stimulating factor (GM-CSF) and IFN may directly activate the functions of the monocyte system. Activated monocytes exhibit enhanced phagocytic activity for micro-organisms, tumor cells or apoptotic cells as well as an up-regulation in the respiratory burst activity that liberates free oxygen radicals and superoxides that are toxic to foreign cells. Monocytes can be induced to express and secrete inflammatory cytokines such as TNF, IL-1 and IL-6. Monocytes interact with T and B lymphocytes and can therefore modulate the specific and adaptive immune response.

In summary, hormones secreted during stress from HPA axis and SNS have inhibitory effects on immune functions against infection and cancer. Therefore ways to reduce the detrimental effect caused by hyperactive stress axis form a novel method in recovering immunity in a variety of cases.

### β-Endorphin in Stress Regulation

Endorphins are endogenous opioid polypeptide compounds, a cleavage product of pro-opiomelanocortin (POMC) which is also the precursor hormone for adrenocorticotrophic hormone (ACTH). They are produced by the pituitary gland and the hypothalamus in vertebrates during exercise, excitement, pain, consumption of spicy food and orgasm, and they resemble the opiates in their abilities to produce analgesia and a feeling of well-being. β-EP neuronal cell bodies are primarily localized in the arcuate nuclei of the hypothalamus, and its terminals are distributed throughout the central nervous system, including many areas of the hypothalamus and limbic system. β-endorphin is released into blood from the pituitary gland and into the spinal cord and brain from hypothalamic neurons. β-EP neurons innervate CRH neurons in the PVN and inhibits CRH release, while naltrexone, a μ-opioid receptor antagonist, increases it. During stress, secretion of CRH and catecholamines stimulates secretion of hypothalamic β-endorphin (β-EP) and other POMC-derived peptides, which in turn inhibit the activity of the stress system. Hence, it appears that hypothalamic β-EP inhibits CRH secretion, and by doing so, regulates sympathetic outflow to the spleen and positively regulates NK cell cytolytic activity.

β-EP is involved in immune functions. Abnormalities in β-EP neuronal function are correlated with different types of emotional and physical diseases. For example, lower numbers of β-EP neurons have been reported in brains of patients with schizophrenia, depression, and fetal alcohol syndrome (Zangen et al. (2002) Neuroscience, 110:389-93). Reduced β-EP production was observed in obese patients (Pankov et al., Vopr Med Khim, 48:121-30), and a higher incidence of cancers and infections was found to correlate with these pathological conditions (Giovannucci et al. (2007) Gastroenterology, 132:2208-25). β-EP administration into the PVN stimulates sympathetic outflow and catecholamine levels in circulation, and inhibit norepinephrine secretion in various parts of the brain. When injected into different areas of the brain, β-EP affects sympathetic outflow differently, which suggests that β-EP affects sympathetic outflow through nervous system rather than circulatory system.

β-EP was also found in the immune system such as T-helper cells and spleen macrophages. In vitro exposure of splenic lymphocytes to β-EP was shown to increase NK cell activity, and this effect was blocked by opiate antagonist naltrexone (Dokur et al. (2005) J. Neuroimmunol., 166:29-38). Physiologically, elevated β-EP levels in circulation were shown to be associated with increased NK cell activity. Persistently low levels of β-EP in circulation are suggested as a risk factor for infectous diseases and depression. It has been shown that NK cell function can be altered by chlorisondamine, a persistent nicotinic blocker of SNS. β-EP's stimulatory effect on NK cells is also blocked by chlorisondamine, suggesting that the opioid stimulatory signal to the spleen is also transmitted through ANS.

In the CNS, β-EP is known to bind to classical δ- and μ-opioid receptors and modulate the neurotransmission in sympathetic neurons to alter NK cell cytolytic functions in the spleen. β-endorphin has the highest affinity for the μl opioid receptor, slightly lower affinity for the μ2 and δ opioid receptors and low affinity for the κ1 opioid receptors (Brownstein, M. J. (1993) Proc Natl Acad Sci, 90:5391-3). β-opioid receptor-specific agonists have been shown to stimulate NK cell cytolytic activity, and intracranial administration of δ-specific antagonist blocks intracranial β-EP-stimulated NK cell function (Dokur et al. (2005) J. Neuroimmunol., 166:29-38). δ-like opioid receptors were reported to be involved in regulation of immune cells. The selective δ-opioid agonist deltrophin enhanced the Con A-induced proliferation of murine splenocytes (Boyadjieva et al. (2002) Alcohol Clin Exp Res, 26:1719-27). Perfusion of β-EP in the PVN increases lymphocyte responses to Con A, PHA, and LPS in proliferation assay and this effect is partially inhibited by the selective δ-opiate receptor antagonist naltrindol (Dokur et al. (2004) Alcohol Clin Exp Res, 28:1180-6). Therefore it is possible that β-EP modulate the proliferation of immune cells partly by acting as an agonist on δ-opioid receptors.

Opioid receptors are present on lymphocytes. NK cell cytolytic activity is increased by IFN-γ, a lymphokine that has a number of opioid-like effects. Both IFN-γ and IL-2 are shown to hind opioid receptors, and enhance NK cell cytolytic activity. IFN-γ and IL-2-mediated NK cell cytolytic activity is blocked by the opioid receptor antagonist naloxone. Therefore it appears that β-EP may regulate IFN-γ and IL-2-induced NK cell functions. In vivo and in vitro studies also showed that both central and peripheral administration of β-EP increase NK cell activity and that an opioid receptor antagonist, naltrexone, blocks these responses. β-EP suppresses IFN-γ production in cultured human PBMCs. PVN infusion of β-EP enhances production of IFN-γ and granzyme B (Dokur et al. (2004) Alcohol Clin Exp Res, 28:1180-6). In vitro study showed that NK cell activity in cultured human PBMCs increases after β-EP, IL-2, or IFN-γ (Boyadjieva et al. (2002) Alcohol Clin Exp Res, 26:1719-27).

Naltrexone, an opioid antagonist, has been used in clinical trials to treat alcoholism. As opioid peptides β-EP increase splenic NK cell function in rats, it is anticipated that naltrexone treatment will cause immunosuppression. However, chronic naltrexone administration in rats increases the cytolytic activity of NK cells. Treatment of naltrexone in mice showed no effect on basal levels of plasma corticosterone. Hence it is unlikely that naltrexone increased cytokines production from spleen via suppressing corticosterone levels. Naltrexone is an opioid antagonist when administered acutely, but shows δ-opioid-like activity following chronic administration (Boyadjieva et al. (2004) J. Immunol., 173:42-9). δ-opioid receptor agonists have been shown to stimulate NK cell cytolytic activity, and a δ-specific antagonist blocks β-EP-stimulated NK cell function. It has been previously shown that administration of naltrexone for a period of 2 weeks suppresses μ-opiate receptor binding but increases δ-opiate receptor activity in rat splenocytes; thus enhances NK cell cytolytic activity response to β-EP in vitro (Boyadjieva et al. (2004) J. Immunol., 173:42-9). Naltrexone chronic administration increased the production of IL-2, IL-4, and IL-6 and the basal and cytokine-activated NK cell activity and IFN-γ production. Naltrexone treatment also increases NK cell cytolytic activity and cytokine production in the spleen in vivo (Boyadjieva et al. (2004) J. Immunol., 173:42-9).

The δ-opioid receptor agonistic like activity of naltrexone on splenocytes appears to be due to its potent μ-opioid receptor antagonistic function. δ-opioid receptor expression in the splenocytes is tightly controlled by a negative feedback regulation of μ-opioid receptors. Naltrexone disrupts this feedback control by reducing μ-opioid receptor function, thereby up-regulating δ-opioid receptor binding which results in enhanced NK cell cytolytic response to the ligands. Naltrexone preferentially prevented δ-opioid receptor activity but increased δ-opioid receptor activity following chronic administration (Boyadjieva et al. (2004) J. Immunol., 173:42-9). Naltrexone treatment enhanced NK cell responses to endorphin, enkephalin, and the δ-opioid agonist, while inhibited NK cell response to μ-opioid receptor agonist (Boyadjieva et al. (2004) J. Immunol., 173:42-9). Naltrexone increased δ ligand binding and decreased μ ligand binding in splenocytes. In contrast, treatment with the δ-specific antagonist naltrindole increased μ ligand binding and decreased δ ligand binding (Boyadjieva et al. (2004) J. Immunol., 173:42-9). Opioid receptors are members of the large superfamily of G protein-coupled receptors. Like many G protein-coupled receptors, δ and μ opioid receptors are constitutively active, producing spontaneous regulation of G protein and effectors in the absence of agonists. Opioid receptors form dimers in order to function. There is a hypothesis that δ and μ may form heterodimers in tissue, by decreasing the μ opioid receptors, naltrexone un-coupled the μ and δ dimmers, thereby leading to increased δ ligand binding sites and increased endogenous opioid-activated NK cell function in splenotytes. An alternative explanation is that in the spleen, constitutively active μ receptors inhibited the ligand binding ability of the δ receptors, and therefore by down-regulating μ receptor expression, naltrexone increased δ receptor function (Boyadjieva et al. (2004) J. Immunol., 173:42-9).

With the effects on stimulating δ-opioid receptors and NK cell activity, it is shown here that chronic naltrexone may be used to potentiate the immune-enhancing function of β-EP neurons.

### Fetal Alcohol Syndrome, β-EP, and Epigenetic Modification

Fetal alcohol spectrum disorders (FASDs) is a disorder that occurs to the embryo when a pregnant woman ingests alcohol during pregnancy, causing a wide range of deficits in growth, anatomy, cognition and behavior. Fetal alcohol syndrome (FAS), the most severe of the FASDs, includes prenatal and/or postnatal growth retardation, CNS dysfunction, and a defining pattern of craniofacial malformations. Embryonic exposure to ethanol reduces the number of neurons in various parts of the central nervous system including the hypothalamus. Alcohol abuse is also known to result in clinical abnormalities of endocrine function and neuroendocrine regulation, possibly through two pathways: one is that alcohol crosses the placenta and directly affects developing fetal cells and tissues; another is that alcohol-induced changes in maternal endocrine function disrupts hormonal interactions between mother and fetus. Maternal alcohol consumption increases HPA activity in both maternal female and the offspring, and increased exposure to endogenous glucocorticoids throughout the lifespan can alter behavioral and physiologic responsiveness and increase vulnerability to illnesses or disorders later in life. Ethanol-exposed fetuses exhibit significantly lower corticosterone levels than control fetuses on Day 19 of gestation. However, at birth ethanol-exposed neonates have elevated plasma and brain levels of corticosterone and elevated plasma but reduced pituitary levels of β-EP. It is known that maternal alcohol consumption increases maternal adrenal weight, basal corticosterone levels, and the corticosterone response to stress, while maternal adrenalectomy reversed the effects of prenatal ethanol on pituitary pro-opiomelanocortin (POMC) mRNA levels in ethanol-exposed offspring. These data indicate that fetal stress axis programming is regulated by maternal corticosterone elevation cause by alcohol exposure.

Another system that appears to be susceptible to ethanol during the fetal life is the immune system. Alterations in immune function may be one of the long-term consequences of fetal HPA programming. Children prenatally exposed to alcohol have lower cell counts of eosinophils and neutrophils, decreased circulating E-rosette-forming lymphocytes, reduced mitogen-stimulated proliferative responses by peripheral blood leukocytes, and hypo-c-globulinemia. Prenatal ethanol exposure in animal models is shown to negatively affect lymphoid tissue development, immune cell function, humoral immunity and cytokine secretion. Activity of NK cells, the first line of immune defense against infection and cancer, is suppressed in FAS. As a consequence, children prenatally exposed to alcohol often have an increased incidence of bacterial infections such as urinary tract and upper respiratory tract infections.

What is more, these abnormalities continue to the adulthood, possibly due to the programming of tissue that exhibit persistent alterations in morphology and gene expression. For example, both fetal alcohol exposed adult males and females exhibit increased corticosterone and ACTH responses to stressors such as repeated restraint, footshock, and immune challenges. Both males and females also show increased immediate early gene and CRH mRNA levels following stress. Increased stress axis activity and diminished immune system throughout the lifespan can cause alteration in physiology and susceptibility to different kinds of disorders and diseases including cancer. Recent studies showed that fetal alcohol exposed animals have higher incidence of breast and prostate neoplasia under carcinogen induction. Increased HPA activity could result from increased secretion of secretagogues, or deficits in feedback regulation of HPA activity. β-EP is an inhibitor of CRH and down-regulates the activity of stress axis. It has recently been found that prenatal exposure to ethanol causes a significant decrease in the number of β-EP neurons in the hypothalamus in rats by suppressing the proliferation of neuronal precursors and/or enhancing cell death. In vitro study showed that moderate dose of ethanol inhibited the differentiation and maturation of β-EP neurons by cAMP-activating agents in primary cultures of fetal hypothalamic neuronal progenitor cells (De et al. (1999) Alcohol Clin Exp Res, 23:46-51). It is already known that ethanol induces β-EP neuronal apoptotic death (Sarkar et al., (2007) Endocrinology, 148:2828-34). By using an early postnatal binge ethanol administration model, it was shown that ethanol also reduces the number of β-EP neurons differentiated from neuronal progenitor cells in neonatal rats. It was shown that chronic (more than 2 weeks) alcohol cause immune deficiency such as decrease of NK cell activity and cytokine production, as well as decrease of β-EP peptide production in rat models. This deficiency can be reversed by intracranial administration of β-EP (Boyadjieva et al. (2001) J. Immunol., 167:5645-52).

A growing body of scientific literatures suggests that environmental factors acting prenatally on the developing fetus determine disorders later in life by modulating epigenetic imprint of important tissue. It has been shown that in fetal alcohol exposed animals, the reduced β-EP function is associated with low expression of proopiomelanocortin (POMC) mRNA, increased DNA methylation of the POMC gene and increased production of histone and DNA methyltransferases in β-EP neurons in the hypothalamus. Therefore decreased β-EP neuron function by hypermethylation may be one factor that causes hyperactivity of stress axis and deficiency in immune system in fetal alcohol exposed animals.

It has been shown that adult fetal alcohol exposed offspring had epigenetic marks and increased methylation of POMC DNA and a reduction in POMC gene expression in the hypothalamus. It has also been observed that the fetal alcohol exposure increased basal plasma level of corticosterone in both male and female rats during adulthood. Additionally, the LPS-induced corticosterone level was much higher in alcohol-fed than in control-fed rats. The increased basal and LPS-induced corticosterone levels in male offspring of alcohol-fed males bred with control females persisted for both F2 and F3 generations. However, both male and female offspring of alcohol-fed females bred with normal males showed normal basal and LPS-induced corticosterone levels in F2 and F3 generations. Since the offspring (F1) and its germ cells (F2) were exposed to alcohol during development via F0 female, these results suggest for a trans-generation effects in fetal alcohol exposed offspring transmitted via male germ line. It has been shown that fetal-alcohol-altered POMC gene methylation and expression and the stress axis abnormality maintained for several generations, and these effects were transmitted via male germ line. These results identify an epigenetic molecular mechanism potentially underlying lifelong and transgenerational perpetuation of stress hyperactivity incited by fetal ethanol. However, how is this modification passed down to the next generations, and why is it restricted in male germ line is now known. Herein, the mechanism through which fetal alcohol effect can be passed down through generations is shown. Moreover, whether these abnormalities in HPA axis co-exist with defects in immune system in these offspring the same way as in first generation fetal alcohol exposed animals is studied.

With the fact that β-EP production is decreased in fetal alcohol animals and their offspring, replenishing β-EP into the hypothalamus, the effect of fetal alcohol exposure was reversed, the hyper-activity of stress axis was decreased, and the immune function was recovered.

### β-EP Neuron Differentiation and Transplantation

The hyper-active stress axis and immune deficiency in fetal alcohol exposed animals are primarily caused by decreased production of β-EP peptide in central nervous system. Therefore, one can inhibit the hyper-activity in stress axis and recover the immune function in fetal alcohol animals by increase β-EP content in the hypothalamus. Here two different methods are used: to transplant in vitro differentiated β-EP neurons into hypothalamus, or to inject molecules which induce in vivo differentiation of neuronal stem cells into β-EP cells.

Recent studies using neural precursor cells showed a great potential for effective and safe replacement therapy into both the intact and injured central nervous system (CNS). Neuronal stem cells (NSCs) can be isolated, for example, from various parts of the brain and differentiated into neurons, astrocytes and oligodendrocytes in the presence of various neurotrophic factors. The neuronal differentiation properties of NSCs show dependency upon the brain regions from which they have been isolated. A method to differentiate β-EP neuronal precursor cells from NSCs was developed. β-EP neurons are a group of hormone-secreting neurons that primarily exists in the arcuate nuclei of hypothalamus. Most of the neurons in the hypothalamus are derived from the proliferative neuroepithelium of the third ventricle (van Eerdenburg et al. (1994) Brain Res Dev., 79:290-6). It has been shown that in vitro cultured rat hypothalamic neuronal stem cell can be differentiated into β-EP neurons by using an analog of cyclic adenosine monophosphate (cAMP), dbcAMP, and PACAP (pituitary adenylate cyclase-activating peptide) as inductive signals. These neurons showed immunostaining for β-EP and expressed the gene POMC, as they do in vivo. When transplanted in to PVN in the hypothalamus, these in vitro-differentiated β-EP neurons can be integrated into this site, and produce β-EP peptide hormone. After transplantation, the neurons develop many β-EP-immunoreactive neurofibers, produce POMC mRNA, and reduce the response of corticotrophin-releasing hormone (CRH) to a lipopolysaccharide (LPS) challenge in rats. Increased β-EP peptide level can be detected in hypothalamus, but not in blood plasma. These in vitro-produced β-EP neuronal transplants significantly increase NK cell cytolytic activity in the rat spleen and peripheral blood mononeuclear cells (PBMCs), increased IFN-γ and reduce TNF-α levels in plasma. It has been shown that these β-EP transplants can inhibit prostate tumor development in carcinogen-induced rat prostate cancer model, possibly by increasing NK cell activity, reducing the body's inflammatory milieu, and some other mechanisms.

As previously described, it was found that fetal alcohol exposed animals have deficiency in immune system, which may possibly be caused of decreased β-EP production. These transplanted neurons can integrate into surrounding tissue and produce β-EP peptide on site. It has been shown that immune function can be increased by transplantation of these β-EP neurons, possibly through regulation of HPA axis and NK cells activity. In the case of fetal alcohol exposed animals, the immune function is diminished partly by hyperactivity of HPA axis and decrease of β-EP neuronal function. It has recently been shown that in vitro-produced β-EP neuronal transplants can eliminate NK cell functional deficiency and recover the decrease of IFN-γ in fetal alcohol exposed rats. However, it is not known yet whether these transplants can completely eliminate the higher incidence of breast and prostate cancers in fetal alcohol exposed rats. Therefore in this study, it is determined whether β-EP transplants can ameliorate the hyper-activity of HPA axis, decreased immune function, and increased susceptibility to cancers in fetal alcohol exposed animals. Furthermore, whether in vivo nanosphere-induced β-EP neurons can also fight against immune deficiency in fetal alcohol exposed rats examined. It has been shown that β-EP transplanted into PVN of immune-deficient fetal alcohol exposed rats improved NK cell cytolytic function in these rats. The NSC-derived β-EP-producing cells' function is further characterized in reducing breast tumor growth and metastasis in both normal and immune-deficient fetal alcohol exposed rats.

Notably, the study in rats indicates that NSC-derived β-EP cells are not rejected in host animals in various strains. This may be related to the lack of MHC-I protein in NSC. In certain embodiments, β-EP neurons are differentiated from NSCs of the host itself. Since neuronal differentiation from NSC in the neuroepithelium of the third ventricle persists in the adult, which is part of the neuronal plasticity, it is possible to induce NSC to differentiate into β-EP neurons in vivo by administering cAMP-activating agents into the third ventricle, where NSCs are located.

To immobilize and ensure a slow release of cAMP molecules, nanospheres may be used as a carrier. Nanospheres are commercially made spherical particles, typically 13-20 nanometers (nm) in diameter. Each nanosphere can be functionalized with a defined number of certain molecules. Recently, it has been proven that β-EP neurons can be differentiated in situ from neural stem cells by nanosphere-delivered-cAMP into the third ventricle. In this study, it was tested whether the nanosphere-delivered-cAMP into the third ventricle of female rats would decrease the incidence and growth of N-nitroso-N-methylurea (NMU)-induced mammary tumors. The nanosphere delivered-cAMP was able to suppress, possibly via increasing B-EP neuronal cell function, mammary tumor growth.

According to the preliminary data, rats transplanted with cAMP-delivering-nanospheres had significantly reduced level of corticosterone, while naloxone reversed the effect. What is more, cAMP nanosphere transplanted rats had significantly reduced incidence of tumor or inflammation in the lung, while naloxone-α compound that binds to opiate receptors and blocks the effects of morphine and endogenous opioid peptides-injected rats had incidence of tumor similar to control rats. The effect of cAMP-delivering-nanospheres on neuro-endocrine and immune function is further characterized, as well as its role against carcinogen-induced tumors and carcinoma cell line metastasis.

The instant invention identifies the importance of stress maintenance in regulating immune function in cancer patients. Since hypothalamic β-EP is a suppressor of the sympathetic neuronal influence on NK cells and cytokines, β-EP cell therapy has a significant impact on activating immune function in cancer patients. Furthermore, since cAMP-delivering nanospheres induce β-EP neuronal generation in the hypothalamus, nanosphere implantation is a treatment of cancer by increasing immunity against malignancy and metastasis. The data show that β-EP transplantation increases immune function in both normal and fetal alcohol exposed animals, reduces breast cancer development, and eliminates lung metastasis in rats. Similarly, transplantation of nanospheres containing cAMP inhibited HPA axis activity, decreases breast cancer development and eliminates lung metastasis in normal rats. The NSC-B-EP and nanosphere transplant procedure that has been developed, is a major breakthrough in activating immunity to prevent tumor growth and metastasis in both normal and fetal-alcohol exposed cancer patients.

As stated hereinabove, the instant invention encompasses compositions and method for treating, inhibiting (reducing), and/or preventing (substantially or completely reducing probability of developing) cancer, immunological disease, pathogenic infections, inflammation, stress, anxiety and anxiety/stress-related disorders, or fetal alcohol syndrome in a subject in need thereof. In certain embodiments, the methods comprise administering at least one agent which increases β-endorphin, particular β-endorphin neurons. In certain embodiments, the method comprises administering β-endorphin neurons to the subject.

β-endorphin neurons may be induced in vivo or in vitro (e.g., for later administration to a subject (ex vivo)). β-endorphin neurons may be induced by contacting cells, particularly neuronal stem cells, with at least one cyclic adenosine monophosphate (cAMP) or cAMP analog or inducing agent (e.g., neuropeptide PACAP). cAMP analogs include, without limitation, dibutyryl cAMP (dbcAMP), 8-(4-chlorophenylthio)-cAMP, 8-bromo cAMP, and N6 benzoyl cAMP. In a particular embodiment, PACAP and/or dbcAMP is used. Neuronal stem cells may be obtained by any method known in the art, including differentiation of pluripotent stem cells or embryonic stem cells. In a particular embodiment, the neuronal stem cells are obtained from the patient to be treated (autologous).

The agents (compounds or cells) administered to the subject may be administered in a pharmaceutically acceptable carrier (e.g., diluents, preservatives, solubilizers, emulsifiers, adjuvants and/or carriers). Except insofar as any conventional carrier is incompatible with the agent to be administered, its use in the pharmaceutical preparation is contemplated. Compositions of the instant invention may be administered by any method. For example, the compositions of the instant invention can be administered, without limitation, intravenously, intraperitoneally, intrathecally, or intracerbrally. In a particular embodiment, the compositions are administered by direct injection. In a particular embodiment, the agent is administered into the brain, particularly the hypothalamus, more particularly the third ventral.

In certain embodiments, the methods comprise the slow release of agents within the subject. In a particular embodiment, the compounds administered to the subject may be in a nanosphere (also known as a nanoparticle).

In certain embodiments, the instant invention encompasses methods of treating, inhibiting, and/or preventing cancer in a subject in need thereof, by increasing β-endorphin. In a particular embodiment, the cancer is selected from the group consisting of prostate cancer, lung cancer, liver cancer, and breast cancer; particularly prostate cancer or breast cancer. In a particular embodiment, the method comprises treating, inhibiting, and/or preventing metastases. The methods of the instant invention may further comprise the administration of any other conventional anti-cancer therapy. The additional chemotherapy may be administered before, during, and/or after performance of the instant methods. In a particular embodiment, the methods further comprise administering at least one other chemotherapeutic agent. In a particular embodiment, the method further comprises the administration of radiation therapy.

In certain embodiments, the instant invention encompasses methods of treating, inhibiting, and/or preventing an immunological disease, inflammatory disease, pathogenic infection, stress, anxiety, anxiety/stress-related disorders, or fetal alcohol syndrome in a subject in need thereof β-endorphin. In a particular embodiment, the immunological disease or inflammatory disease is selected from the group consisting of rheumatoid arthritis, diabetes (e.g., type II; glucose control), thyroid disorder, celiac disease, obesity, inflammatory bowel syndrome, and lupus. The methods may further comprise the co-administration (e.g., before, during, and/or after) of any other therapy for the disorder to be treated.

The following examples are provided to illustrate certain embodiments of the invention. They are not intended to limit the invention in any way.

### Example 1

BEP neuronal cell bodies are primarily localized in the arcuate nuclei of the hypothalamus, and its terminals are distributed throughout the central nervous system. These neurons are involved in maintaining a variety of functions including stress regulation and immune functions (Plotsky et al. (1993) Ciba Found Symp., 172:59-75; Boyadjieva et al. (2006) Alcohol Clin. Exp. Res., 30:1761-1767). Abnormalities in BEP neuronal function are correlated with various pathologies. For example, lower numbers of BEP neurons have been found in the postmortem brains of patients with schizophrenia and depression (Bernstein et al. (2002) Cell Mol. Biol., 48:OL259-OL265; Zangen et al. (2002) Neuroscience 110:389-393), and a reduced BEP production due to proopiomelanocortin (POMC) gene mutation has been observed in many obese patients (Pankov et al. (2002) Vopr. Med. Khim., 48:121-130). It is noteworthy that a higher incidence of cancers and infection has been found under these pathological conditions (Irwin et al. (2007) Brain Behav. Immun., 21:374-383; Giovannucci et al. (2007) Gastroenterology 132:2208-2225; Grinshpoon et al. (2005) Schizophr. Res., 73:333-341). Furthermore, the endogenous function of BEP neurons is reported to be reduced in cancer patients (Lissoni et al. (1983) Br. J. Cancer 56:834-837). Therefore, it was hypothesized that increased BEP neuronal activity might be beneficial to control the growth of tumors. In this study, it was examined whether a cAMP analog, dbcAMP, and pituitary adenylate cyclase-activating peptide (PACAP) can be used to direct the differentiation of hypothalamus-derived NSCs into functional BEP neurons in vitro, and the functionality of differentiated cells in vivo was determined by transplanting them into the hypothalamus of male rats and evaluating their effects on carcinogen-induced prostate tumors and immune functions.

### Materials and Methods

### Hypothalamic Neuronal Cell Cultures

Fetal brains were obtained from 17-day-old pregnant rats (Simonsen laboratories; Gilroy, Mass.). Mediobasal hypothalamic tissues from these rats were dissected out and cells from these tissues were dissociated, and mixed hypothalamic cell cultures were prepared as previously described (De et al. (1994) J. Biol. Chem., 269:26697-26705). Neurons were separated from glial cells by filtering mixed hypothalamic cells through a 48-μM nylon mesh. Then hypothalamic cells were sedimented at 400 g for 10 minutes; pellets were re-suspended in HEPES-buffered Dulbecco's Modified Eagle's Medium (HDMEM, 4.5 g/l glucose; Sigma, St. Louis, Mo.); and cells were cultured into poly-ornithine coated 25-cm² tissue culture flasks (2.5 million cells/flask) in HDMEM-containing 10% fetal bovine serum (FBS) and 1% penicillin/streptomycin. On day 2, the culture medium was replaced with HDMEM containing 10% FBS, 33.6 μg/ml uridine and 13.6 μg/ml 5-fluodeoxyuridine to prevent the overgrowth of astroglial cells. All of these chemicals were obtained from Sigma. On day 3, the culture medium was replaced with HDMEM-containing serum supplement (30 nM selenium, 20 nM progesterone, 1 μM iron-free human transferrin, 5 μM insulin, and 100 μM putrescin) and 1% penicillin/streptomycin. Cells were maintained for the next 2 days with this medium. By this time, these cultures were approximately 85-90% neurons, as determined by MAP-2 positivity.

### Development of Nestin Positive Cells and Spheres

Enriched hypothalamic neurons were maintained in HDMEM, containing 10% FBS, for 3 weeks. Each week, cells were trypsinized and cultured. By the beginning of the third week, many spheres started to develop. These spheres were separated and dissociated into single cells by using trypsin/EDTA (Sigma) solution. They were then cultured in suspension or in poly-L-ornithine-coated 24-well plates (20,000 cells/well) in stem cell medium (DME F-12, lymphokine inhibitory factor (LIF), 0.1 μg/ml; L-glutamine, 10 mM; rat bFGF, 20 ng/ml; Mem amino acids solution, MAA, 0.5%; all of the chemicals were from Sigma except that bFGF was obtained from R&D Systems, Minneapolis, Minn.). Cells were cultured for 2 weeks, during which they grew and developed secondary spheres. Some of these cultures were used for immunocytochemical localization of nestin staining to determine whether they were neurospheres. These neurospheres were maintained in cultures for several months by regularly changing the medium and by splitting cells. The secondary spheres were re-suspended and cultured in poly-L-ornithine-coated 24-well plates (20,000/well; for physiological studies) or in poly-L-ornithine-coated 8-well permanox slides (1000 cells/slide; NaIg Nunc International Corp., IL; for histochemical studies). The differentiation experiments were performed by treating these cells for 1 week with PACAP (1-10 μM; SynPep) and dbcAMP (1-10 μM; Sigma) or a combination of both, and then in defined cell culture medium without the drugs for 1 week. At day 3, day 7 and day 14 the immunocytochemical, biochemical and/or real-time RT-PCR analyses were performed.

### Animal Surgery/Transplantation

Pregnant Sprague-Dawley rats were purchased and individually housed in 12-hour light/12-hour dark cycles (lights on at 7:00 am) and constant temperature (22° C.) throughout the study. On gestational days 11 to 21, pregnant rats were fed chow ad libitum (ad lib-fed), fed a liquid diet (BioServe Inc., Frenchtown, N.J.) containing ethanol at a level of 36% (ethanol-fed), or pair-fed an isocaloric liquid control diet (with the ethanol calories replaced by maltose-dextrin). Pups were kept with the fostered dams until postnatal day 22 and then weaned, housed by sex, and provided rodent chow meal and water ad libitum.

Differentiated cells were dissociated using 0.05% trypsin/EDTA (Invitrogen), washed and resuspended at a concentration of 20,000 cells/μl in HDME medium containing serum supplement for transplantation. Cells were placed on ice throughout the grafting session. After completion of the grafting session, cell viability was assessed using the Trypan Blue assay. Viability was greater than 90%. The composition of the differentiated cultures, with respect to the absence of undifferentiated NSCs and the presence of mature BEP-producing cells, was verified before grafting by staining for the immature neural marker nestin, and for markers of mature neurons (NeuN and MAP-2), astrocytes (GFAP), and oligodendrocytes (RIP) as well as for BEP.

Animals at forty days of age were anesthetized with sodium pentobarbital (50-70 mg/kg, i.p.; Henry Schein, Indianapolis, Ind.), and injected with 1.0 μl of stem cell suspension into each of two PVN lobes; the coordinates were set 0.5 mm from the midline, 1.8 mm behind bregma, 0.5 mm lateral of bregma, and 7.5 mm below the cortex using a 5 μl Hamilton syringe. Each injection was over a 5-minute duration. Following the injection, the cannula was left in place for 20 minutes as to allow the absorption of the treatment so that it would not be sucked out upon removal of the cannula. The cannula was then slowly removed in small intervals over a 10-minute period. The dura was closed with 9-0 suture, muscle was re-apposed and the skin was closed with wound clips. Animals received Bupranorphin (Reckitt Benckiser; Richmond, Va.) postoperatively. Rats were injected with 30,000 IU of penicillin (Henry Schein, Indianapolis, Ind.) and placed on a heating pad for recovery. Animal surgery and care were performed in accordance with institutional guidelines and complied with NIH policy. No immune suppression was used. The animal protocol was approved by the Rutgers Animal Care and Facilities Committee.

### NSC-BEP Cell Functionality In Vivo

The functionality of the NSC-BEP cells was studied in vivo by determining the changes in the expression of POMC and CRH mRNA in the PVN following systemic administration of LPS between the animals with NSCs transplants and sham-transplants. The LPS-induced changes were also determined in the expression of POMC and CRH mRNA in the PVN in control animals that were untreated with alcohol during fetal life. The 100 μg/kg dose of LPS was used for a period of 3 hours (which was found to be an effective dose; Chen et al. (2006) Alcohol Clin. Exp. Res., 30:1925-1932) to determine the changes in the hypothalamic CRH and BEP responses of the NSCs transplants or control transplants (cortical cell transplants). From the brains of these animals, PVN was collected by punching. The tissues were used to determine POMC mRNA and CRH mRNA levels by real-time RT-PCR methods or BEP and CRH levels by radioimmunoassay.

### Immunohistochemistry

Cell cultures were fixed in 4% paraformaldehyde for 30 min and then in 70% ethanol for an additional 30 minutes. The immunocytochemistry was performed by using a Quick Kit (Vector Laboratories Inc., Burlingame, Calif.) and following the instructions provided in the kit. Cells were incubated with primary antibodies overnight at 4° C. Primary antibodies used were monoclonal antibodies for nestin (BD Pharmingen; San Jose, Calif. 1 μg/ml), vimentin (clone V9, mouse ascites fluid, 0.22 μg/ml; Sigma; 1:40), α-internexin (Santa Cruz Biotechnology, Santa Cruz, Calif.; 1 μg/ml), MAP2 (2A+2B, clone AP-20, mouse ascites fluid, 0.72 μg/ml; Sigma), β-tubulin (type III, clone SDL.3D10, mouse ascites fluid, 0.30 μg/ml, Sigma), GFAP (clone G-A-5, 45 μg/ml, Sigma), NF-M (145 kDa, 5 μg/ml, Chemicon International, Temecula, Calif.), TH (IgG1, 1:500; BD Biosciences), polyclonal primary rabbit antibody for β-endorphin (1:1000; Peninsula Laboratories, San Carlos, Calif.), GnRH (1:500; Chemicon) and NPY (1:500; Peninsula Laboratories). The secondary antibody used to react with mouse primary antibodies (Nestin, MAP2, type III β-tubulin, NF-L, NF-M and alpha-internexin) was Alexa Fluor 488 donkey anti-mouse IgG, (4 μg/ml; Molecular Probes, Eugene, Oreg.) and with rabbit primary antibody (β-endorphin) was the Alexa Fluor 594 donkey anti-rabbit IgG (H+L) (4 μg/ml; Molecular Probes). Both of these secondary antibodies failed to stain the NSC or differentiated cells in the absence of a primary antibody. Some of the cell-containing chambers were dried and mounted using DAPI-containing Mounting Medium (H-1200; Vector Laboratories Inc.). Fluorescent images were captured with a Cool SNAP-pro CCD camera coupled to a Nikon-TE 2000 inverted microscope. Images were processed with Adobe Photoshop 7.0.

### Radioimmunoassay

The immunoreactive β-endorphin levels in culture medium samples were measured by a radioimmunoassay system (De et al. (1994) J. Biol. Chem., 269:26697-26705). All the samples were dried and reconstituted in assay buffer and measured in duplicate in a single assay. The minimum amount of β-endorphin detectable was 3 pg/tube.

### Real-Time RT-PCR Analysis

Expression levels of POMC mRNA in NSCs and differentiated cells were measured by a quantitative real-time RT-PCR (TaqMan assay) on an ABI PRISM 7700 Sequence Detector (PerkinElmer Applied Biosystems, Foster City, Calif.) as described previously (Chen et al. (2004) J. Neurochem., 88:1547-1554). This assay is based on the 5 nuclease activity of Taq DNA polymerase for fragmentation of a dual-labeled fluorogenic hybridization probe and was performed following the manufacturer's instructions. Total RNA was isolated from the differentiated cultures using an RNeasy Mini Kit (Qiagen, Valencia, Calif.) and following the manufacturer's instructions. The genomic DNA was removed by DNase I treatment. Total RNA (1 μg) was subjected to first-strand cDNA synthesis using the Superscript First-Strand Synthesis System for RT-PCR (Invitrogen, Carlsbad, Calif.). cDNA was subjected to real-time RT-PCR. The expression of POMC mRNA was detected using a POMC gene-specific primer pair and probe (TaqMan Gene Expression Assay, Rn00595020_ml; Applied Biosystems; Foster City, Calif.). PCR amplifications were performed by incubating at 50° C. for 2 minutes and then 950 C for 10 minutes followed by 40 cycles at 95° C. for 15 seconds and 60° C. for 1 minute. The relative quantity of mRNA was calculated by relating the PCR threshold cycle obtained from the tested samples to relative standard curves degenerated from a serial dilution of cDNA prepared from the total RNA. The POMC mRNA level in each sample was normalized with the level of GAPDH mRNA which was measured by a control reagent (PerkinElmer Applied Biosystems).

### Immune Cell Response to NSC-BEP Challenge

NK cell respond to an immune challenge after 3 weeks following PVN transplants of NSC-BEP cells or control cortical cells in 40 days old alcohol-fed, ad lib-fed and pair-fed female rats. The NK cell response was determined by measuring the NK cell cytolytic activity in the PBMC and spleen, and cytokines (IFN-gamma, TNF-alpha) levels in the plasma.

### Tissue and Plasma Sample Collection for NK Assay

At the end of the experiment, animals were decapitated and spleens were obtained aseptically. Splenocytes were isolated from whole spleens of these rats and processed for isolation of NK cells by magnetic separation using negative selection procedures as described previously (Dokur et al. (2005) J. Neuroimmunology 166:29-33; Arjona et al. (2006) Alcohol Clin. Exp. Res., 30:1039-1044). Cells in the negative fraction are enriched NK cells. Cells in positive selection are non-NK-splenocytes. The viability of enriched NK cells range above 90-95%. For PBMC isolation, blood was centrifuged at 1500 g for 20 minutes to remove the plasma. The cell pellet was resuspended in Hanks' balanced salt solution (Gibco BRL/Invitrogen, Carlsbad, Calif., USA) in a volume of the same as the original volume of the centrifuged sample and the cell suspension was carefully layered over the top of 5 ml of 95% Ficoll (Amersham Pharmacia) in a 15 ml Falcon tube. The tubes were centrifuged for 40 minutes at 1500 g and the white cell layer was collected using a Pasteur pipette. PBMCs were rinsed with cold Hanks' balanced salt solution and used for determination of NK cell cytolytic activity.

### NK Cell Cytolytic Assay

Cytolytic activity was determined from quadruplicate measures of effector to target ratios of 200:1, 100:1, 50:1 and 25:1 against chromium-labeled YAC-I lymphoma cells in a 4-hour assay as previously described (Boyadjieva et al. (2001) J. Immunol., 167:5645-5652). The lytic unit was calculated from the cytolytic data at 10% cytolytic activity for 10⁶ effector cells according to Pross et al. (1981) J. Clin. Immunol., 1:51-63. Data will also be expressed as lytic units/NK cell determined by flow cytometry. Expression of the data in this way will allows for the interpretation of whether the absolute lytic activity of the NK cells is modulated or whether changes in activity are due to alteration in the number of NK cells.

### NK Cell Cytolytic Factors and Cytokine Production

Cellular content of granzyme B, perforin, IFN-gamma and TNF-alpha levels were assayed by western blot as described previously (Mona et al. (2004) J. Immunol., 172:2811-2817; Dokur et al. (2003) Alcohol. Clin. Exp. Res., 27:670-676). Plasma and media contents of INF-gamma were measured by ELISA (Amersham Biosciences; Piscataway, N.J.). Relative protein levels were calculated as a percentage of the maximum value observed in each blot. These assays are routinely used in the laboratory (Arjona et al. (2004) J. Immunol., 172:2811-2817; Dokur et al. (2003) Alcohol. Clin. Exp. Res., 27:670-676).

### NSC-BEP Cell Transplants and the Growth of Prostate Tumors Induced by Carcinogen and Hormone in Rats

A study was performed to determine the effects of the cell transplants on carcinogen and hormone induced prostate tumor growth. Carcinogen and hormone exposure was carried out using the well-established MNU and testosterone treatment protocols published previously (Arunkumar et al. (2006) Biol. Pharm. Bull., 29:375-379). In this experiment, control fed male rats were transplanted with NSC-BEP cells, cortical cells or NSC cells in both PVN and then were injected interperitoneally (i.p.) with cyproterone acetate (50 mg/kg body wt.) (Sigma Chemicals) for 21 consecutive days. One day after the last dose of cyproterone acetate, rats received daily i.p. injection of 100 mg testosterone propionate/propylene glycol for 3 days. One day after the last testosterone propionate injection all rats received a single i.v. dose (50 mg/kg body wt.) of MNU (dissolved in saline at 10 mg/ml), through the tail vein. One week after MNU administration, rats received daily i.p. injection of 2 mg/kg body wt. testosterone propionate/kg body wt. for 60 days. After the treatment period rats were killed, prostate was removed from the adhering connective tissue, washed several times with physiological saline, weighed accurately, fixed with 10% neutral buffered formalin and stained with hematoxyline and eosin for determination of tissue histopathology.

### Statistics

The means±standard errors of the data were determined and are presented in the text and figures. Data were analyzed using one-way ANOVA. The differences between groups were determined using the Student-Newmann-Keuls test. A value of p<0.05 was considered significant.

### Results

cAMP-Elevating Agents Increased Differentiation of Hypothalamic NSCs to β-Endorphin Neurons in Culture

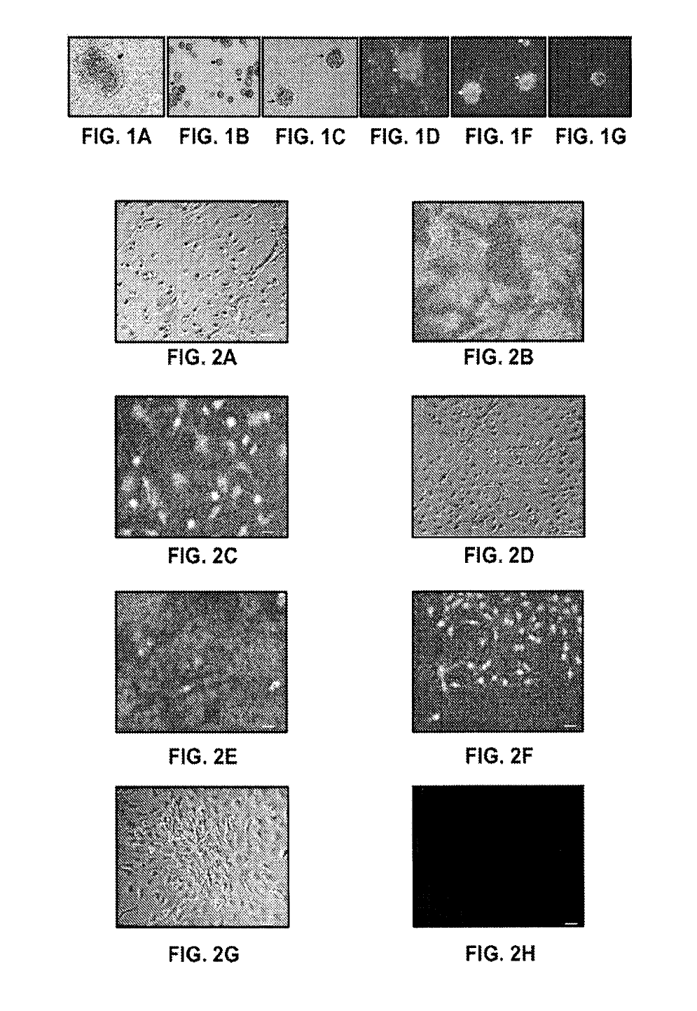

To begin to examine the capacity of NSCs to generate β-endorphin neurons, neurons were purified from embryonic hypothalamic tissues and neurospheres were grown in cultures using stem cells maintaining medium. These neurospheres were maintained in cultures for a period of 2 weeks in the presence or absence of added factors including basic fibroblast growth factor (bFGF). Upon dissociation, they developed secondary neurospheres and formed aggregates that expressed nestin and vimentin (FIG. 1), protein markers of the immature uncommitted phenotype (Lendahl et al. (1990) Cell 60:585-595; Shaw, G. (1998) Neurofilaments. Springer-Verlag, Berlin). The neurosphere was considered NSC. These neurospheres can be maintained in culture for several months by regularly changing medium and splitting cells.

Whether PACAP and dbcAMP affect differentiation of these NSCs into neurons in serum-free neuronal cell-maintaining medium was determined. Within a period of 3 days, many neurospheres start forming single cell with various shapes (FIG. 2A), many of these cells showed significant amount of vimentin (FIG. 2B), and α-internexin immunoreactivity (FIG. 2C) which is a marker of early neuronal phenotypes (Kaplan et al. (1990) J. Neurosci., 10:2735-2748; Carden et al. (1987) J. Neurosci., 7:3489-3504). After 1 week of PACAP and dbcAMP treatments, NSCs began to show filamentous structures (FIG. 2D) and to express neurofilament (NF)-M (FIG. 2E), a neuronal marker (Carden et al. (1987) J. Neurosci., 7:3489-3504), indicating that the neuronal progenitor cells had begun to differentiate into neuronal phenotypes by this time. Characterization of the neuronal phenotypes by the immunohistochemical method revealed that many of these cells were expressing β-endorphin (FIG. 2F). Neuronal stem cells that were maintained in neuronal-cell maintaining media without PACAP and dbcAMP did not show many cells with filamentous structures (FIG. 2G) nor did they show any β-endorphin-staining (FIG. 2H), suggesting the possibility that the cAMP activating agents are necessary for NSCs differentiation to β-endorphin neurons.

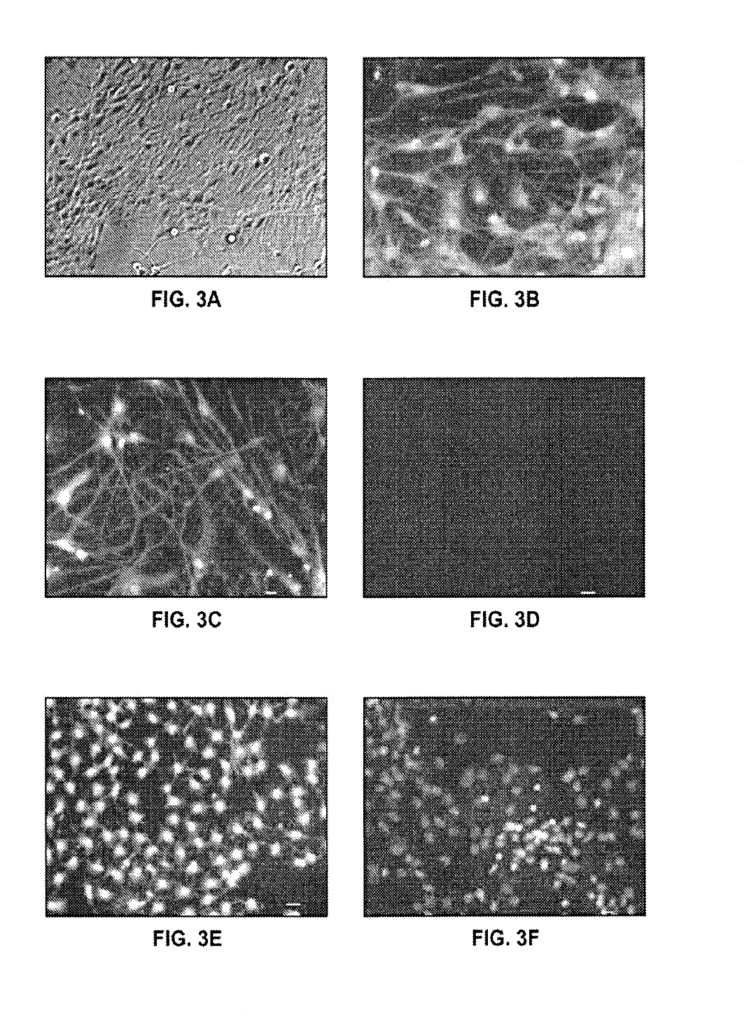

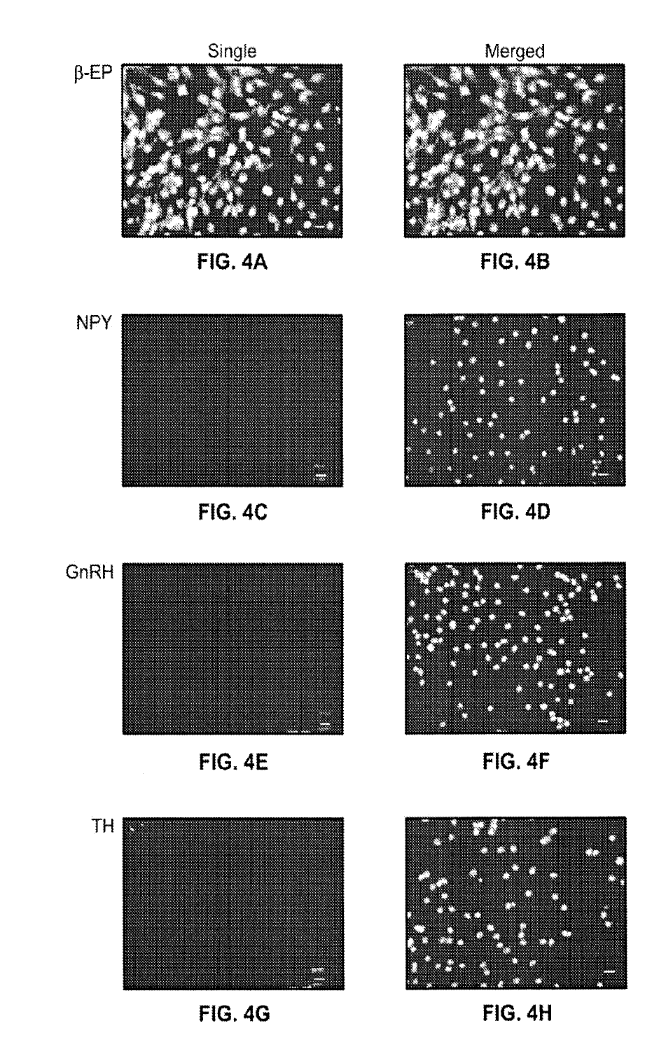

These NSCs were further maintained in serum-free defined neuronal cell culture medium without cell differentiating factors for a period of 1 week in order to determine the permanency of the PACAP/dbcAMP effects on NSC differentiation. By the end of this treatment, all of these cells had neuron-like appearance (FIG. 3A) and expressed neuronal markers (Evans et al. (2002) J. Neurophysiol., 87:1076-1085) MAP2 (FIG. 3B) and type III β-tubulin (FIG. 3C) but not astrocyte cell marker (Raju et al. (1981) Dev. Biol., 85:344-357) glial fibrillary acid protein (GFAP; FIG. 3D), suggesting that all NSCs were now differentiated into neurons. These cells also stained for BEP (FIG. 3E). Control experiment with excess antigen verified BEP immunostaining on differentiated NSCs (FIG. 3F). The BEP antibody used also stained cells producing this peptide in the hypothalamus (FIG. 4A). Further characterization of the differentiated NSCs revealed that 100 percent of these cells are BEP immunopositive (FIG. 4B; note that all the cells identified by the blue color nuclear staining with DAPI also stained for red color BEP). However, these differentiated NSCs did not stain for neuropeptide Y (NPY; FIGS. 4C and D), gonadotropin hormone-releasing hormone (GnRH; FIGS. 4E and F), or tyrosine hydroxylase (TH; an enzyme produces catecholamine including dopamine; FIGS. 4G and H). These are some of the major peptides in the hypothalamus that are positively regulated by the cAMP and PACAP system (Li et al. (1996) Brain Res. Mol. Brain. Res., 41:157-162; Olcese et al. (1997) J. Neuroendocrinol., 9:937-943; Hansel et al. (2001) J. Neurosci. Res., 31:412-418; Mizuno et al. (1998) J. Neuroendocrinol., 10:611-616; Reglodi et al. (2004) Behav. Brain Res., 151:303-312). Hence, activation of the cAMP system leads to differentiation of NSCs to primarily BEP neurons.

cAMP Agent-Induced Differentiated Neurons had β-Endorphin Neuronal Function

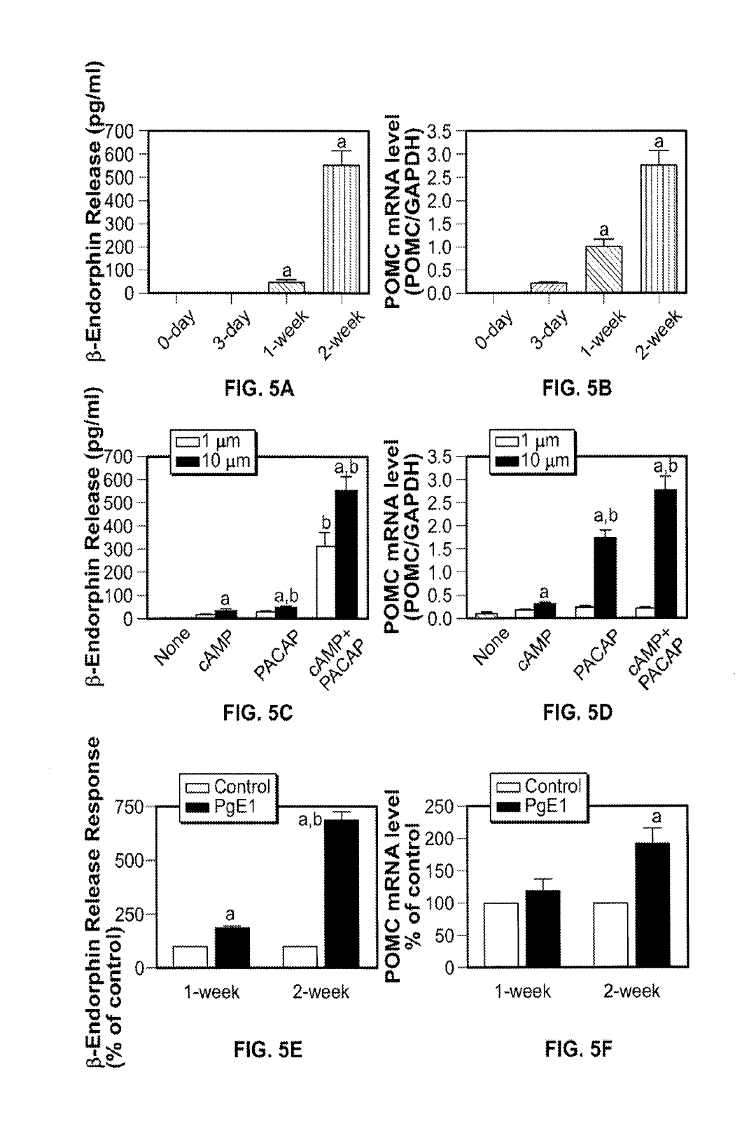

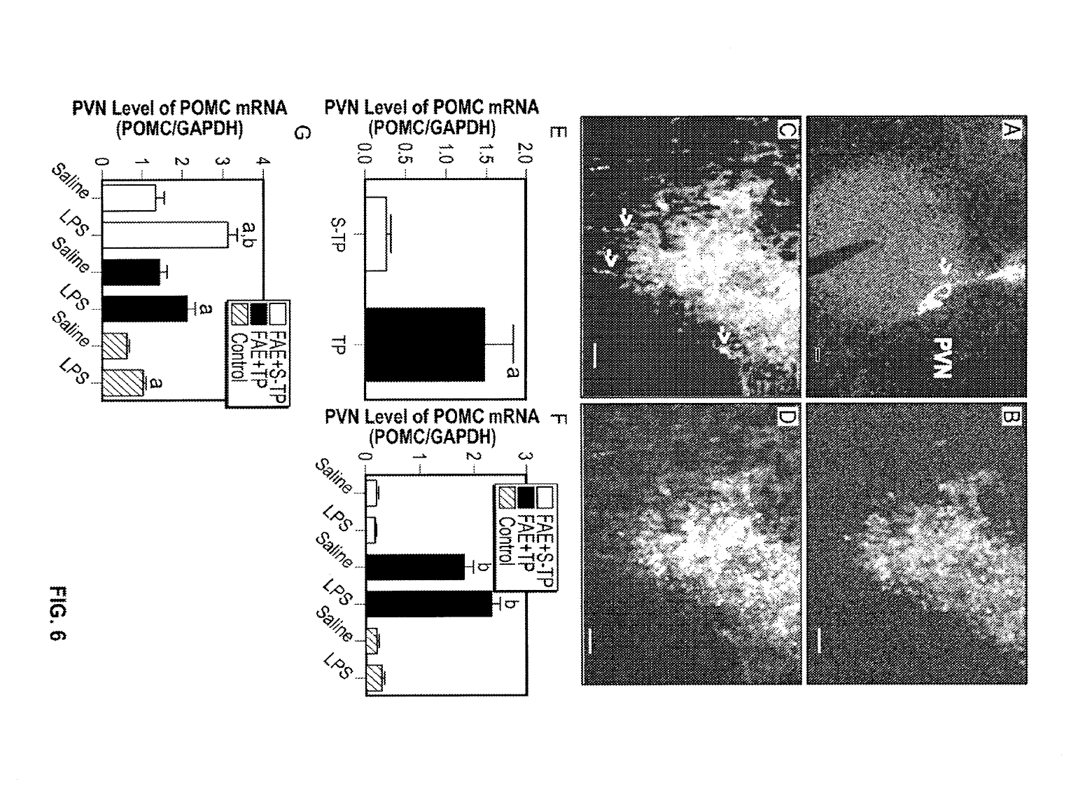

To clarify whether the immunoreactive characteristic of differentiated neuronal progenitor cells reflect their functions, the dynamic of basal secretion of β-endorphin was studied during the period of differentiation in culture. In agreement with the immunohistochemical data, FIG. 5A shows that NSCs at the end of PACAP and dbcAMP treatment at 1 week secreted moderate amounts of BEP in the media, but secreted 10-12-fold larger amounts of the peptide in the media even in the absence of the cAMP elevating agents at 2 weeks. Additionally, the amount of BEP released from these cells showed dose-dependency and additive effects of PACAP and dbcAMP (FIG. 5C). The control cells, which were not treated with PACAP or dbcAMP (0 day), showed no detectable amount of BEP release.

The peptide BEP is processed from a precursor protein proopiomelanocortin (POMC) from arcuate neurons in vivo (Castro et al. (1997) Crit. Rev. Neurobiol., 11:35-37). Whether the in vitro differentiated BEP neurons express POMC mRNA in a fashion similar to that of BEP release was determined. The expression patterns of POMC mRNA levels showed time-dependency and dose-dependency on PACAP and/or dbcAMP and resembled those patterns of BEP release during differentiation (FIGS. 5B and 5D). Together these data indicate that cAMP agents promoted differentiation of NSCs into BEP-producing cells.

After determining that in vitro differentiated neurons express POMC and release BEP in medium, their ability to respond to the neuromodulator-like prostaglandin E1 (PGE1) was studied, which is known to elevate BEP release from hypothalamic cells (Boyadjieva et al. (1997) Alcohol Clin. Exp. Res., 21:1005-1009). FIGS. 5E and F show that, at 1 week, PGE1 only moderately enhanced BEP release and produced no effect on POMC expression from the group of cells that differentiate in the presence of the combination of PACAP and dbcAMP. However, at 2 weeks the BEP-release response and POMC-expression response to PGE1 were significantly increased. These results indicate that the differentiated neurons have the functional capacity to produce and release BEP and to respond to activators such as PGE1.

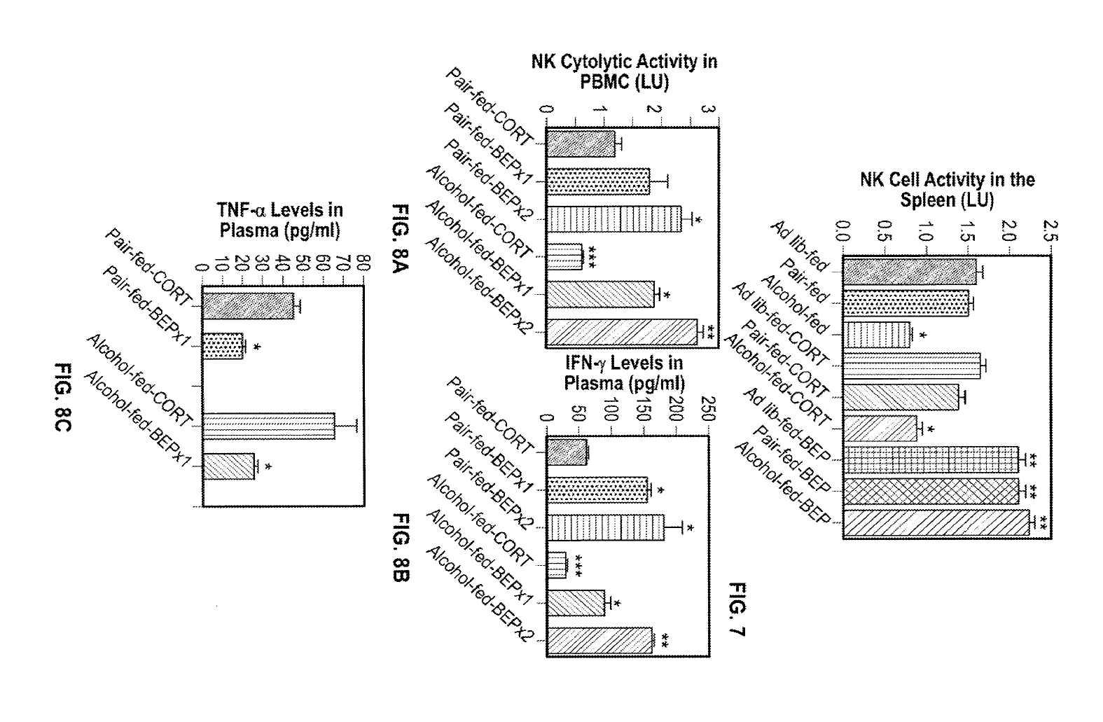

To determine whether the differentiated NSCs maintain their neuronal phenotype in vivo, these cells were labeled with bromodeoxyuridine (BrdU) and transplanted these cells into one of the lobe of PVN of the hypothalamus that contains very few BEP cell bodies. Two weeks after transplantation, these cells remained at the site of transplantation in the PVN (FIGS. 6A and 6B) and showed immunostaining for BEP (FIGS. 6C and 6D). By determining levels of POMC mRNA in the PVN, it was found that expression of this gene was higher by a factor of 6 in the lobe of PVN where the differentiated cells were transplanted than in the contralateral lobe of the PVN that contained sham-transplants (FIG. 6E).

The availability of functional BEP neurons from NSCs led to a replacement therapy study to determine the effect of NSC-BEP neurons on CRH neuronal response in male rats exposed to ethanol during the prenatal period (see Arjona et al. (2006) Alcohol Clin. Exp. Res., 30:1039-1044, for methods of animal preparation). Rats exposed to ethanol during the prenatal period are known to demonstrate CRH hyper-responsiveness to an immune challenge (Lee et al. (2000) Mol. Cell Neurosci., 16:515-528; Taylor et al. (1988) Adv. Exp. Med. Biol., 245:311-317). CRH hyperresponsiveness in alcohol-fed rats has shown to be due to low BEP neuronal function (Sarkar et al. (2007) Endocrinology 148:2828-2834). Hence, the CRH neuronal response to these NSC-BEP transplants was investigated by infusing these cells into one PVN lobe and infusing control medium into the other PVN lobe of the alcohol-fed rats. The changes in the expression of POMC and CRH mRNA levels were then compared between the two sides of the PVN of each animal following systemic administration of LPS. As shown in FIG. 6F, LPS moderately increased the POMC levels in the PVN containing the NSC-BEP neurons but did not affect PVN levels of POMC in the contralateral side without cell transplants in alcohol-fed animals. LPS was also ineffective in altering POMC levels in the PVN of control-fed rats. LPS increased CRH mRNA levels in the PVN of both non-transplanted alcohol-fed and control-fed rats, but the magnitude of the CRH response to LPS was higher in alcohol-fed rats (FIG. 6G). LPS increased the level of CRH mRNA in the PVN infused with NSC-BEP neurons or with control media in alcohol-fed rats. However, the response of PVN lobe side containing NSC-BEP neurons was much lower than the response of the PVN side treated with control medium. These results indicate that NSC-BEP cells were able to reduce the CRH hyperresponsiveness to LPS in alcohol-fed rats.

### NSC-BEP Cell Transplants Activate NK Cell Function

It has been shown that administration of BEP peptide into the PVN increases the cytolytic function of splenic NK cells (Boyadjieva et al. (2001) J. Immunol., 167:5645-5652) by suppressing the inhibitory action of CRH on splenic NK cells via sympathetic neurons (Boyadjieva et al. (2006) Alcohol Clin. Exp. Res., 30:1761-1767). Since NSC-BEP cell transplants can prevent CRH function, it was investigated whether NSC-BEP cell transplants activate NK cell function. This was studied by transplanting NSC-BEP cells or control cells in immune-deficient neonatally alcohol-fed male rats (Arjona et al. (2006) Alcohol Clin. Exp. Res., 30:1039-1044; Zhang et al. (2005)) during the adult period in the one PVN or both PVNs in alcohol-fed and control-fed rats. As a control for NSC-BEP cells, cortical cells were used (Noh and Gwag 1997). Neonatally alcohol-fed rats were used because these rats are immune-deficient and show reduced NK cell function (Arjona et al. (2006) Alcohol Clin. Exp. Res., 30:1039-1044).

Determination of NK cell functions revealed that NSC-BEP transplants significantly increased NK cell cytolytic activity in the spleens (FIG. 7) in control-fed and alcohol-fed rats. The NK cell activation effect of NSC-BEP cells was higher when transplanted in both PVNs as compared to only one PVN. Bilateral NSC-BEP cell transplants in both PVNs were also able to increase cytolytic function of NK cells derived from peripheral blood mononuclear cells (PBMC; FIG. 8A) and levels of IFN-gamma in peripheral plasma (FIG. 8B) of alcohol-fed and control-fed animals. However, NSC-BEP cell transplants decreased TNF-alpha levels in the plasma of alcohol-fed and control-fed animals (FIG. 8C). The levels of both basal and LPS-induced NK cytolytic function and IFN-gamma mRNA levels in splenic tissues of alcohol-fed animals transplanted with NSC-BEP neurons in the PVN were higher than those levels in alcohol-fed animals with sham transplants in the PVN. These results indicate that NSC-BEP cell transplants are effective in activating NK cell functions.

### NSC-BEP Cell Transplants Reduce the Growth of Prostate Tumors Induced by Carcinogen and Hormone in Rats

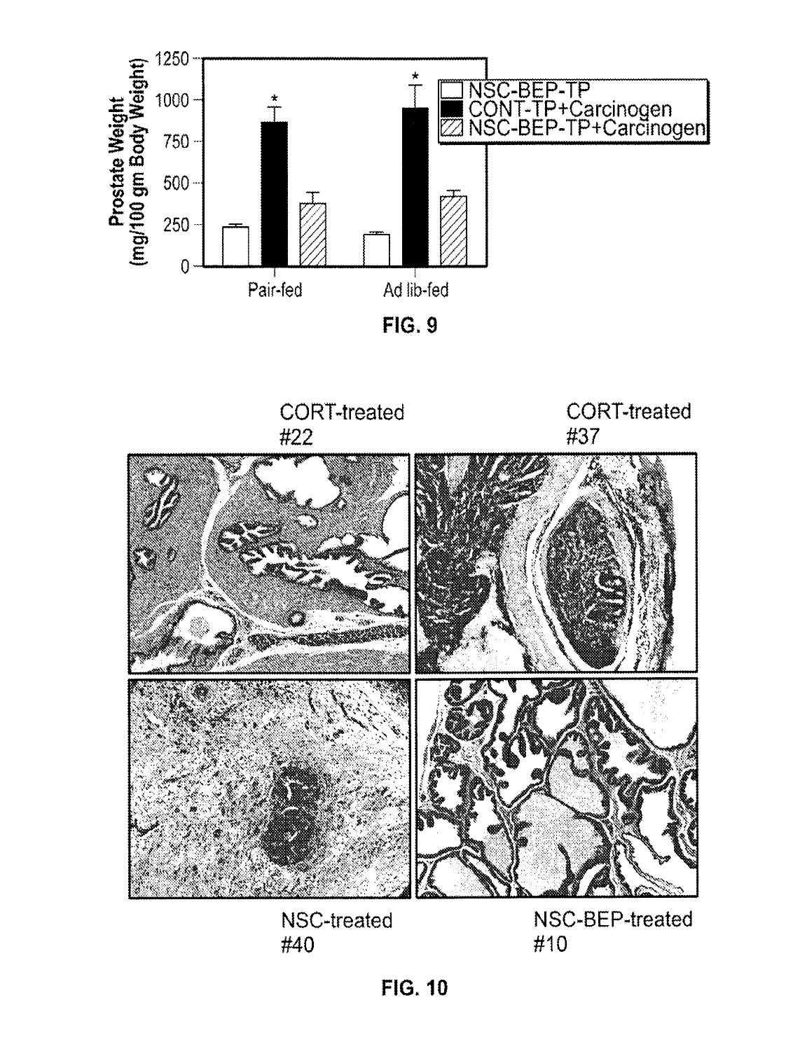

An experiment was conducted to determine the effects of the NSC-BEP cell transplants on carcinogen and hormone induced prostate tumor growth using well-established MNU and testosterone treatment protocols (Arunkumar et al. (2006) Biol. Pharm. Bull., 29:375-379). In this experiment, control fed male rats were transplanted with NSC-BEP cells, cortical cells or NSC cells in both PVN and then were treated with carcinogen and hormones. After the treatment period, rats were killed; prostate was removed from the adhering connective tissue, washed and weighed. The wet weights of prostate/kg of body weight were 3-4-fold lower in animals treated with NSC-BEP cells than in cortical cells or NSC cells (FIG. 9). A limited characterization of histopathology revealed substantial neoplasia in all of the prostrates of four control cell transplanted animals but no neoplasia in the prostate of four NSC-BEP cells transplanted animals (FIG. 10). This data indicates that NSC-BEP cell therapy can prevent tumor cell growth.

### Discussion

One finding was that neuronal progenitor cells can be generated from the rat embryonic hypothalamic tissues and propagated by cAMP-elevating agents to produce BEP neurons in cultures. Like in vivo, these cells go on to produce and secrete BEP and respond positively to the neuromodulator challenge. Early in mammalian brain development, NSCs express vimentin and nestin intermediate filament proteins (Lendahl et al. (1990) Cell 60:585-595; Shaw, G. (1998) Neurofilaments, Springer-Verlag, Berlin). As development progresses, these cells divide and differentiate to produce neuronal or glial lineage. Here it was shown that cultures derived from embryonic rat hypothalamic cells generated nestin-positive neuronal progenitor cells. These cells grew and generated secondary nestin-positive spheres when primary spheres were mechanically dissociated and cultured under the same experimental conditions.

bFGF has previously been shown to induce or enhance the proliferation of neurospheres and neuronal stem cells (Vescovi et al. (1993) Neuron 11:951-966). Herein, bFGF was used to generate and develop a colony of undifferentiated cells, which expressed nestin, or vimentin. Here it is shown that the NSCs isolated in vitro from rat embryonic hypothalamic neurons responded to bFGF under serum-free conditions to give rise to clonal aggregates of undifferentiated neurospheres. The differentiation of hypothalamic-derived neuronal precursor cells to neurons was initiated by 3 days after PACAP and/or dbcAMP treatments since they expressed the marker for immature neurons-α-internexin (Kaplan et al. (1990) J. Neurosci., 10:2735-2748; Carden et al. (1987) J. Neurosci., 7:3489-3504). These cells differentiated into neuronal phenotypes by the end of a week of PACAP and/or dbcAMP treatments since they expressed NF-M. This neurofilament protein is a well-known neuronal marker for neurodifferentiation (Carden et al. (1987) J. Neurosci., 7:3489-3504). In addition, 1 week of post PACAP and cAMP treatments, these treated cells expressed the neuronal markers MAP2 and type III β-tubulin but not the astrocyte marker GFAP (Evans et al. (2002) J. Neurophy., 87:1076-1085; Raju et al. (1981) Dev. Biol., 85:344-357). This is consistent with the conclusion that combination treatments of PACAP and dbcAMP activated the development and maturation of precursor hypothalamic spheres to the pure population of neurons.

PACAP belongs to a peptide family that includes secretin, glucagons, growth hormone-releasing factor, and vasoactive-intestinal peptide (Sherwood et al. (2000) Endocr. Rev., 21:619-670). Recent data suggest that PACAP exerts developmental actions. PACAP gene expression and PACAP immunoreactivity are widely distributed in neurons within the embryonic and neonatal rat brain (Nielsen et al. (1998) J. Comp. Neurol., 394:403-415). Activation of PACAP receptors regulates the proliferation of the developing neuroblasts in vitro and in vivo (Lee et al. (2001) Mol. Cell Neurosci., 16:515-528; DiCicco-Bloom et al. (2000) Dev. Bio., 219:197-213). PACAP and its receptors are expressed in embryonic neural tubes, where they appear to regulate neurogenesis (Lee et al. (2001) Mol. Cell Neurosci., 16:515-528). The instant results demonstrated that PACAP promoted the differentiation of embryonic stem cell neurons. The results reported here also provide the first evidence that dbcAMP interacts with PACAP in the induction of differentiated mature neurons. Moreover, the results here demonstrated for the first time that the combination of PACAP and dbcAMP promote differentiation and functional maturation of BEP neurons.

It has been shown that dbcAMP acts as a neurotropic factor for immature BEP neurons (De et al. (1994) J. Biol. Chem., 269:26697-26705). Also, studies of transduction pathways identified that the adenyl cyclase-cAMP system is an important second messenger system in the regulation of hormone secretion and POMC gene regulation (Lundblad et al. (1998) Endocr. Rev., 9:135-158). Since PACAP is a potent inducer of cellular levels of cAMP in developing neurons (DiCicco-Bloom et al. (2000) Dev. Bio., 219:197-213), the interactive actions between this peptide and dbcAMP it was observed that the cAMP signaling system regulates' early differentiation of BEP neurons.

The availability of functional BEP neurons from NSCs also is useful for replacement therapy study. It was found that the NSC-derived BEP neurons have the ability to produce POMC for a minimum period of two weeks after intra-PVN transplantation. Exogenous BEP has been shown to inhibit CRH neuronal function (Boyadjieva et al. (2006) Alcohol Clin. Exp. Res., 30:1761-1767; Plotsky et al. (1986) J. Endocrinol., 3:1). BEP and CRH are known to regulate immune functions (Boyadjieva et al. (2001) J. Immunol., 167:5645-5652; Boyadjieva et al. (2006) Alcohol Clin. Exp. Res., 30:1761-1767; Irwin et al. (1988) Am. J. Physiol., 255:R744-747), and these peptides and their mRNA levels are increased following LPS treatments or other immune challenges (Chen et al. (2006) Alcohol Clin. Exp. Res., 30:1925-1932; Lee et al. (2000) Mol. Cell Neurosci., 16:515-528; Taylor et al. (1988) Adv. Exp. Med. Biol., 245:311-317). In this study, it is shown that the level of POMC mRNA in BEP neuronal transplants was moderately, but not significantly, increased following the LPS challenge. However, even in this moderate elevation of POMC, the CRH mRNA response to LPS was markedly decreased in animals with the BEP neuronal transplant in the PVN. Hence, the NSC-derived BEP neurons are reducing the CRH neuronal ability to respond to the LPS challenge. These results indicate that NSC-derived BEP neurons maintain functionality when they are transplanted in vivo.

Since NSC-BEP cell transplants can prevent CRH function, it was investigated whether NSC-BEP cell transplants activate NK cell function. The NK cell activation effect of NSC-BEP cells was higher when transplanted in both PVNs as compared to only one PVN. Bilateral NSC-BEP cell transplants in both PVNs were also able to increase NK cytolytic function and plasma levels of IFN-gamma in alcohol-fed animals. However, NSC-BEP cell transplants decreased TNF-alpha levels in the plasma of alcohol-fed and control-fed animals. The levels of both basal and LPS-induced IFN-gamma mRNA levels in splenic tissues of alcohol-fed animals transplanted with NSC-BEP neurons in the PVN were higher than those levels in alcohol-fed animals with sham transplants in the PVN. However, both the basal and LPS-induced TNF-alpha mRNA were similar in spleen tissues of alcohol-fed animals transplanted with or without NSC-BEP neurons. These results indicate that NSC-BEP cell transplants are effective in activating NK cell functions.

The ability of NSC-BEP to increase the NK cell activity was correlated with the ability of this cell transplant to inhibit prostate tumor growth and progression. These data indicate that NSC-BEP cell therapy can increase the body's innate immunity to prevent growth and progression of cancer cells.

Pituitary adenylate cyclase-activating peptide (PACAP), a cAMP-activating agent, is highly expressed in the hypothalamus during the period when many neuroendocrine cells become differentiated from the neural stem cells (NSCs). Activation of the cAMP system in rat hypothalamic NSCs differentiated these cells into beta-endorphin (BEP)-producing neurons in culture. When these in vitro differentiated neurons were transplanted into the paraventricular nucleus (PVN) of the hypothalamus of an adult rat, they integrated well with the surrounding cells and produced BEP and its precursor gene product, proopiomelanocortin (POMC). Animals with BEP cell transplants demonstrated remarkable protection against carcinogen induction of prostate cancer. Unlike carcinogen-treated animals with control cell transplants, rats with BEP cell transplants showed rare development of glandular hyperplasia, prostatic intraepithelial neoplasia (PIN) or well-differentiated adenocarcinoma with invasion following N-methyl-N-nitrosourea (MNU) and testosterone treatments. Rats with the BEP neuron transplants showed increased natural killer (NK) cell cytolytic function in the spleens and peripheral blood mononuclear cells (PBMC), elevated levels of antiinflammatory cytokine interferon-gamma IFN-gamma and decreased levels of inflammatory cytokine tumor necrosis factor-alpha (TNF-alpha) in plasma. These results identify a critical role for cAMP in the differentiation of BEP neurons and revealed a novel role of these neurons in combating the growth and progression of neoplastic conditions like prostate cancer, possibly by increasing the innate immune function and reducing the inflammatory milieu.

### Example 2

### Materials and Methods

Preparation of Neurospheres from the Fetal Rat Hypothalamus

Mediobasal hypothalamic tissues from fetal rats (embryonic day 17) of the Sprague Dawley (SD) strain (Charles River Laboratories, Wilmington, Mass.) were dissociated by mechanical dispersion and neurospheres were prepared as described in Example 1.

### Immunohistochemical Characterization of NSCs

Immunohistochemistry was performed as described in Example 1. Expression levels of POMC mRNA in stem cells and differentiated cells were assayed by a quantitative RT-PCR (qRT-PCR) on an ABI PRISM 7700 Sequence Detector (Perkin Elmer Applied Biosystems, Foster City, Calif.), as described previously (Chen et al. (2004) J. Neurochem., 88:1547-1554). The immunoreactive BEP levels in culture media were measured by a radioimmunoassay (RIA; De et al. (1994) J. Biol. Chem., 269:26697-26705).

### Animal Preparation and Surgery

Pregnant female Sprague Dawley rats were obtained from Charles River Laboratories (Wilmington, Mass.) and housed in a controlled environment and provided rodent chow meal and water ad libitum as described in Example 1. Male pups of these dams were used in these series of studies.

In vitro differentiated BEP cells were dissociated using 0.05% trypsin/EDTA, washed and resuspended in HDMEM and SS medium for transplantation. Cortical cells were prepared (Noh et al. (1997) Exp. Neurol., 146:604-608) and maintained in cultures for 4 days, trypsinized, and resuspended in HDMEM and SS medium for transplantation. Cells for transplantation were prepared as described in Example 1. Male rats between 35-60 days of age were anesthetized and injected with 1.0 μl of cell suspension (20,000 cells/lobe) as described in Example 1.

### NSC-BEP Cell Transplants Viability

A group of male rats with differentiated BEP cell transplant in one of the PVN lobe was used for detection of BEP immunoreactivity in the transplanted cells. These animals were anesthetized with sodium pentobarbital and perfused with 0.1 M PBS followed by 4% paraformaldehyde in PBS, post-fixed, frozen in cryoprotectant, serially sectioned (40 μm) and double-stained for BrdU and BEP using immunohistochemistry methods. A separate group of rats with the differentiated BEP cell transplant or non-viable BEP cells transplant in one of the PVN lobe were used for measurement of POMC mRNA using the qRT-PCR analysis and BEP levels in the PVN and plasma using RIA.

### NSC-BEP Cell Transplants and the Growth of Prostate Tumors Induced by MNU and Testosterone in Rats

The effects of the BEP cell transplants on MNU and testosterone-induced prostate tumor growth were determined as described previously. Adult male rats were transplanted with BEP cells or cortical cells into both PVN at 90 days of age and then were injected i.p. with cyproterone acetate (50 mg/0.3 ml DMSO/kg; Sigma) for 21 consecutive days followed by daily i.p. injections of 100 mg/kg testosterone propionate (Steraloids, Inc., Newport, R.I.) in propylene glycol for 3 days. One day after the last testosterone injection all rats received a single i.p. dose (50 mg/kg bw) of MNU (Sigma) dissolved in saline at 10 mg/ml. One week after MNU administration, rats received daily i.p. injection of testosterone (2 mg/kg bw) for 60 days. After this treatment period, rats were injected i.p. with 0.3 ml saline or LPS (100 μg/ml saline/kg), and 3 hours later they were sacrificed, trunk blood was collected for the corticosterone ELISA assay (Diagnostic System Laboratories, Webster, Tex.). Prostates were removed from the adhering connective tissue, washed several times with physiological saline, weighed, fixed with 10% neutral buffered formalin and stained with hematoxylin and eosin for determination of tissue histopathology.

### Innate Immune System Response to BEP Cell Transplants

The immune system response to BEP cell transplants was determined by measuring the NK cell cytolytic activity in the spleen and PBMC and cytokine (IFN-gamma and TNF-alpha) levels in the plasma following 4 weeks of transplantation with BEP cells or cortical cells into one or both PVNs in male rats at 60-70 days of age. At the end of the experiment, these rats were decapitated and the spleens and peripheral blood were obtained and used for isolation of splenocytes and PBMC to measure NK cell cytolytic activity as described previously (Boyadjieva et al. (2006) Alcohol Clin. Exp. Res., 30:1761-1767). Plasma levels of INF-gamma and TNF-alpha were measured by ELISA (Amersham Biosciences, Piscataway, N.J.).

### Statistics

Means±standard errors of the data calculated and presented in the text. The significance of the differences between two experimental groups was analyzed by the t test. Multiple groups of data were analyzed using one-way analysis of variance. The differences between groups were determined using the Student-Newmann-Keuls test. The proportions of tumors of each type developed between treatments groups were compared using the Fisher's exact test. A value of P<0.05 was considered significant.

### Results

cAMP-Elevating Agents Increased Differentiation of Hypothalamic NSCs to BEP Neurons in Cultures

To examine the capacity of NSCs to generate BEP neurons, neurons were purified from embryonic hypothalamic tissues and grew neurospheres in cultures using stem cell-maintaining medium. It was determined whether or not PACAP and dbcAMP differentiate NSCs into neurons. An initial screening of the response of various doses (0.1-10 μM) of PACAP and dbcAMP alone revealed a moderate effect of these agents on neurosphere differentiation, since many cells remained as neurosphere like structures. However, using a combined treatment of 10 μM concentrations of PACAP and dbcAMP, many neurospheres started forming single cells with various shapes within a 3-day period. Many of these cells expressed significant amounts of vimentin and α-internexin immunoreactivity, which are markers of early neuronal phenotypes. After 1 week of PACAP and cAMP treatment, NSCs began to show filamentous structures and to express neurofilament (NF)-M, a neuronal marker, indicating that the NSCs had begun to differentiate into neurons. Determination of the phenotype revealed that many of these cells (50-60%) were expressing BEP at this stage of differentiation. NSCs that were not treated with PACAP and cAMP showed no staining for BEP (IH).

The differentiated NSCs were further maintained in a defined-neuronal cell culture medium for a period of 1 week in order to determine the permanency of the PACAP/cAMP effects. By the end of this treatment, all of these cells had a neuron-like appearance, and they expressed neuronal markers MAP2 and type III β-tubulin, but not the astrocyte cell marker GFAP, suggesting that all NSCs were now differentiated into neurons. These cells also stained for BEP. A control experiment with excess antigen verified the specificity of BEP immunostaining in differentiated NSCs (S1A). The staining of BEP for these cells merged well with the nuclear staining for DAPI (S1B), suggesting that the majority of NSCs is differentiated into BEP cells. However, these differentiated NSCs did not stain for neuropeptide Y, gonadotropin hormone-releasing hormone or tyrosine hydroxylase (S1C-E). These are some of the major peptides in the hypothalamus positively regulated by the cAMP and PACAP system. Hence, activation of the cAMP system result in differentiation of NSCs to primarily BEP neurons.

cAMP Agent-Induced Differentiated Neurons Produced and Released BEP

To clarify whether the differentiated NSCs replicate the functions of BEP neurons, the dynamics of basal secretion of BEP was studied during the period that the NSCs were differentiating in culture as well as after the completion of differentiation. In agreement with the immunohistochemical data, results showed immediately after the 1 week treatment with PACAP and cAMP, NSCs secreted moderate amounts of BEP into the media. Furthermore, 1 week after the weeklong treatment with PACAP/cAMP, NSCs secreted an amount of peptide in the media 10-12-fold greater than that which was produced immediately after the treatment. Like BEP release, the POMC mRNA levels in the cells were markedly increased at this time. Additionally, the amount of hormone released from these cells by PACAP and cAMP as a result of the amount of BEP neuron differentiation showed dose-dependency and synergistic effects when the two differentiating agents were combined. The expression patterns of POMC mRNA resembled the patterns of BEP released during PACAP and cAMP-induced differentiation. The ability of differentiated BEP neurons to respond to prostaglandin E1 (PGE1) was also studied, which is known to elevate BEP release from hypothalamic cells. Results show that BEP release and POMC-expression of the differentiated neurons were increased by PGE1. These results indicate that the differentiated neurons produce and release BEP in culture.

To determine whether the differentiated neurons maintained their neuronal phenotype in vivo, they were labeled with bromodeoxyuridine (BrdU) and transplanted into one lobe of the PVN of the hypothalamus, a site containing very few BEP cell bodies. Two weeks after the transplantation, these cells remained at the site of transplantation in the PVN and showed immunostaining for BEP. By determining levels of POMC mRNA in the PVN, it was found that the expression of this gene was higher by a factor of 6 in the PVN where the BEP cells were transplanted than the lobe of the PVN into which nonviable BEP cells had been implanted. Determination of BEP protein levels in the hypothalamus and in plasma revealed that the transplants increased levels of this protein in the site of transplants but not in the circulation.

cAMP Agent-Induced Differentiated BEP Neurons Reduced MNU-Induced Prostate Tumors

To determine the effect of BEP cell transplants on tumor growth and development, BEP cells were implanted in both lobes of the PVN of male rats and treated them with MNU and testosterone as previously described. For the control transplant, viable, non-BEP-producing fetal rat cortical cells were used rather than the nonviable BEP cells for a long-term transplant study. The viability of BEP cell transplant was tested by determining the plasma corticosterone response to LPS in the animal prior to sacrifice. It was hypothesized that if BEP cell transplants were functional they would have the ability to inhibit LPS-induced CRH release and therefore corticosterone release in the circulation. BEP neurons have been shown to inhibit CRH, which regulates the plasma level corticosterone. It was found that LPS increased plasma corticosterone level in rats treated with control transplants and MNU and testosterone (saline-285±15; LPS-478±20; N=11-12; P<0.05) but not in rats treated with BEP cell transplants without MNU and testosterone (saline-305±15; LPS-328±14; N=5-6) or with MNU and testosterone (saline-355±35; LPS-398±20; N=8-9), suggesting that BEP neuron transplants were functional until the end of the treatment.

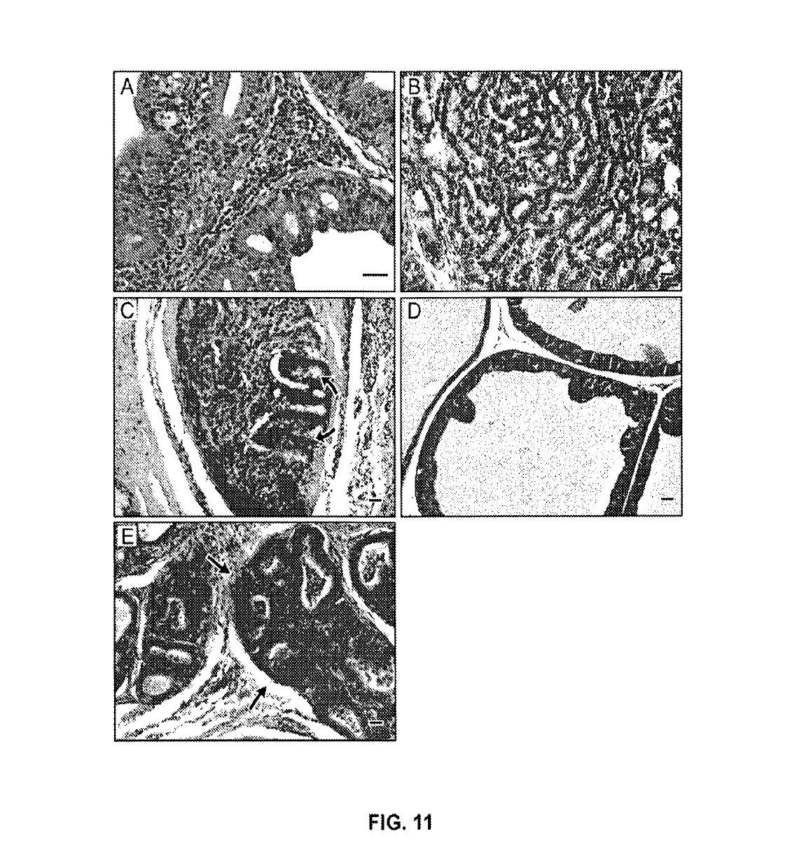

It was found that total weight of prostates in rats treated with BEP neuron transplants and carcinogen did not significantly differ from those in rats treated with BEP neuron transplants and vehicle but differed from those in rats treated with control transplants plus carcinogen (total prostate weight; mg/100 g body weight; BEP neurons+vehicle-213±13; BEP neurons+carcinogen-396±50; CONT+carcinogen-908±109; P<0.001, CONT+carcinogen vs. the rest; N=11-14). The prostates of rats receiving control cell transplants plus carcinogen displayed glandular hyperplasia (FIG. 11A), PIN (FIG. 11B) and occasionally well-differentiated adenocarcinoma with invasion (FIG. 11C). These lesions were primarily localized in the dorsolateral and anterior prostate. Similar to the non-carcinogen-treated controls, rats receiving BEP neuronal transplants and carcinogen showed either normal prostatic morphology (FIG. 11D) or mild epithelial atypia with glandular crowding (FIG. 11E). The incidence of adenocarcinoma was markedly lower (only 1 out of 17 rats examined; P<0.03) in the carcinogen plus BEP neuron transplant group than those rats receiving carcinogen and control cell transplants (22 out of 24 rats examined; Table 1).

TABLE 1
Effect of BEP cell transplants on prostatic neoplasia.

| | % normal | % atyp- | % hyper- | % neopla- |
|---|---|---|---|---|
| Treatment | (n) | ia (n) | plasia (n) | sia (n) |
| BEP cells + Vehicle | 67 (14) | 33 (7) | 0 | 0 |
| CONT + Carcinogen | 3 (1) | 0 | 48 (11)^{a} | 48 (11)^{a} |
| BEP cells + Carcinogen | 65 (11) | 29 (5) | 6 (1) | 0 |

BEP cells or cortical cells (controls; CONT) were transplanted into the PVN of male rats. The rats were then treated with MNU and testosterone or with vehicle.
^{a}P < 0.03, % neoplasia and % hyperplasia in CONT vs. BEP cells as determined by Fisher's exact test.

### BEP Neuronal Transplants Increased NK Cell Cytolytic Function and Altered Production of IFN-Gamma

Since NK cells with potent cytotoxic activity are known to effectively kill prostate cancer cells, it was investigated whether BEP cell transplants altered the NK cell cytolytic function. The effect of BEP cells on inflammatory and anti-inflammatory cytokine levels in circulation was also studied, since this peptide is known to have potent anti-inflammatory effects in the body. Additionally, epidemiologic studies, together with laboratory and clinical studies, suggest that infection and inflammation contribute to the early development of prostate cancer. These issues were investigated by determining whether BEP cells can elevate NK cell function and alter circulatory levels of inflammatory and anti-inflammatory cytokines in rats. To characterize the influence of BEP cell transplants influence on NK cells, the effects of these transplants either in one PVN or in both PVN of rats were determined. Data showed that BEP neuron-induced activation of splenic NK cell cytolytic function was greater when these cells were transplanted in both PVNs as compared to only one PVN. BEP transplants produced similar dose-response effects on NK cell cytolytic activity in PBMC and on IFN-gamma levels in plasma of rats. In contrast, BEP transplants dose-dependently reduced levels of TNF-alpha levels in the plasma of rats. These results suggest that BEP cell transplants are effective in activating NK cell cytolytic functions and reducing the body's inflammatory milieu.

### Discussion

One finding was that under appropriate conditions, NSCs can be generated from rat embryonic hypothalamic tissues and propagated by cAMP-elevating agents to produce BEP neurons in cultures. Like in vivo, these cells go on to produce and secrete BEP, and they respond positively to the neuromodulator challenge. When transplanted in the hypothalamus, BEP cells survive and produce the peptide hormone. These BEP cell transplants inhibit prostate tumor development, possibly by increasing the NK cell activity, reducing the body's inflammatory milieu and by yet unknown immune surveillance mechanisms. These results identify a critical role for cAMP in the differentiation of BEP neurons and reveal a novel role of these neurons in controlling prostate tumor growth.

PACAP belongs to the peptide family that includes secretin, glucagons, growth hormone-releasing factor and vasoactive-intestinal peptide. Recent data suggest that PACAP affects developmental processes. PACAP gene expression and PACAP immunoreactivity are widely distributed in neurons within the neonatal rat brain. Activation of PACAP receptors regulates the proliferation of developing neuroblasts. PACAP and its receptors are expressed in the embryonic neural tube, where they appear to regulate neurogenesis. The activation of PACAP signaling in vitro has been shown to enhance NSC proliferation/survival through a PKA-independent mechanism. In contrast, PACAP has been shown to promote NSC self-renewal and neurogenesis through a mechanism dependent on PKA activation. The instant results demonstrate that PACAP and dbcAMP, which is also an activator of PKA, interact to control the differentiation and functional maturation of BEP neurons.

It has been shown that dbcAMP acts as a neurotropic factor for immature BEP neurons. Also, studies of transduction pathways identified the cAMP system as an important second messenger system in the regulation of hormone secretion and POMC gene regulation. Since PACAP is a potent inducer of cellular levels of cAMP in developing neurons, the interactive actions observed between this peptide and dbcAMP support a role of the cAMP signaling system in the regulation of early differentiation of BEP neurons.

Chronic high levels of stress are known to enhance carcinogenesis in rat models (Spiegel et al. (2001) Semin. Clin. Neuropsychiatry 6:252-265). Furthermore, behavioral interventions aimed at reducing stress and increasing optimism in cancer patients have been documented to enhance immunity and to reduce tumor growth. These studies have identified the importance of stress regulation in the management of cancer growth. The data presented here show that the NSC-derived BEP neurons, when transplanted in the PVN, remain at the site of transplantation and are well integrated with the other cells at this site. They appear to be functional as significant amounts of POMC mRNA and BEP peptide were detected in the tissue containing the transplanted cells but not in the tissue containing control cells. The transplanted ex vivo produced BEP neurons also showed remarkable anti-tumor activity. These data indicate that stress-relieving BEP neurons have the ability to suppress the growth of prostate cancers.

In this study, it was found that ex vivo-produced BEP neuronal transplants significantly increased NK cell cytolytic activity in the spleen and in the PBMC. In addition, the transplants increased IFN-gamma levels in plasma while reducing TNF-alpha levels. These data are in agreement with the finding that administration of BEP peptide in the PVN increases NK cell function. Because the transplants increased BEP levels in the hypothalamus but not in plasma, it is possible that the peptide may have inhibited sympathetic input to the spleen to increase NK cell cytolytic function. The NK cell is a critical component of the innate immune system and plays a central role in host defense against tumor cells. The importance of the NK cell in controlling tumor growth and metastasis of cancer cells has been clearly demonstrated in severe combined immunodeficiency mice. Hence, the possibility arises that the higher level of NK cell cytolytic activity may have caused unfavorable conditions for prostate cancer cell growth. In addition, in the BEP cell-treated animals the lower inflammatory milieu that was achieved by the higher level of anti-inflammatory IFN-gamma and lower level of inflammatory TNF-alpha may have also been involved in inhibiting prostate cancer growth. Proliferative inflammatory atrophy, a prostate cancer precursor lesion, ties the inflammatory response to prostatic carcinogenesis. Somatic epigenetic alterations, present in all prostate cancers, also appear to arise in the setting of inflammation. BEP neuronal transplants inhibit prostate tumor development possibly by increasing the NK cell cytolytic activity and/or ameliorating the inflammation. These data provide strong evidence that hypothalamic BEP neurons play a critical role in controlling tumor growth. Because neuronal differentiation from NSC persists in the adult, the BEP-inducing therapies by cAMP-activating agents can be used as a treatment for cancer.

### Example 3

### Beta-Endorphin Neuron Transplants in the Hypothalamus Reduces Rheumatoid Arthritis Development in a Rat Model

It was shown recently that implantation of stem cell-derived beta-endorphin producing (BEP) neurons into the rat hypothalamus is capable in blocking components of inflammation, including proinflammatory cytokine production in a cancer model. In this study, the effect of the BEP cells transplantation into the paraventricular nucleus of the hypothalamus on the systemic inflammation exemplified by adjuvant-induced arthritis (AIA) is reported, which is a preclinical in vivo model of rheumatoid arthritis. In this model, young female Lewis rats were transplanted with BEP cells or the neuronal cortical cells into the paraventricular nuclei. After the end of the recovery period, AIA was induced by intradermal injection of the oil suspension of Mycobacterium butyricum at the base of the tail. After the onset of the disease, the severity of the AIA in the animals was assessed by measuring ankle circumference, paw thickness and scoring the extent of paw edema (artcular index). The animals were monitored for three weeks after the injection. There were statistically significant reductions in the articular index (P<0.05 at day 12, P<0.02 at day 21; n=6-8) and posterior paw thickness (P<0.05 at day 16, P<0.04 at day 21; n=7-8) in BEP neuron transplanted rats but not in control neuron transplanted rats. Ankle circumferences were also decreased in the BEP-treated cells in comparison to control (P<0.01 at day 21; n=7-8). This study demonstrates for the first time that activation of BEP cell in the rat hypothalamus may protect from rheumatoid arthritis development.

### Example 4

### Neural Stem Cell-Derived Beta-Endorphin Neuron Transplants into the Brain Increase Natural Killer Cell Activity, Decrease Antiinflammatory Cytokines and Prevent Metastatic Colonization in Rats

A review of studies that evaluated psychological factors and outcome in cancer patients suggested an association between certain psychological factors and the growth and metastasis of cancer. It has been suggested that the effects of stress on the immune system may in turn affect the growth of some tumors. It has been shown that a set of hormone secreting nerve cells in the hypothalamus, called beta-endorphin producing (BEP) neurons, plays a role in regulating both the stress response and immune function. Hence, it was tested whether activation of BEP neurons may help inhibit metastatic colonization. To test this rat neural stem cells were differentiated from the hypothalamus into BEP neurons with the aid of cAMP-activating agents in culture, which were later transplanted into the paraventricular nuclei (PVN) of the hypothalamus of live Fischer 344 rats. Control rats were transplanted with cortical cells or not operated. Following 3 weeks after cell transplantation, these rats were inoculated intravenously with rat mamory tumor cells (MADB106 tumor cells) for the assessment of lung tumor retention (LTR). Additionally, the impact of cell transplants on plasma levels of stress hormone corticosterone and cytokines interferon gamma (INF-gamma, tumor necrosis factor alpha (TNF-alpha and interleukin 6 (IL6) in plasma and the levels of natural killer (NK) cell cytotoxicity in peripheral blood and spleen was evaluated. When the in vitro differentiated BEP neurons were transplanted into the PVN of an adult rat, they integrated well with the surrounding cells and produced BEP and it precursor gene proopiomelanocortin. The animal with BEP neuron transplants showed no retention of MADB106 cells in lung or other tissues, whereas the animals with cortical cell transplants or no transplants showed significant retention of these cells and visible surface metastasis in the lungs. Rats with BEP neuron transplants also showed increased NK cell cytolytic function in the spleens and peripheral blood mononuclear cells, elevated levels of anti-inflammatory cytokine INF-gamma and decreased levels of inflammatory cytokine TNF-alpha in plasma. These results identify a protective role of the BEP neuron against the metastatic diffusion possibly via increasing the innate immune function and reducing the inflammatory milieu.

### Example 5

### Prenatal Alcohol Exposures Reduces Progenitor Cells Differentiation to BEP Neurons while Nanosphere's Delivered cAMP Increases BEP Neuron Growth

It has been demonstrated that prenatal exposure to ethanol causes a significant decrease in the number of beta-endorphin (BEP) neurons in the hypothalamus in rats. Although ethanol is known to induce BEP neuronal apoptotic death, it is not known whether it also alters differentiation of BEP neurons from neuronal progenitor cells. Using an early postnatal binge ethanol administration model and bromdeoxyuridine (BrdU) labeling method, it is shown here that ethanol reduced the number of differentiated BEP neurons. Similarly, moderate dose of ethanol inhibited the differentiation and maturation of BEP neurons by cAMP-activating agents in primary cultures of fetal hypothalamic neuronal progenitor cells. Since the cAMP-activating agent increased BEP neuron differentiation in vitro, the effect of nanosphere-delivered cAMP into the 3rd ventricle on endogenous progenitor cell differentiation to BEP neurons in adult male rats was tested. It was found that nanosphere-delivered cAMP significantly increased the number of BEP neurons in these rats. Functional studies using NK cell response to lipopolysaccaride in the nanosphere-treated rats verified the histological data of BEP neuronal growth. These data indicate that prenatal ethanol exposure alters progenitor cell differentiation to reduce the number of BEP cells. Furthermore, the data provide first evidence to show the use of nanosphere's delivered cAMP to generate new BEP neurons to reduce immune and stress problems in fetal alcohol exposed subjects.

### Example 6

It has been observed profoundly that fetal alcohol exposure increase both basal corticosterone levels and corticosterone response to stress in rodent models (Sarkar et al. (2007) Endocrinology, 148:2828-34). However, it is not known whether this hyper-activity of stress axis can be transmitted to the offspring, and how are the abnormalities transmitted. Therefore an experiment was conducted by breeding fetal alcohol exposed Sprague Dawley rats with control animals of the opposite gender. In this way, two different germlines were produced—male germline by breeding male fetal alcohol rats and their male offspring with normal females (AFM), and female germline by breeding female fetal alcohol rats and their offspring with normal males (AFF). F1˜F3 generations were sacrificed at 2 months old by decapitation. Half of the animals were treated with i.p. injection of LPS (100 μg/kg body weight, Sigma) and sacrificed after 2 hours. Trunk blood plasma was collected, and plasma corticosterone levels were analyzed by ELISA (Diagnostic System Laboratories, Webster, Tex.). F2 and F3 generation offspring of fetal alcohol exposed animals continue to show increased basal levels of corticosterone and increased corticosterone response to LPS, which is considered as an immune challenge. This indicates that hyper-activity in HPA axis can be transmitted through male germline to at least the third generation after fetal alcohol exposure.

### Example 7

Fetal alcohol exposed animals have diminished immune function such as decrease of NK activity (Arjona et al. (2006) Alcohol Clin. Exp. Res., 30:1039-44). Research using animal models has shown that prenatal ethanol exposure negatively affects lymphoid tissue development, immune cell function, humoral immunity, and cytokine secretion (Zhang et al. (2005) Exp. Biol. Med., 230:376-88). NK cells are part of innate immune system, which form the first line defense against viral infection, tumor growth and metastasis. NK cells are sensitive to stress-induced hormones, which down-regulate NK cytolytic activity. Therefore it was hypothesized that NK cell function is also deceased in fetal alcohol animals. Here it was found that adult rats exposed to ethanol during their fetal life had reduced expression of granzyme B and interferon γ (IFN-γ) together with decreased NK cell cytotoxic activity (Arjona et al. (2006) Alcohol Clin. Exp. Res., 30:1039-44).

Since decreased number of β-EP neurons was observed in fetal alcohol exposed animals, and β-EP negatively regulate the activity of stress axis hormones such as CRH and corticosterone, which function as immune suppressors, it was hypothesized that the immune dysfunction in fetal alcohol animals is primarily caused by decreased production of β-EP peptide in central nervous system (CNS), and that this immune dysfunction can be reversed by replenishing the β-EP population in the hypothalamus.

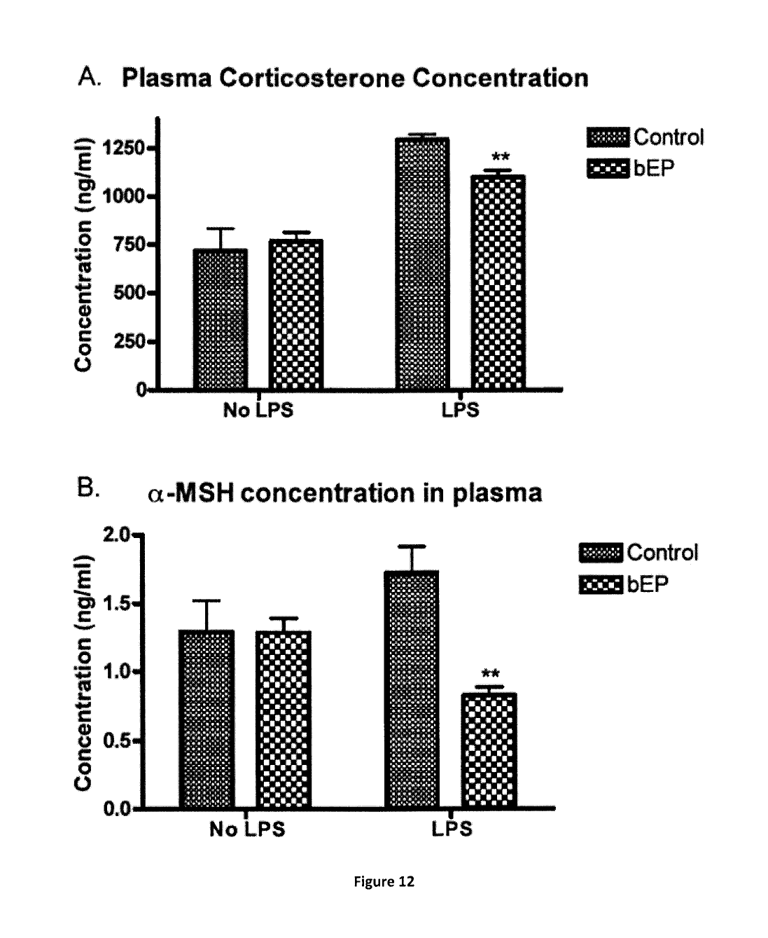

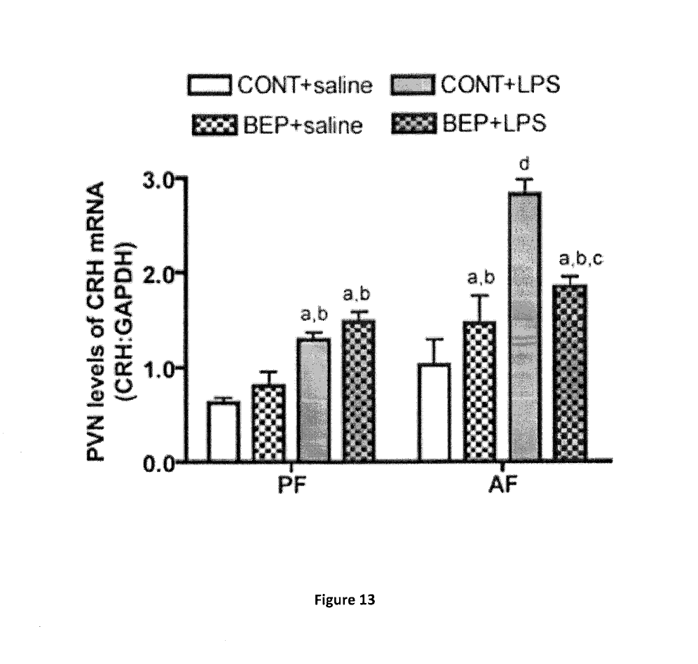

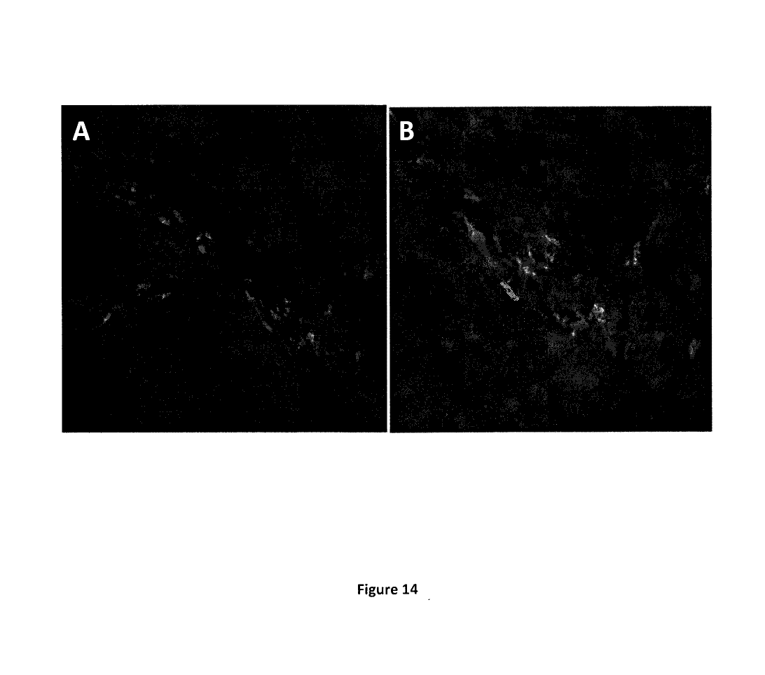

To increase the population of β-EP neurons in the hypothalamus, one possible method is to transplant in vitro cultured β-EP neurons into the brain. A method was developed to culture and enrich hypothalamic neuronal stem cells in vitro and differentiate them into neurons that express β-EP peptide. After these in vitro-differentiated cells were transplanted into PVN (paraventricular nucleus) of hypothalamus, where the CRH-producing neurons reside, these cells could integrate into the surrounding environment and down-regulate the activity of stress axis under immune challenge with LPS in both normal and fetal alcohol exposed animals (FIGS. 12 and 13), which indicates a role of decreased β-EP neurons played in the hyper-activity of stress axis in fetal alcohol animals.

One possible concern with the effect of β-EP transplantation therapy is that whether the transplanted neurons are going to survive and function normally until the time when the physiological status (such as immune functions and resistance to cancers) of these animals is examined. It has been shown above that BrdU-labeled neurons remained at the site of transplantation after 2 weeks. β-EP immunohistochemistry staining shows that the cluster of β-EP neurons remained in the PVN area at least after 13 weeks of transplantation surgery in Sprague Dawley females (FIG. 14A, 14B).

### Example 8

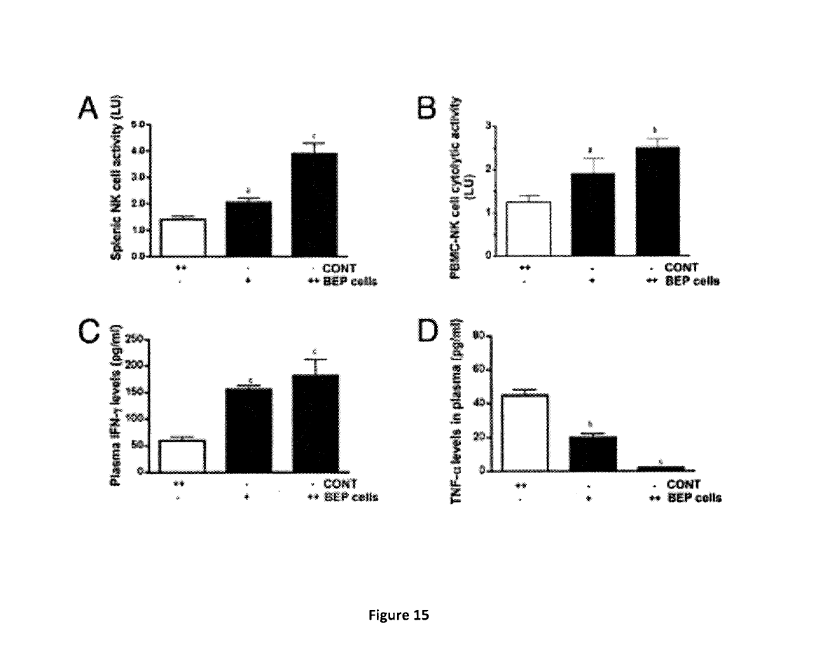

Central β-EP secretion is believed to reduce corticotrophin releasing hormone (CRH) neuronal mediate sympathetic influence on NK cell activity in the spleen (Boyadjieva et al. (2006) Alcohol Clin. Exp. Res., 30:1761-7). Work using adult Sprague Dawley rats showed that transplantation of β-EP neurons into PVN of hypothalamus increases NK cells cytolytic activity and plasma level of interferon-γ (IFN-γ), which triggers the protective defenses of the immune system that eradicate pathogens or tumors, and decreases plasma level of tumor necrosis factor-α (TNF-α), which induces inflammation reaction (FIG. 15). However, the long-term effect of β-EP transplantation on immune function was not evaluated. Here male Fischer animals were used, which received transplantation of β-EP neurons at the age of 50˜60 days old, and took their blood samples after 6˜8 months of the transplantation to check their blood hormone level and activity of peripheral blood mononuclear cells (PBMCs). As is shown above, β-EP animals still showed inhibitory effect of stress axis after LPS stimulation. β-EP animals also reversed the increase of α-MSH level after immune challenge.

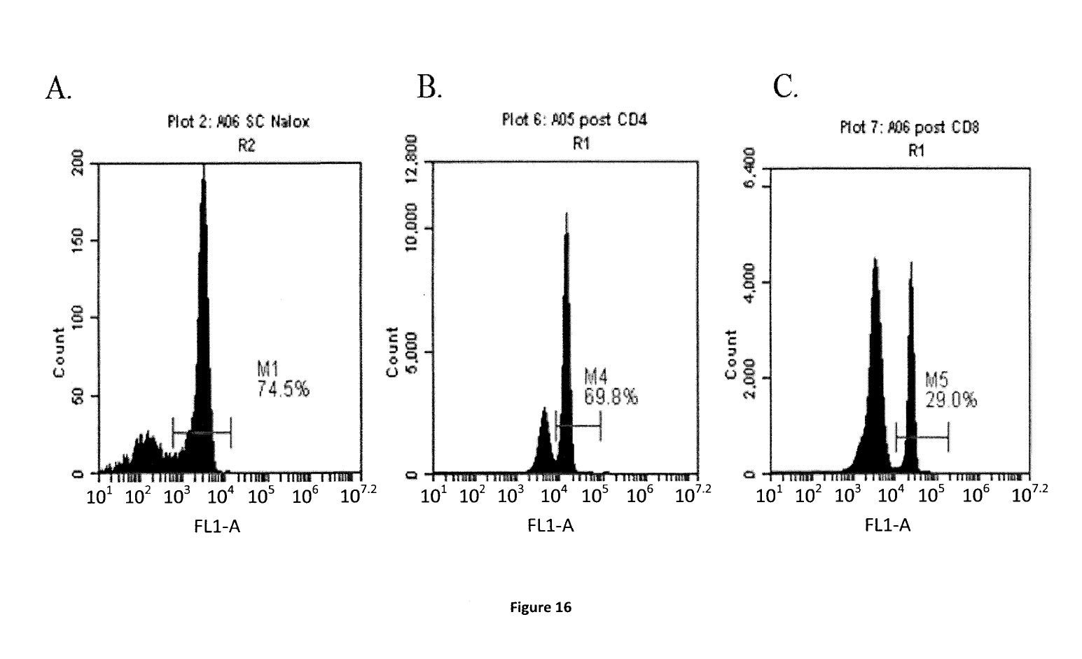

A method to enrich natural killer cells for cytotoxicity assay from orbital puncture blood has also been developed using automatic magnetic-activated cell sorting (MACS) machine. FITC-anti-granulocyte and MHC-II antibodies were used as primary antibodies, then anti-FITC, CD45RA and pan T antibodies which are linked to magnetic beads were used as secondary antibodies. Then cells were passed through MACS machine, in which all the labeled cells were eliminated. By using this method, intact NK cell could be collected with around 70% purity (FIG. 16A). Similarly, by substituting pan T antibody with anti-CD161a antibody, intact T cells population could be enriched to close to 100% (70% CD4+ and 30% CD8+) purity (FIG. 16B, C).

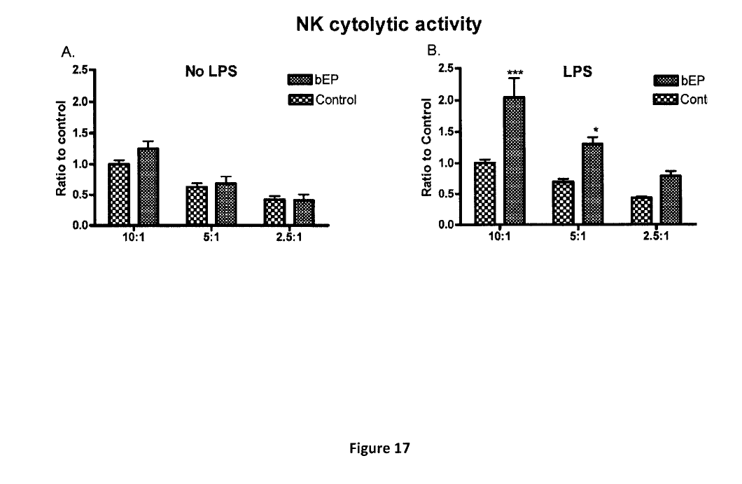

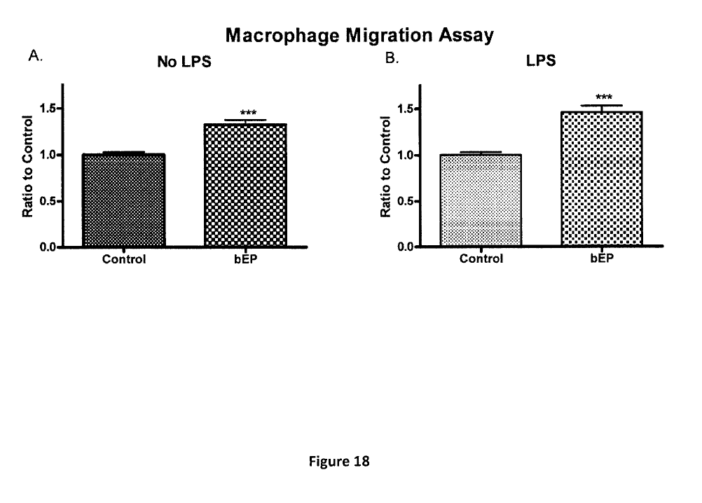

By using this developed cell-purification technique, live NK cells from orbital puncture blood of Fischer rats were enriched which have been transplanted with β-EP cells 6˜8 months ago, and co-cultured them with MADB106 cells, which is an NK-sensitive breast cancer cell line. After 3 hours, cytolytic activity was measured using CytoTox-ONE homogeneous membrane integrity assay (Promega). Here it was shown that NK cytolytic activity from β-EP transplanted animals is significantly higher than control animal under LPS stimulation (FIG. 17) even after 6 months of transplantation. Not only NK cells, blood monocytes in these animals also showed higher migration activity both with and without LPS stimulation (FIG. 18).

### Example 9

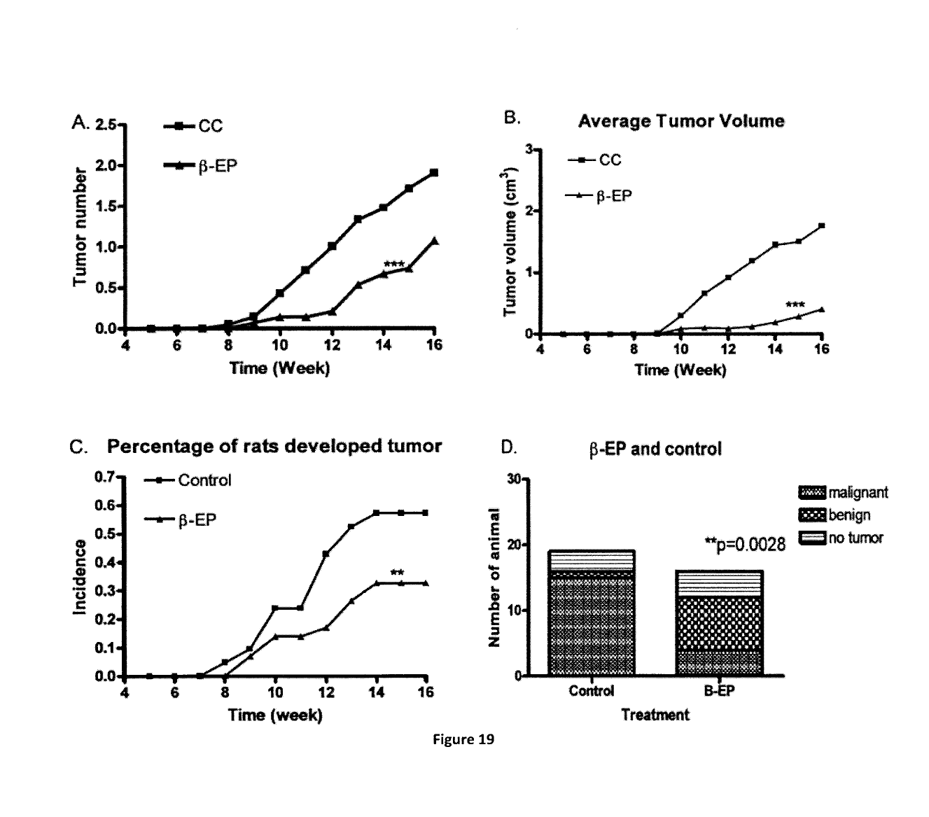

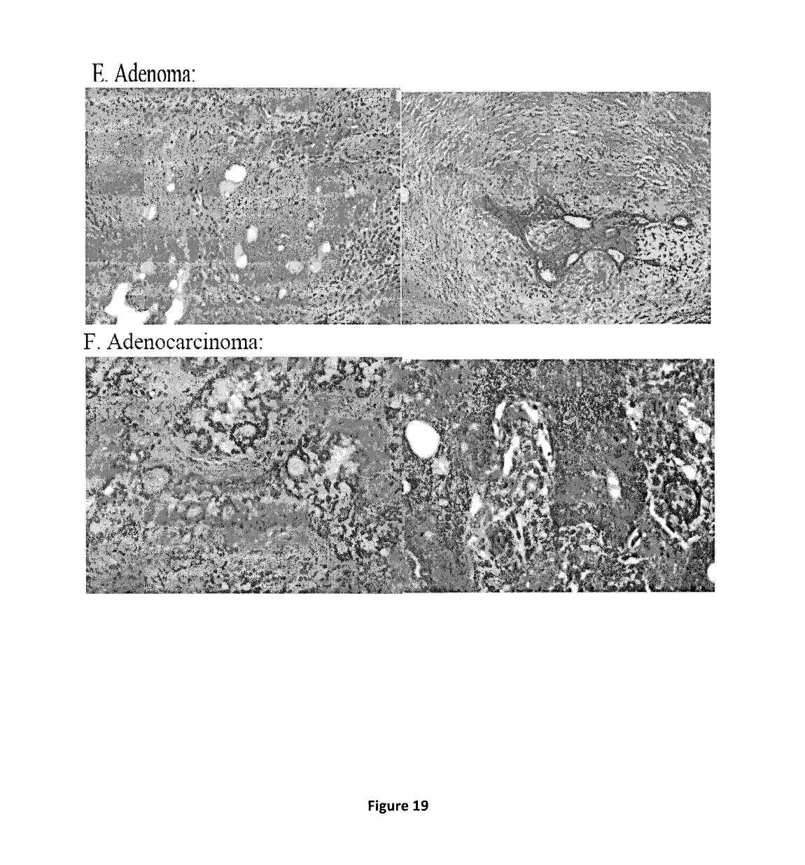

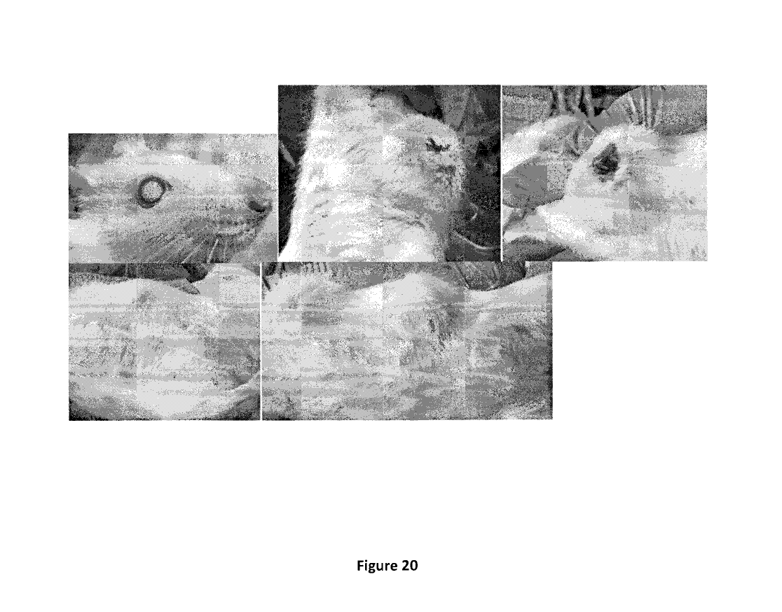

It has been shown that β-EP neuron transplantation could reduce incidence of chemical-induced prostate cancer. Since it has been shown that β-EP neuron transplantation could decrease activity of stress axis and increase immune function, it was hypothesized that β-EP neuron transplantation can also suppress development of other types of chemical-induced cancer, such as MNU-induced breast cancer. 50 days old virgin Sprague Dawley rats were injected with a dose of MNU (50 mg/kg body weight). After 1 month of NMU injection, animals were anesthetized and injected with cortical cells (CC, as control) or β-EP neurons (β-EP) in both sides of PVN of brain hypothalamus using stereotactic device. No tumor was detected at this time. 1 week after the brain surgery (5 weeks after NMU injection), animals were palpated every week to check for tumor growth. Tumor length and width were measured with a calibrator. 16 weeks after NUM injection, animals were sacrificed, tumors were collected, and slices of tumors were put in formalin and processed for histology staining. It was found that β-EP implanted animals have significantly lower tumor incidence, tumor volume, tumor number and malignancy rate (FIGS. 19 and 20).

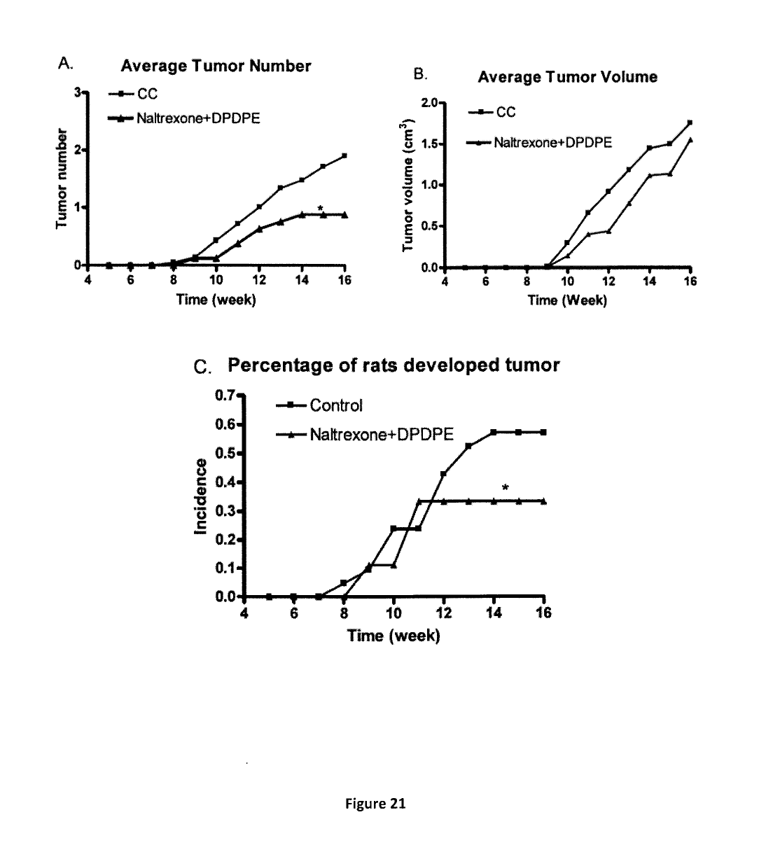

It has been shown that chronic administration of naltrexone suppresses μ-opiate receptor binding but increases δ-opiate receptor activity in rat splenocytes, thus enhances NK cell cytolytic activity response to BEP in vitro. Chronic naltrexone treatment increases NK cell cytolytic activity and cytokine production in the spleen in vivo (Boyadjieva et al. (2004) J. Immunol., 173:42-9). It was hypothesized that by using chronic naltrexone treatment, and a δ-opioid receptor agonist, DPDPE ([pC1-Phe4]-DPDPE), a series of opioid-like effects could be activated on immune function similar to high dose of β-EP treatment, and inhibit MNU-induced breast cancer growth. 9 weeks after NMU injection, 9 animals were implanted with naltrexone pellets (100 mg, 60 days release) under skin. The other animals were implanted with placebo pellets. 7 days after naltrexone pellet implantation, DPDPE (100 μg/kg body weight) was injected by i.p. every day until animals were sacrificed. It was shown that animals treated with naltrexone+DPDPE had decreased number and incidence of breast tumors (FIG. 21).

### Example 10

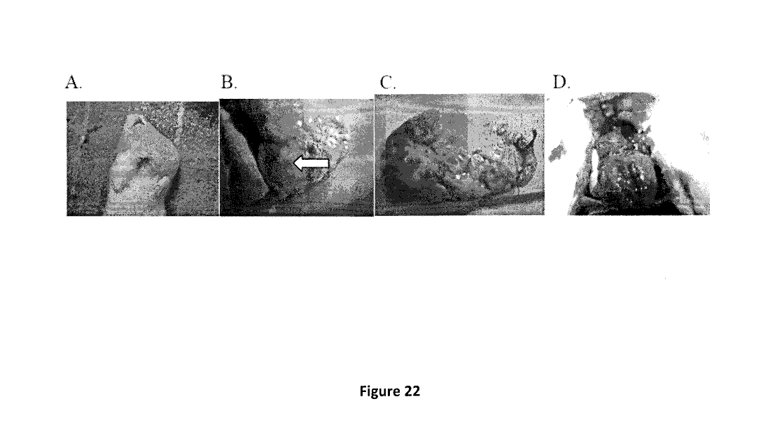

A method for lung metastasis was adapted using MADB106 breast cancer cells, a NK-sensitive cell line widely used for lung metastasis study. Animals were anesthetized, and 100,000/0.2 ml/rat of MADB106 cells suspended in media were inoculated into jugular vein. After 4˜6 weeks, 70˜80% of inoculated animals developed tumor at the position of lung and/or neck, where the tumor cells were inoculated into the jugular vein. Following shows different severities of tumors developed in animals (FIG. 22).

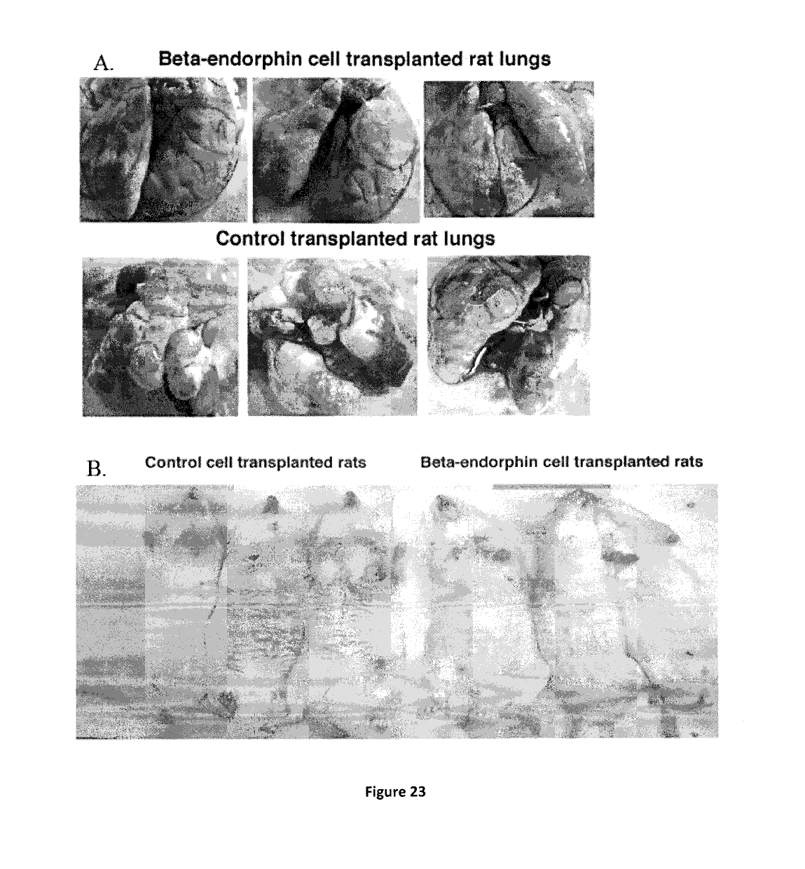

β-EP transplantation showed efficiency in clearing up MADB106 cells from the blood. A single case of tumor in β-EP transplanted animals has not been observed so far, which means, β-EP transplantation completely eliminates retention of MADB106 tumor cells in the lung (FIG. 23).

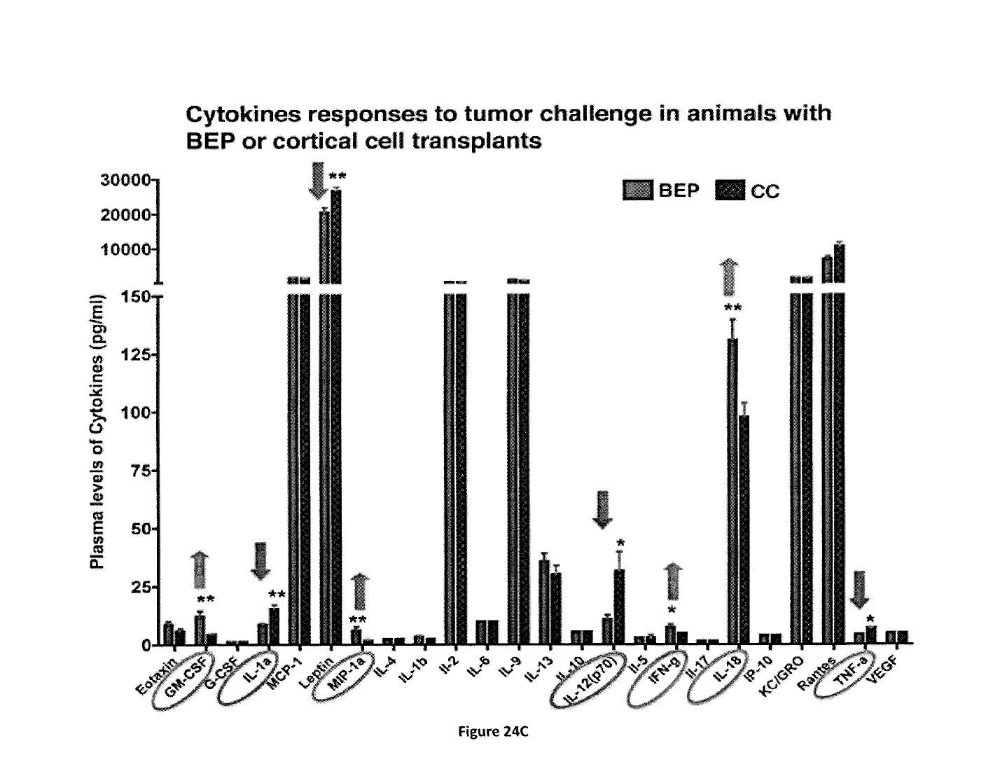

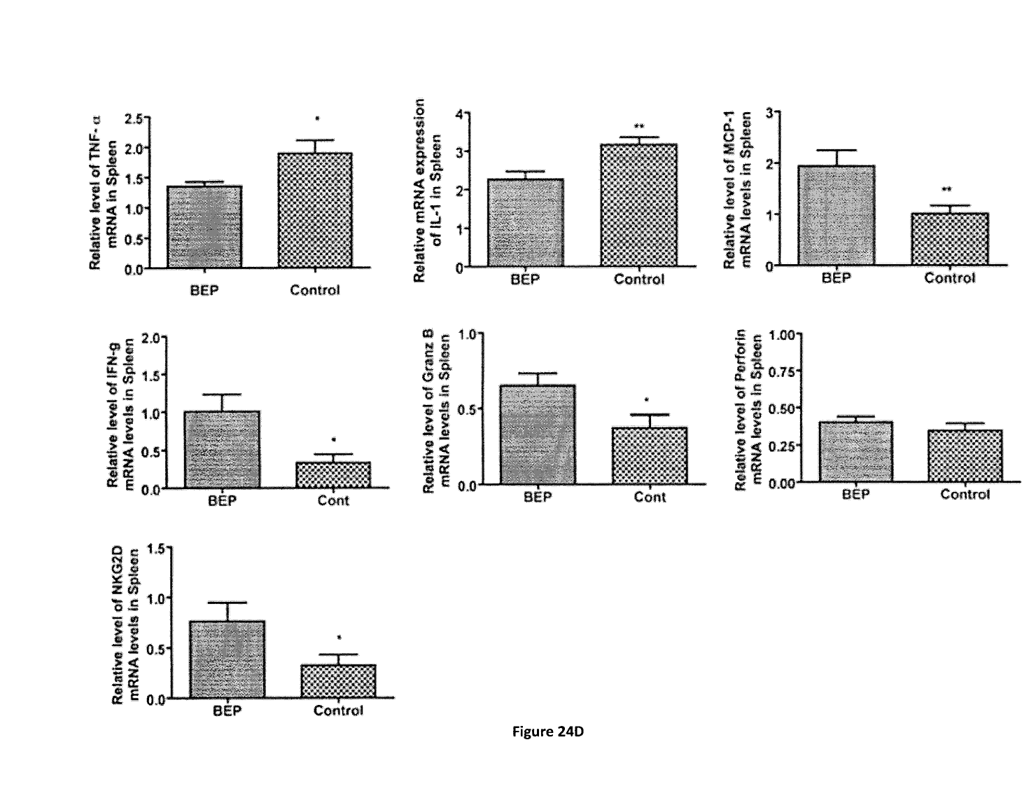

Since β-EP transplantation could increase immune activity in normal animals, it was hypothesized that the removal of tumor cells from the lung of β-EP transplanted animals is also cause by increased immune function. The immune reaction to tumor cell inoculation in male Fischer animals was checked after they received β-EP transplantation for 8˜9 months. Animals were anesthetized with Nembutal, 1 ml of blood was drawn from jugular vein, and then MADB106 cells (100,000/0.2 ml/rat) were inoculated into jugular vein. After 24 hours, animals were anesthetized again. 1 ml of blood was drawn from jugular vein, and then animals were sacrificed by decapitation. Vein blood before and after tumor inoculation was used for flow cytometry to determine cell populations in PBMC. Trunk blood PBMCs and splenocytes were used for migration assay. Trunk plasma was used for multi-cytokine assay. Part of the spleens was used for quantification of mRNA expression. It was found that β-EP animals have higher macrophage/monocyte migration activity in both PBMCs and splenocytes (FIG. 24A), higher NK cell percentage in PBMCs (FIG. 24B), higher GM-CSF, MIP-1α, IL-18 and IFN-γ, and lower IL-1α, IL-12 and TNF-α, in plasma (FIG. 24C) after tumor stimulation comparing to control animals. Similar to cytokines detected in plasma, examination of mRNA expression in spleen showed that β-EP animals had higher MCP-1, INF-γ, Granzyme B and NKG2D, and lower TNF-α and IL-1 expression after tumor cell inoculation comparing to control animals (FIG. 24D).

### Example 11

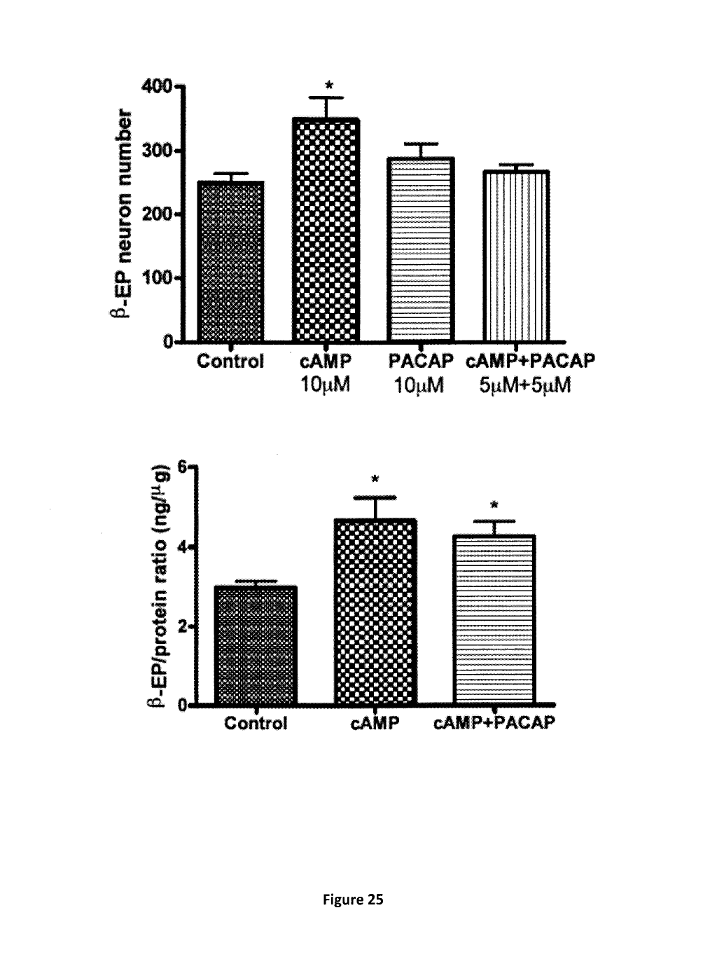

A method to induce differentiation of NSCs in the host itself using the same molecule as used to differentiate neuronal stem cell into β-EP in culture—cAMP and PACAP—was also evaluated. Since neuronal differentiation from NSC in the neuroepithelium of the third ventricle persists in the adult, which is part of the neuronal plasticity, it is possible to induce NSC to differentiate into β-EP neurons in vivo by administering cAMP-activating agents into the third ventricle, where NSCs are located. To immobilize and ensure a slow release of cAMP molecules, a “nanosphere” was used as a carrier. Nanospheres are commercially made spherical particles, typically 13-20 nanometers (nm) in diameter. Each nanosphere can be functionalized with a defined number of certain molecules. When injected into the third ventricle where neuronal stem cells reside in the epithelium, these nanospheres settle down and act as signaling to cell differentiation. It has been proven that β-EP neurons can be differentiated in situ from neural stem cells by nanosphere-delivered-cAMP into the third ventricle (FIG. 25). Besides increased β-EP neuronal numbers in cAMP-containing nanosphere implanted animals, it has also been found increased β-EP peptide level in hypothalamus of both cAMP and cAMP+PACAP-containing nanosphere implanted animals (FIG. 25).

Nanospere-delivered cAMP significantly increased the number of β-EP neurons (FIG. 25) as well as elevated the hypothalamic levels of β-EP peptide (FIG. 25). Nanosphere-delivered PACAP had a very moderate or no effect on cell differentiation. These data indicate that β-EP neurons can be differentiated in situ from NSCs by nanosphere-delivered-cAMP into the third ventricle.

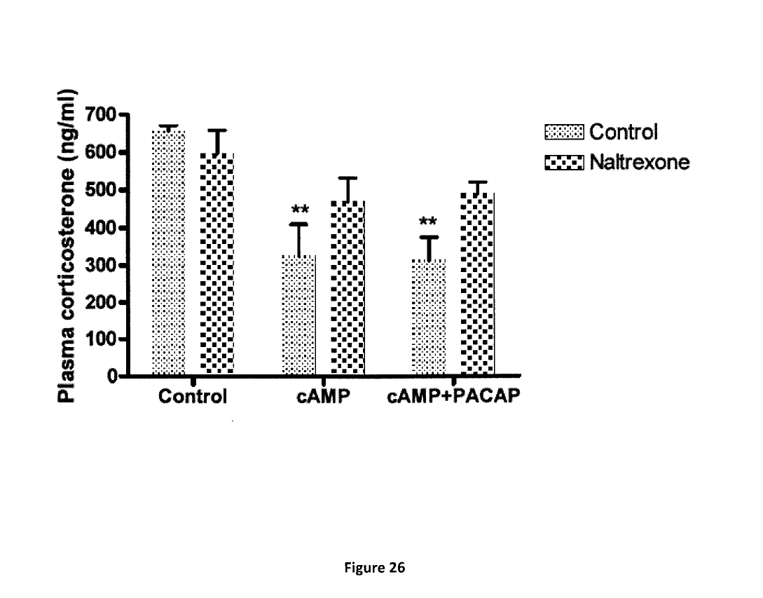

Since cAMP-delivering nanosphere implantation could increase β-EP neuron number and peptide production in the hypothalamus, and increased β-EP neuron number could decrease the activity of stress axis, it was hypothesized that cAMP-delivering nanosphere implantation may be able to decrease stress axis activity in rats. Here it is shown that both cAMP and cAMP+PACAP nanospheres decreased plasma corticosterone level, and that the inhibitory effect of nanospheres was reversed by naloxone, a μ-opioid receptor antagonist (FIG. 26).

Since β-EP neurons are known to play an important role in regulating the body stress control by inhibiting the function of the hypothalamic-pituitary-axis and plasma level of corticosterone, it was tested whether cAMP-delivering nanosphere implantation can decrease plasma levels of corticosterone in rats. Furthermore, in order to verify that the changes in corticosterone levels is brought in by elevated levels of β-EP, it was determined effects on corticosterone levels after blocking the activity of the opiod peptide by a blocker naloxone. Here it is shown that implantation of nanospheres containing cAMP or cAMP+PACAP decreased plasma corticosterone level, and that the inhibitory effect of nanospheres was reversed by naloxone, a μ-opioid receptor antagonist (FIG. 26).

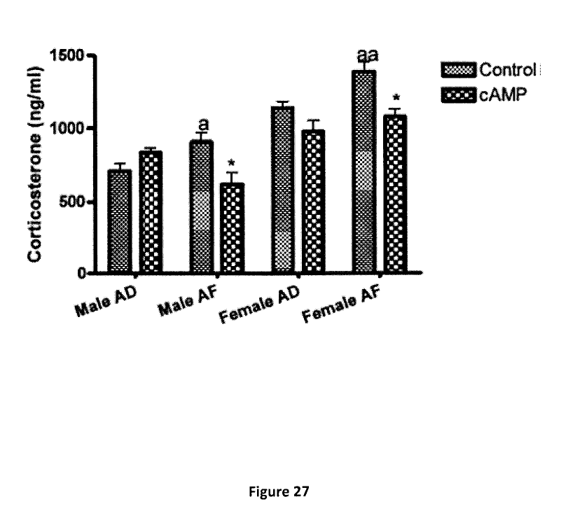

It is already known that fetal alcohol exposure increases basal plasma corticosterone level in adult animals. It has been shown that cAMP-containing nanospheres implanted into 3rd ventricle could decrease corticosterone in normal female Fischer rats. Here it was determined whether the nanosphere implantation can also decrease corticosterone level increased by fetal alcohol exposure. As is shown in FIG. 27, cAMP-delivering nanosphere implantation significantly decreased corticosterone levels in both male and female fetal alcohol exposed animals.

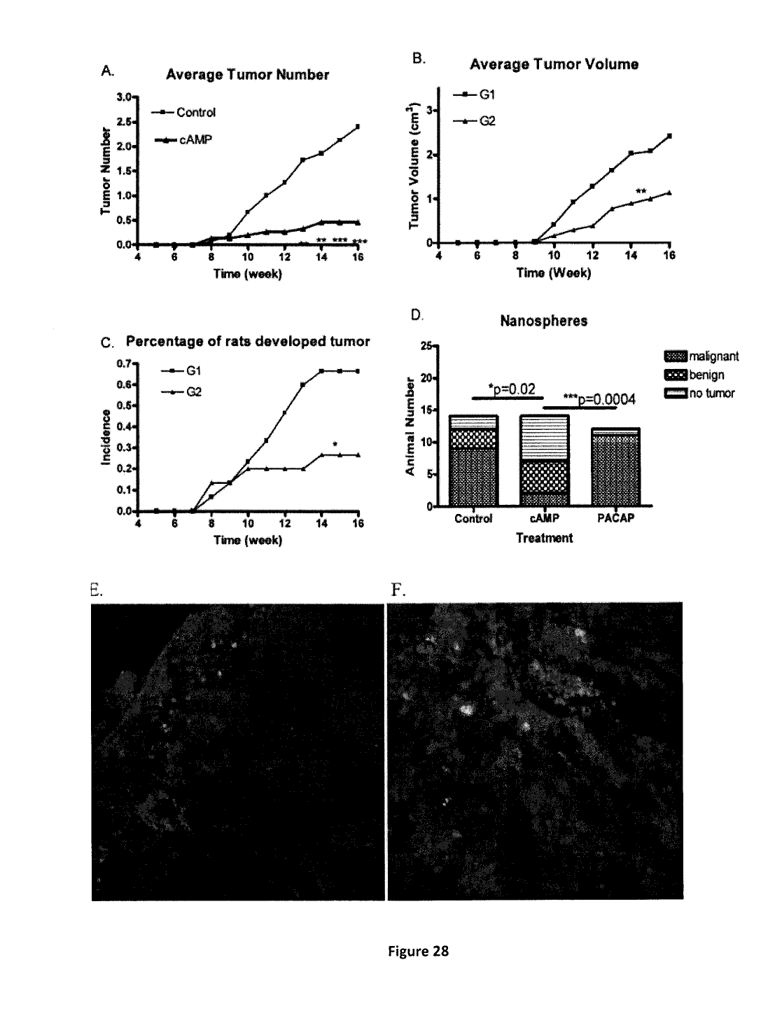

Since it was also found that cAMP-delivering nanospheres increase β-EP neuron numbers in the hypothalamus, it was hypothesized that nanospheres implanted into the 3rd ventricle could also reduce incidence and development of MNU-induced breast cancer. Using similar procedure as described above, 1 month after MNU injection, two different kinds of nanospheres were implanted—control (Plain Melamine Fluorescent Nile Blue nanospheres) and cAMP (Melamine Fluorescent Nile blue nanospheres that deliver 70 nmole of cAMP in ˜10 μl). Tumor size was monitored as described above. It was found that cAMP-containing nanosphere implanted into 3rd ventricle significantly decreased tumor development in MNU-induced breast cancer (FIG. 28).

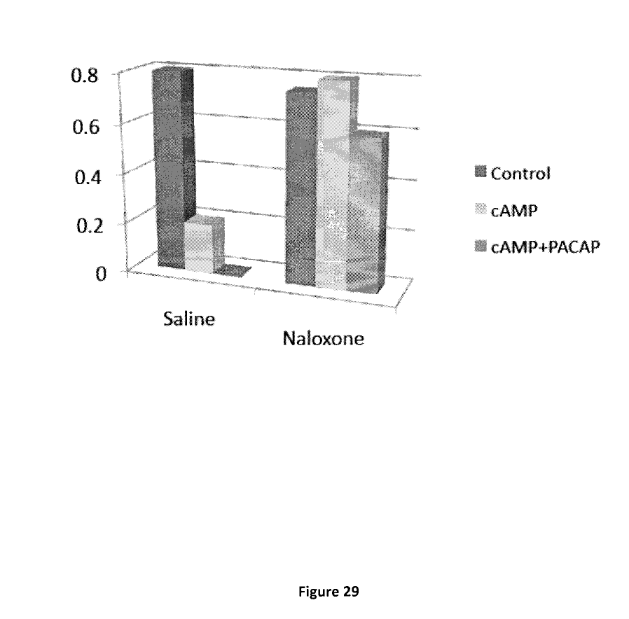

Since it has been shown that cAMP-delivering nanospheres implanted into 3rd ventricle increase β-EP neuron number in the hypothalamus, and decrease tumor development in breast cancer, one could expect that implantation of these nanospheres also inhibits tumor development of MADB106 lung metastasis. 30 female Fischer rats were implanted with control, cAMP (10 μM) or cAMP (5 μM)+PACAP (5 μM) nanospheres into 3rd ventricle. 2 weeks later, half of the animals were randomly assigned to treatment with Naloxone (1 mg/kg body every 12 hours) for 3 times. After the second injection of Naloxone, animals were anesthetized and inoculated with 100,000/0.2 ml/rat MADB106 cells. 2 months later, animals were sacrificed, and lungs were fixed and sectioned for histology diagnosis. It was found that cAMP and cAMP+PACAP nanospheres both decreased the rate of lung tumor retention, and naloxone, which is a μ-opioid receptor antagonist, reversed the effect of nanosphere (FIG. 29).

These data indicate that slow delivery of cAMP activating agents via nanospheres into the 3rd ventricle increases the number and activity of endogenous β-EP neurons, and resulting in suppression of stress response, reduction of MNU-induced mammary tumor growth and progression and prevention of lung metastasis of mammary cancer cells. Furthermore, these data identify a novel therapeutic approach to prevent stress and immune diseases and cancer.

### Example 12

It has been shown that β-EP transplantation increases POMC gene expression and β-EP peptide production in the central nervous system, but not in peripheral blood. This fact indicates that the effect of β-EP on immune system such as NK cell activity is transmitted by some intermediary factors. It has already been shown that β-EP negatively modulates activity of HPA axis, and down-regulates production of CRH and corticosterone, which are potent inhibitors of immune activity. However, the inhibitory effect on stress axis of β-EP may not completely explain the elevated immune function and the efficiency of tumor clearance. Since immune organs such as spleen is highly innervated with sympathetic and parasympathetic nerves, and norepinephrine released from sympathetic nerves as neurotransmitter inhibits NK cell activity, it is hypothesized that the beneficial effect of β-EP on immune function is also regulated through autonomic nervous system (ANS).

It was hypothesized that central β-EP modulate peripheral immune function through inhibiting sympathetic nerves and activating parasympathetic nerves. Metaproterenol is an agonist for β-receptors, which are a class of G protein-coupled receptors that are targets of the catecholamines, especially norepinephrine and epinephrine. Methyllycaconitine is a selective antagonist of α7 nicotine acetylcholine receptor (α7 nAChR), which is triggered by neurotransmitter acetylcholine from parasympathetic nerves. By i.p. injecting metaproterenol and methyllycaconitine into whole body, the stimulation of sympathetic nerves and inhibition of parasympathetic nerves was mimicked. According to the hypothesis, one would expect that both metaproterenol and methyllycaconitine will inhibit the beneficial effect of β-EP transplantation.

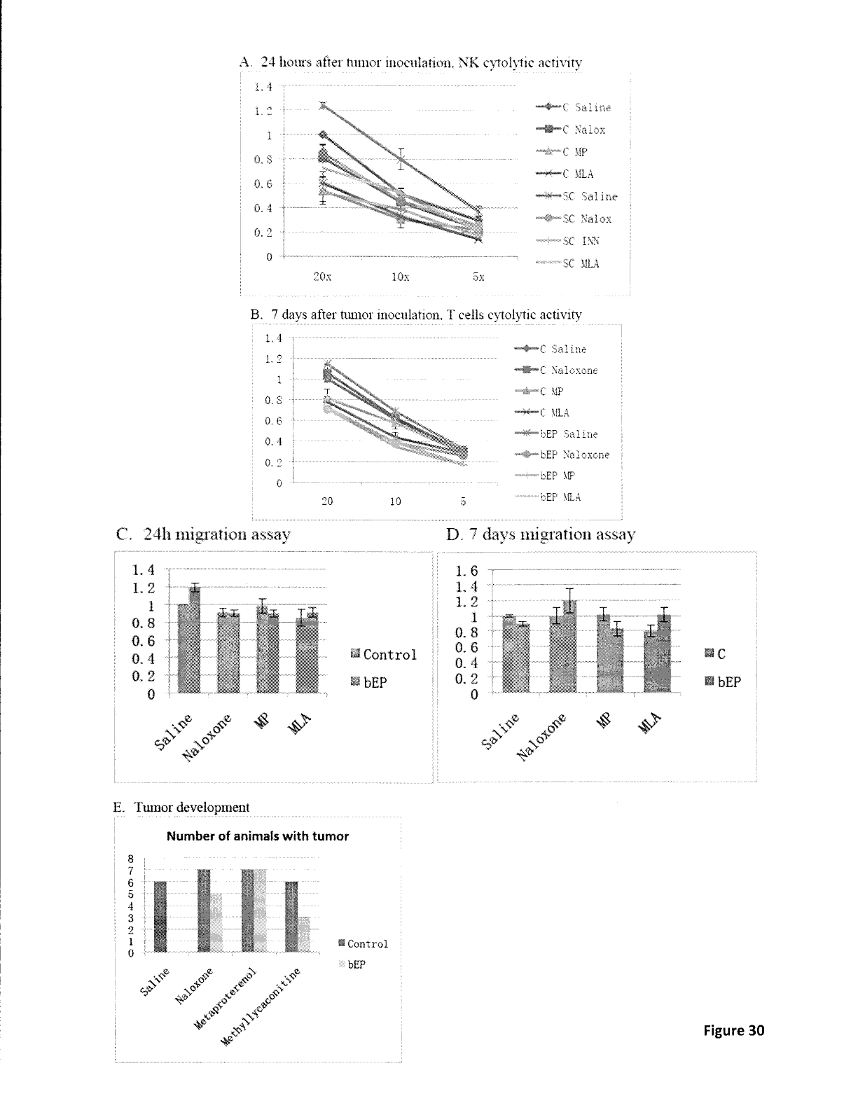

48 β-EP implanted (SC) male Fischer rats and 48 control (C) male Fischer rats were divided into 8 groups, treated with saline, Naloxone (10 mg/kg body weight), Metaproterenol (MP, 0.8 mg/kg body weight), or Methyllycaconitine (MLA, 2.5 mg/kg body weight), respectively. Animals were treated with drugs for 8 days. In the second day of drug injection, animals were inoculated with MADB106 cells (100,000/0.2 ml/rat). 24 hours after tumor cell inoculation, blood was collected by orbital puncture. Plasma was collected, and PBMCs were separated for migration assay and NK cytolytic assay. 7 days after tumor inoculation, blood was taken again by orbital puncture. Plasma was collected, and PBMCs were separated for migration assay and T cell cytolytic assay. After 6 weeks, animals were sacrificed, and brains, spleens and blood were frozen. Lungs were fixed in formalin and processed to H&E staining. It was found that 24 hours after tumor inoculation, NK cell activity (FIG. 30A) and macrophage migration activity (FIG. 30C) are higher in β-EP animals injected with saline. MP and MLA both reversed the effect of β-EP. However, 7 days after tumor inoculation, there isn't a clear pattern of T cell cytolytic activity and macrophage migration activity between groups (FIGS. 30B and 30D). Final tumor result (FIG. 30E) showed that all the treatments: naloxone, MP and MLA stopped the effect of β-EP to completely eliminate tumor cell lung retention, indicating that all of these receptors—μ-opioid receptor, β-receptor and nAChR—are involved in the effect of β-EP on immune function against cancer.

As is shown previously, activation of β-receptor and inhibition of nicotine acetylcholine receptor could decrease immune function and increase susceptibility to cancer. In contrary, it is also possible that inhibition of β-receptor and activation of nicotine acetylcholine receptor could increase immune function and inhibit cancer development.

### Example 13

In response to stressful stimuli, cascades of hormones are released following activation of the hypothalamic-pituitary-adrenal (HPA) axis. The hypothalamus releases corticotropin-releasing hormone (CRH) in turn stimulating proopiomelanocortin (POMC) gene expression, which is cleaved into biologically active subunits, including beta-endorphin (β-EP). In rodents, behavioral responses to stress may be mediated by β-EP inhibition of CRH potentially regulating allostasis. Indeed, upon stimulation by stressful stimuli, β-EP synthesis, primarily in the ventromedial arcuate nucleus of the hypothalamus, is activated by CRH release from the paraventricular nucleus (PVN), subsequently modulating the response by inhibiting CRH secretion (e.g., Poplawski et al. (2005) Alcohol Clin. Exp. Res., 29:648-655; Plotsky et al. (1991) Endocrinology 128:2520-2525). Additionally, endogenous opioid systems interact extensively with serotonergic and dopaminergic neurotransmission mechanisms in brain areas associated with anxiety behaviors, such as the amygdala (e.g., Zarrindast et al. (2008) Life Sci., 82:1175-1181; Zhang et al. (1996) Zhongguo Yao Li Xue Bao 17:314-317), suggesting another possible mechanism of β-EP action to regulate the provocation of anxiety behavior. It has been previously shown β-EP neuron transplantation into the PVN reduces CRH neuronal response in the hypothalamus in response to an immune stressor (Boyadjieva et al. (2009) Alcoholism: Clin. Exper. Res., 33:931-937). To determine if β-EP plays a role in mediating anxiety-like behavior, rats underwent β-EP neuron transplantation into the PVN and tested in two behavioral paradigms. In addition, it was determined corticosterone response to restraint stress in these rats compared to controls.

### Materials & Methods

### Preparation of NSC-βEP Cells:

From 17-days old pregnant rats (Simonsen Laboratories; Gilroy, Mass.), fetal rats were obtained to extract mediobasal hypothalamic tissues. Hypothalamic neurons were enriched by filtering and maintained in HDMEM (10% FBS) for a duration of 3 wks. At the 3rd week, stem-cell spheres were present. Spheres were separated and dissociated into single cells. Neurospheres were maintained in culture for several months. Stem-cell differentiation was performed by treating cells for 1 wk with PACAP and dbcAMP, or combination. Following the differentiation process, all cells were β-EP positive. Cortical cells were prepared as described above.

### Animal Surgery & Transplantation:

Male Fischer rats (n=40; Charles River) were housed individually under a standard 12:12 light-dark (LD) cycle in temperature controlled environment (˜22° C.) throughout the duration of the study. At ˜6 weeks of age, animals were anesthetized with sodium pentobarbital (50-70 mg/kg, I.P.; Henry Schein), and injected with 1 mL of stem-cell suspension into both lobes of PVN. Using a 54 Hamilton syringe, the coordinates were as follows: 0.5 mm from midline, 1.8 mm behind bregma, 0.5 mm lateral from bregma, and 7.5 mm below cortex; (see protocol, Sarkar et al. (2008) Proc. Natl. Acad. Sci., 105:9105-9110).

### Anxiety-Like Behavioral Testing Paradigms:

At approximately 16-18 weeks of age (β-EP, n=20; CC, n=20), animals underwent behavioral testing in the elevated-plus maze (EPM) and open-field (OF) apparatuses. All animals were tested in both the EPM and OF separated by a period of 3 weeks. To avoid possible order effects of the testing procedures, animals from both treatment groups were assessed in either the EPM or OF first followed by the other. To habituate to the testing room, animals were transported and kept in the testing room for at least 1 hour prior to each testing session. Following 1 hour, animals were gently removed from home cage and placed into the behavioral testing apparatus. All testing occurred between 14-16:00 hours. The duration of test session was recorded overhead by a high-resolution camera for subsequent behavioral scoring.

### Pharmacological Validation

Male Fischer rats (n=20) were injected I.P. with either diazepam (2 g/kg; DZP), an anxiolytic, or yohimbine (1.5 g/kg; YOH), an anxiogenic, at least 30 minutes prior to testing in either the EPM or OF.

### EPM Testing Session

The EPM is constructed from black acrylic pieces sitting 60 cm from the floor consisting of four arms (2 open and 2 closed) measuring 50 cm (L) by 10 cm (W). The testing room was illuminated with standard overhead fluorescent lighting (˜700 lux at arm level). The animal was placed in the center of the EPM facing an open arm and allowed to freely explore the apparatus for 5 minutes.

### OF Testing Session

The OF arena is a square with four walls measuring 90 cm (L)×90 cm (W)×40 cm (H). The arena is constructed from black acrylic non-transparent pieces. The testing room was illuminated with modified fluorescent lighting (˜25 lux at floor level). The animal was placed in the center of the arena and allowed to freely explore the apparatus for 5 minutes.

### Restraint-Stress & Tail-Blood Collection:

Prior to restraint, tail-blood was collected for basal corticosterone levels between 16-18:00 hours. Animals were restrained in Plexiglas tubing for approximately 60 minutes. During restraint, tail-blood was collected at 15 minutes intervals. Following restraint, blood was collected at 30 minutes intervals. All samples were kept on ice and immediately centrifuged to remove plasma for subsequent assays.

Animal surgery, care, and procedures were performed in accordance with institutional guidelines and complied with NIH policy. The animal protocol was approved by the Rutgers Animal Care and Facilities Committee.

### Corticosterone Assays:

Blood samples (˜80-100 μL) were collected in 154 of EDTA in 1.5 mL tube. Samples were centrifuged (4° C.; 3000 rpm) and plasma supernatant was drawn off. Samples were diluted 1:25 or 1:50 with provided buffer. Samples were run together in one assay plate to minimize variance of reagents and enzymes. Corticosterone levels were determined from 25 μL samples by Corticosterone ELISA kit (IBL, USA) per manufacturer's instructions. Standard curves were used to determine relative corticosterone levels.

### Statistics:

All treatment group results are means±SEM. Comparisons between groups were made using one-way ANOVA with Newman-Keuls Multiple Comparison post hoc test, two-ANOVA with Bonferroni post hoc tests, or Students t-test. Significance was set at α=0.05.

### Results

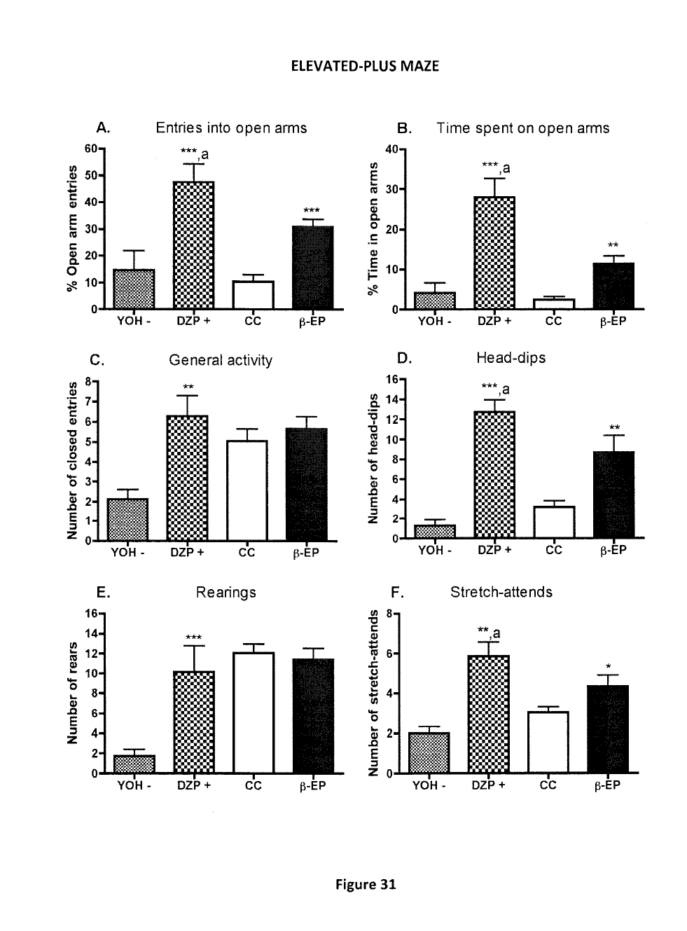

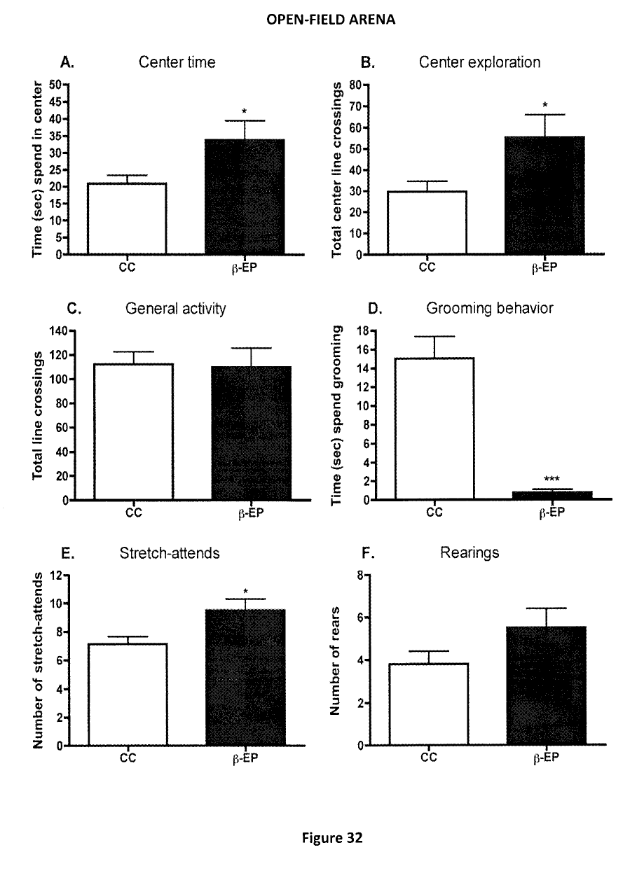

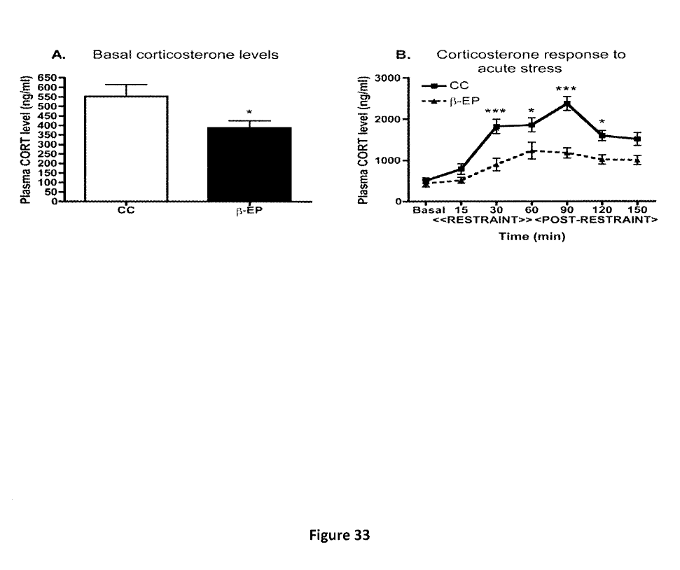

As seen in FIGS. 31-33, β-EP neuron transplantations into the PVN reduce the provocation of anxiety behavior in Fischer rats in two behavioral paradigms. The reduction in anxiety behavior is evident from standard measures in both paradigms, which are also reflected by changes in other behaviors associated with anxiety-like states. Corticosterone levels at baseline are reduced in β-EP neuron transplanted rats. During and following restraint stress, β-EP neuron transplanted rats displayed a significantly reduced corticosterone response. The data indicate β-EP regulates anxiety behaviors and modulates neuroendocrine changes reflecting responses to acute stress.

### Example 14

The aim of this study was to determine the efficacy of beta-endorphin (BEP) neuron transplants to prevent diethylnitrosamine (DEN)-induced rat hepatocelluar carcinoma. Male Fisher CDF rats were allocated to four groups. Rats implanted with cortical neurons: Group 1 (vehicle control) and Group 2 (DEN); and BEP neurons implanted group: Group 3 (vehicle control) and Group 4 (DEN). Rats implanted with cortical neurons (group 2) developed liver tumors with 60% tumor incidence; whereas, the BEP neurons implanted group (group 4) had no tumor during the 18-week study. The histopathological evaluation revealed that the liver of cortical neurons transplanted groups had various abnormalities such as extensive fibrosis, large focus of inflammatory infiltration and some animals had well differentiated HCCs, whereas, the BEP neurons transplanted group had no abnormalities. In addition, histological analyses for liver fibrosis and immunocytochemistry for placental glutathione S-transferase shows that, the cortical neurons transplanted group had severe extra cellular matrix deposition (ECM) and enhanced preneoplastic hepatic foci respectively, than the BEP neurons transplanted group. These observations suggest that BEP neuron transplantation prevents hepatocellular injury and hepatocellular carcinoma formation.

Hepatocellular carcinoma (HCC) commonly referred to as liver cancer is the most common form of liver cancer and is more frequent in men of ages 50-60. Hepatocellular carcinoma is caused by cirrhosis or scarring of liver tissue due to excessive alcohol consumption, including autoimmune diseases, Hepatitis B or C viral infection (Fattovich et al. (2004) Gastroenterology 127:S35-50). Surgery or liver transplant can treat only small or slow-growing tumors if detected early, yet most of the hepatocellular tumors remain undetected for timely treatment. Chemotherapy and radiation treatments are used to shrink the size of tumors which can be removed by surgery but with a higher risk of tumor recurrence (Fattovich et al. (2004) Gastroenterology 127:S35-50). Chemical hepatocarcinogenesis in rats is a useful experimental tool to study and develop cancer preventive interventions. In this study, a single treatment with an initiating liver carcinogen N-nitrosodiethylamine (DEN) and subsequent chronic administration of a tumor promoter 2-acetylaminofluorene (2-AAF) was used to develop liver tumors. DEN is a nitrosamine derivative. “Nitrosamines (R₂NN═O) are ubiquitous in biology and in the environment due to the abundance of nitrogen oxides and amine precursors, and they are generally considered to be carcinogenic” (Yi et al. (1995) J Amer Chem Soc., 117:7850-7851). Nitrosamines have been investigated for their interactions with heme-containing biomolecules and results in formation of carcinogenic metal complexes of nitrosamines like N-(hydroxyethyl)-protoporphyrin IX in livers of rats (Yi et al. (1995) J Amer Chem Soc., 117:7850-7851). 2-AAF is metabolically activated in cells and reacts with DNA to form primarily N-(deoxyguanosin-8-yl)-2-acetylaminofluorene (dG-C8-AAF) and N-(deoxyguanosin-8-yl)-2-aminofluorene (dG-C8-AF)” (Yasui et al. (2004) Biochemistry 43:15005-15013). 2-acetylaminofluorene and 2-aminifluorene induce base substitutions and frameshift mutations. 2-AAF can also induce site mutagenesis by promoting G>T transversions (Yasui et al. (2004) Biochemistry 43:15005-15013).

It has been shown that neural stem cell-derived BEP neurons transplanted into the hypothalamus suppressed carcinogen-induced prostate cancer development, mammary tumorigenesis and tumor metastasis. It was determined whether an analogous circumstance occurs in the liver. The focus of the study is to determine the effects of β-endorphin neurons implantation in rat hypothalamus on the development of diethylnitrosamine (DEN)-induced rat hepatocellular carcinoma.

### Materials and Methods

### Induction of Hepatocellular Carcinoma

Adult Fisher CDF rats (Charles River, Mass.) (180-210 g) were divided into 4 groups and fed water and basal diet ad libitum throughout the study and, were observed daily and weights were measured weekly. Cortical cell implanted groups: Rats were implanted with cortical cells into the paraventricular nucleus (PVN) of the hypothalamus and assigned to Group 1 (cortical cells vehicle control) then injected with 0.9% saline solution intraperitoneally and 0.5% carboxymethyl cellulose (CMC) intragastrically as a vehicle control. Hepatocarcinogenesis was induced in group 2 (cortical cells+carcinogen) rats by a single intraperitoneal injection of DEN (Sigma Chemical. Company, St Louis, Mo.) in 0.9% saline at 200 mg/kg body weight. Cancer was promoted by administering 2-AAF intragastrically, suspended in 0.5% CMC, beginning two weeks after DEN was injected. Promoter was administered for 3 days per week for 13 weeks. BEP cells implanted groups: Rats were implanted with in vitro produced BEP neurons into the paraventricular nucleus (PVN) of the hypothalamus and assigned to Group 3 (BEP cells vehicle control) and group 4 (BEP cells+carcinogen) rats. Group 3 animals were served as vehicle control as in group 1. Hepatocarcinogenesis was induced in group 4 rats by following the same protocol as for group 2. At the end of an 18 week period, all the animals were sacrificed by decapitation.

### Liver Histology

A portion of liver tissues were removed from sacrificed animals and fixed in 10% neutral buffered formalin for 24 hours. The fixed tissues were dehydrated in graded ethanol, embedded in paraffin, sectioned at 5 mm and collected on slides for H&E, Sirius Red, Massson's trichrome staining and immunohistochemical analysis. Fibrosis was semi quantitatively scored on Sirius red; Masson's trichrome staining sections as absent (0), mild (1), moderate (2) or severe (3).

### Immunohistochemical Localization of Placental Glutathione S-Transferase Hepatic Foci

Thin paraffin sections (5 μm) were mounted on Superfrost Plus glass slides (Fisher Scientific, Itasca, Ill.) and heated at 37° C. overnight. The sections were deparaffinized in xylene, gradually hydrated with decreasing concentrations of ethanol and water, and endogenous peroxidases were removed with 0.3% H₂O₂ in methanol for 30 minutes. Then the slides were subjected to antigen retrieval by microwave irradiation for 10 minutes at 98° C. in 0.1 M citrate buffer (pH 6.0). After cooling, the slides were incubated with 2% BSA in PBS at 25° C. for 30 minutes and subsequently incubated overnight at 4° C. with rabbit polyclonal antibody for placental glutathione S-transferase (MBL International, Woburn, Mass.). After the primary antibody incubation and PBS wash, sections were incubated with peroxidase-coupled anti-rabbit Ig ImmPRESS reagent (Vector Laboratories, Inc.). Antigen localization was achieved by using the 3,3′-diaminobenzidine-peroxidase reaction and sections were counter stained with Gill'#3 hematoxylin (1:4) as a blue nuclear counterstain, dehydrated, and cover slipped with Permount (Fisher Scientific). To evaluate the immunohistochemical staining, sections were photographed using a Nikon-TE 2000 inverted microscope. Intensity of staining was categorized as negative (−) and strongly positive (+++).

### Statistical Analysis

Statistical analysis was performed by using GraphPad Prism 5 software (GraphPad Software Inc., San Diego, Calif.). The chi-squared test was used to analyze the difference in tumor incidence at the termination of experiment. Differences were considered significant when the p value was <0.05.

### Results

### Tumor Incidence and Histopathological Observations

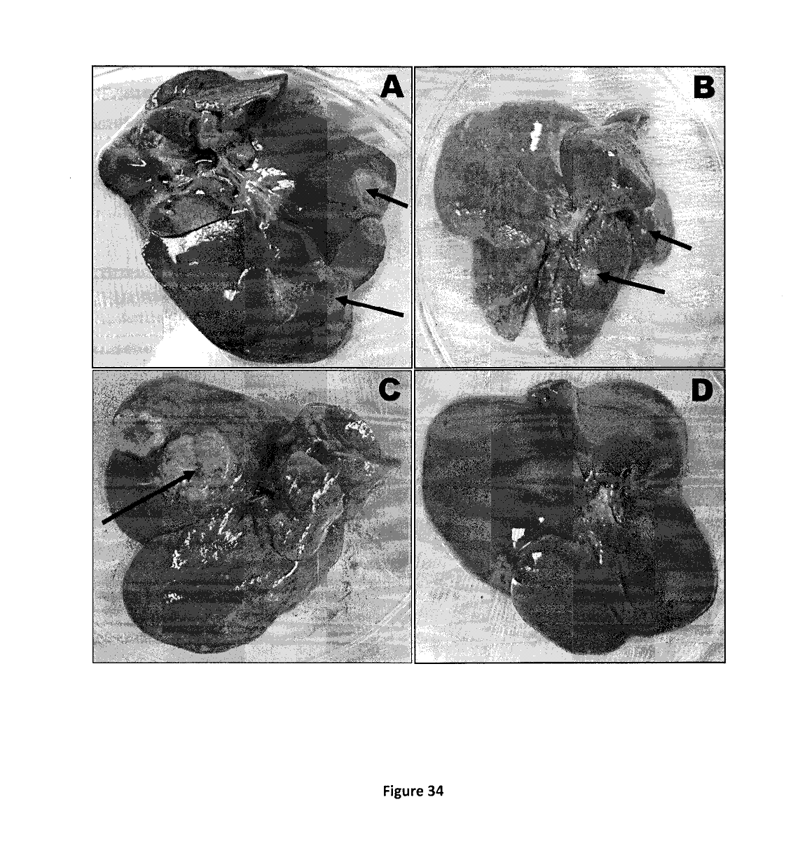

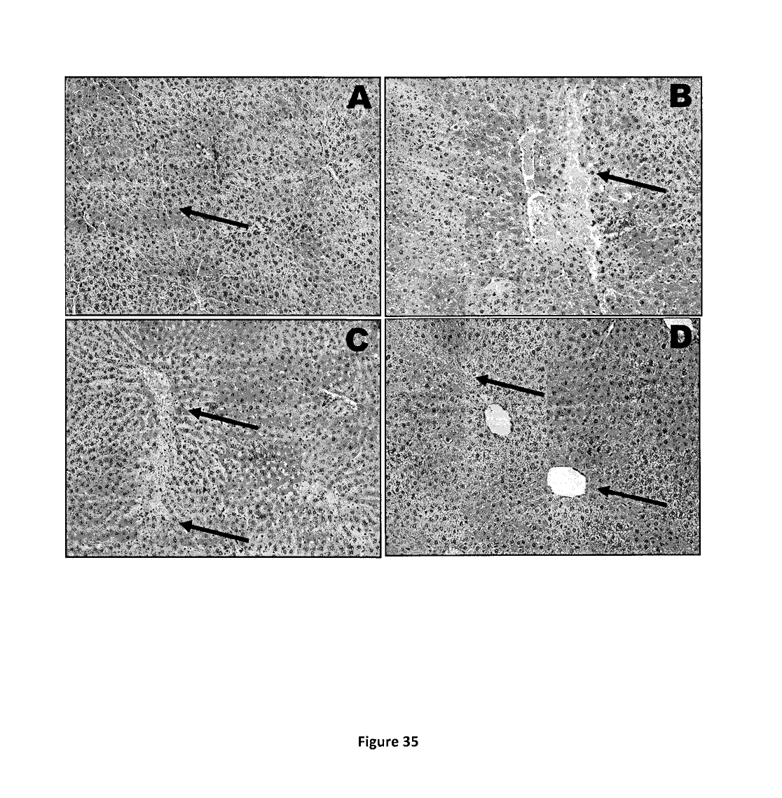

Interestingly, the BEP neuron transplanted group (group 4) had no macroscopic liver nodules or cirrhotic liver (FIG. 34D), in contrast cortical cells implanted groups (group 2) had tumor incidence of 60% (Table 2) and some of the animals showed cirrhotic liver (white mass) (FIG. 34A) and HCC tumor nodules (FIG. 34 B & C). No tumor was found in control groups 1 and 3 either. To explore whether BEP neuronal transplantation exerted beneficial effects at histological levels, paraffin-embedded specimens were first analyzed after staining with hematoxylin and eosin (FIG. 35). Liver from cortical cell groups showed various abnormalities such as deposition of fibrosis, and a large focus of inflammatory infiltration around the portal tract. Some animals showed extensive collapse and increased deposition of reticulum and development of extensive fibrosis in centrilobular areas and developed well differentiated HCCs with compressed hepatic parenchyma (FIG. 35). Liver from BEP neurons transplanted animals showed only mild fibrosis septae with almost normal histology (FIG. 35D).

TABLE 2
Tumor incidence in different groups.

| Group | | No. of | Tumor |
|---|---|---|---|
| No. | Group description | animals/group | Incidence* % |
| 1 | Cortical cells + Saline | 8 | 0% (8) |
| 2 | Cortical cells + DEN | 10 | 60% (10)^{a} |
| 3 | Stem cells + Saline | 8 | 0 (8) |
| 4 | Stem cells + DEN | 16 | 0% (16) |

^{a}p < 0.001, vs the rest as determined by the chi-square test.
*(Number of tumour-bearing rats/total number of rats in each group) × 100.

### BEP Neuronal Transplantation Prevents Hepatic Extracellular Matrix Accumulation

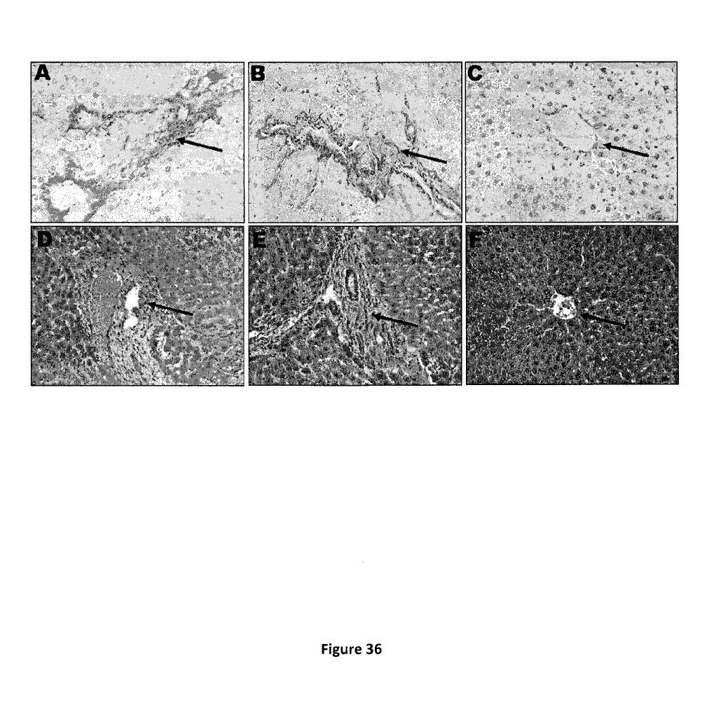

Fibrosis is a late event that has a deep impact on hepatocellular dysfunction during carcinogenesis. The appearance and accumulation of ECM (fibrosis) was analyzed in various groups by staining liver sections with Sirius red (FIG. 36 A-C) and Massson's trichrome staining (FIG. 36 D-F). Fibrosis was severe and surround at the centrilobular vein and creating a fine network of fibers around the groups of hepatocytes in the perivenular area in cortical cells implanted groups. Whereas, the accumulation of collagen was very less in the BEP neuron transplanted groups, and this showing that hepatic injury is prevented.

### BEP Neuronal Transplantation Prevents Development of Preneoplastic Lesions

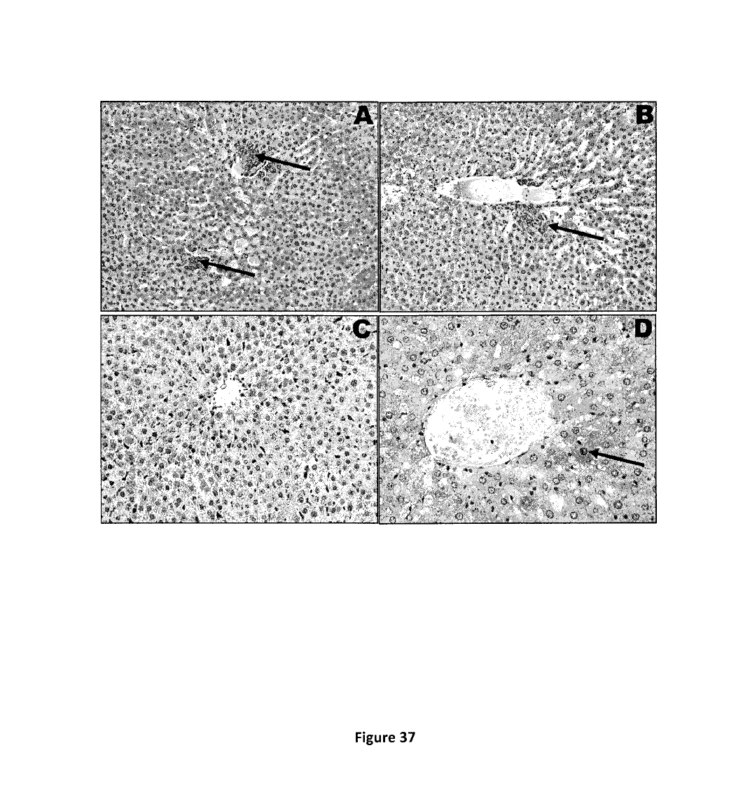

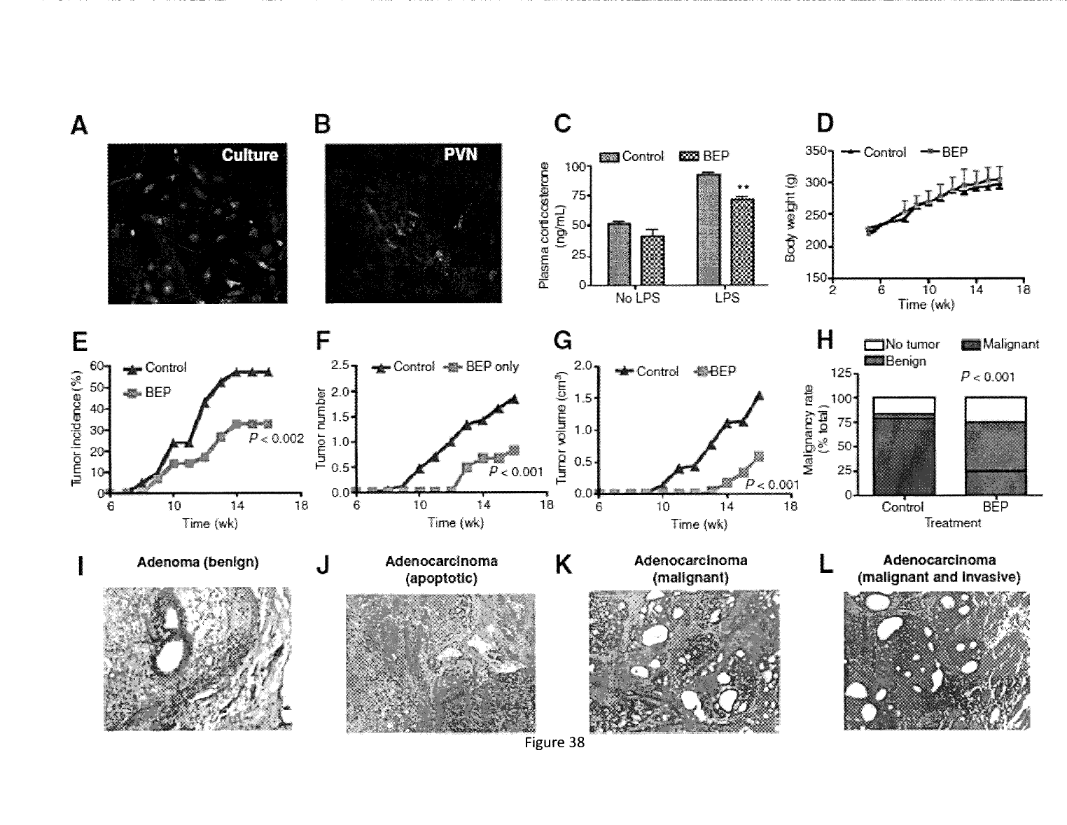

As expected, the liver from cortical cells implanted groups showed high number of GST-pi positive preneoplastic foci (FIG. 37) when compared to few small foci in BEP neuronal transplantation groups denotes that the formation of preneoplastic lesions was also blocked.

### Example 15

Breast cancer is the most frequent malignant disease among women. The National Cancer Institute estimated that there would be 40,170 deaths due to breast cancer (Smigal et al. (2006) Cancer J Clin 56:168-83), while the American Cancer Society predicted 192,370 new cases of invasive breast cancer among American women in the year 2009 (Cancer facts and figures 2007. American Cancer Society p. 9-10). Stress has been shown to be a tumor-promoting factor (Montgomery et al. (2010) J Adv Nurs 66:2372-90; Thaker et al. (2006) Nat Med 12:939-44; Thaker et al. (2008) Semin Cancer Biol 18:164-70; Reiche et al. (2004) Lancet Oncol 5:617-625; Marchetti et al. (1991) J Steroid Biochem Mol Biol 38:307-20). Emerging evidence suggests that chronic neurobehavioral stress can promote various tumor growth and progression secondary to sustained activation of sympathetic nervous system and inhibition of parasympathetic nervous system (Abo et al. (2002) Ther Apher 6:348-57; Webster et al. (2008) Cell Immunol., 252:16-26; Smyth et al. (2004) Immunol. Rev., 202:275-93). Stress can significantly affect many aspects of the body's immune systems. For example, higher levels of stress were shown to be associated with decrease in natural killer (NK) cell lysis activity, macrophage migration activity, decrease of T cell population, decreased lymphocyte proliferation following infection, and decrease in interferon-γ (IFN-γ) levels (reviewed in Webster et al. (2008) Cell Immunol., 252:16-26). These factors are reported to be important components of immunity against cancer (Smyth et al. (2004) Immunol Rev 202:275-93; Herberman, R. B. (1984) J Invest Dermatol 83:137-40). Therefore, manipulations to control the body's stress response may be beneficial to increase immunity and fight against cancer. β-Endorphin (BEP), an endogenous opioid polypeptide primarily produced by the hypothalamus and pituitary gland, is known to have the ability to inhibit stress hormone production, produce analgesia, and a feeling of well-being (Akil et al. (1984) Annu Rev Neurosci 7:223-55; Yermal et al. (2010) Biol Res Nurs 11:351-62). BEP is a cleavage product of proopiomelanocortin (POMC), which is also the precursor hormone for adrenocorticotrophic hormone and α-melanocyte-stimulating hormone (α-MSH). BEP neuronal cell bodies are primarily localized in the arcuate nuclei of the hypothalamus, and its terminals are distributed throughout the CNS, including the paraventricular nucleus (PVN) of the hypothalamus (Abo et al. (2002) Ther Apher 6:348-57). In the PVN these neurons innervate corticotropin releasing hormone (CRH) neurons and inhibits CRH release (Kawano et al. (2000) Neuroscience 98:555-65), while a μ-opioid receptor antagonist increases it (Plotsky et al. (1993) Ciba Found Symp 172:59-75). During stress, secretion of CRH and catecholamine stimulate secretion of hypothalamic BEP and other POMC-derived peptides, which in turn inhibit the activity of the stress system (Plotsky et al. (1993) Ciba Found Symp 172:59-75). BEP is known to bind to δ- and μ-opioid receptors and modulate the neurotransmission in sympathetic neurons via neuronal circuitry within the PVN to alter NK cell cytolytic functions in the spleen (Boyadjieva et al. (2006) Alcohol Clin Exp Res 30:1761-7; Boyadjieva et al. (2009) Alcohol Clin Exp Res 33:931-7). Abnormalities in BEP neuronal function are correlated with a higher incidence of cancers and infections in patients with schizophrenia, depression, and fetal alcohol syndrome and in obese patients (Lissoni et al. (1983) Br J Cancer 56:834-7; Bernstein et al. (2002) Cell Mol Biol 48:OL259-65; Zangen et al. (2002) Neuroscience 110:389-93; Pankov et al. (2002) Vopr Med Khim 48:121-30; Grinshpoon et al. (2005) Schizophr Res 73:333-41; Giovannucci et al. (2000) Gastroenterology 132:2208-25; Polanco et al. (2010) Alcohol Clin Exp Res 34:1879-87).

It has been shown that the neural stem cell-derived BEP neurons, when transplanted into the PVN, remained at the site of transplantation, decreased lipopolysaccaride (LPS)-induced levels of hypothalamic CRH and plasma corticosterone, increased NK cell cytolytic function and anti-inflammatory cytokines productions in response to immune challenge, and suppressed carcinogen-induced prostate cancer development in rats. It is not known whether BEP transplants prevent mammary tumor growth. Also, the effects of BEP neural activation on cancer progression and its metastasis to distant tissues are not evaluated. In this study, the effect of transplantation of in vitro differentiated BEP neurons from fetal neuronal stem cells into the hypothalamus on tumor incidence, growth, malignancy rate, and metastasis using a rat model of breast cancer was examined. In addition, immunologic and neurochemical changes pertinent to BEP action on tumor were determined.

### Materials and Methods

### Animals

Adult Sprague-Dawley and Fischer 344 male and female rats were purchased from Charles River and maintained in a controlled environment with a 12-hour light/dark cycle at Bartlett Hall Animal Research Facility. Male and female rats of each strain were bred and their fetuses or offspring were used in this study. All the animals were housed individually, allowed free access to regular rat chow, water, and maintained their normal physical activities throughout the study. Animal care was done in accordance with institutional guidelines and complied with NIH policy.

Preparation of BEP Cells from Neural Stem Cells

Neural stem cells were isolated from 17 days old fetal rat brains of Sprague-Dawley rats and then differentiated these cells into BEP neurons in culture to use in this study. cAMP and pituitary adenylate cyclase-activating polypeptide (PACAP) were used to differentiate BEP neurons from rat fetal neural stem cells. To control for transplantation, cortical cells prepared from 17-day-old fetal rat brains were used. Prior to transplantation, differentiated BEP cells were dissociated and resuspended at a concentration of 20,000 viable cells/mL in HEPES-buffered Dulbecco's modified Eagle's medium (DMEM)-containing serum supplement (30 nmol/L selenium, 20 nmol/L progesterone, 1 mmol/L iron-free human transferrin, 5 mmol/L insulin, 100 mmol/L putrescine, and antibiotics), cAMP (10 mmol/L), and PACAP (10 mmol/L) for the transplantation. Cells were placed on ice throughout the grafting session. Cell viability, assessed by the trypan blue exclusion assay, was routinely more than 90%. The composition of the differentiated cultures, with respect to the absence of undifferentiated neural stem cells and the presence of mature BEP-producing cells, was verified before grafting by staining for the immature neural marker nestin and/or vimentin, and for BEP using immunocytochemistry.

### Determining the Functionality of BEP Cells

The functionality of the transplanted cells was verified by doing a physiologic test in the transplanted animals followed by confirming the presence of BEP neurons at the site of transplantation after the termination of the experiments. Animals transplanted with BEP neurons have increased expression of POMC mRNA and decreased expression of CRH mRNA in the PVN and reduced response of plasma corticosterone following a LPS challenge. In this study, the function of transplanted BEP neurons was verified by determining the changes in corticosterone response to LPS. A 100 mg/kg dose of LPS was used for a period of 2 hours (which was found to be an effective dose; Sarkar et al. (2007) Endocrinology 148:2828-34) to determine the changes in the plasma corticosterone responses. After termination of the experiment, the brain was collected and processed for histochemical verification of the presence of transplanted BEP cells in the PVN of the hypothalamus using immunohistochemical methods. PVN nuclei do not contain BEP neuronal cell bodies in situ (control-transplanted PVN did not have any BEP staining), hence the immunocytochemically detected BEP cells in this area were considered transplanted cells. All the animals included in cancer study showed transplanted BEP cells in the PVN.

### Tumor Induction and Characterization

To determine the effect of BEP neuronal transplants on mammary tumor growth and progression, 50 days old ovary intact virgin Sprague-Dawley rats were injected intraperitoneally (i.p.) with a dose of NMU (50 mg/kg body weight). Six weeks after the NMU injection, animals were anesthetized and injected with cortical neurons (control) or BEP neurons in both sides of PVN of the hypothalamus using stereotactic procedures. No tumors were detected at this time. Beginning 1 week, when the animals recovered from the brain surgery, animals were weighed and palpated every week to check for tumor growth. Tumor length, width, and depth were measured with a calibrator. Sixteen weeks after the NMU injection, animals were sacrificed, tumors were collected, and slices of tumors were immersed in formalin and processed for histology staining. Fixed tissue was dehydrated, cleared, and paraffin infiltrated overnight using a tissue processor. The tissues were paraffin embedded, sectioned into 5-mm thick slices and placed on slides. One slide from each tissue was stained with hematoxylin and eosin (H&E) to evaluate tissue histology and tumor pathology. Slides were evaluated by a pathologist blinded to treatment. Ductal/cystic hyperplasia was defined by increased proliferation of benign glandular structures, with predominantly regular cells and nuclei. Adenomas were defined by a more solid phase glandular structure with regular cells and nuclei predominating. Adenocarcinomas presented primarily as solid-phase lesions containing many atypical and anaplastic cells, a high mitotic rate (including numerous atypical mitoses) and observable zones of tumor necrosis (apoptotic) and some show significant invasiveness.

### Immunohistochemical Localization of Various Proteins

Thin paraffin sections (5 mm) of mammary tumors were stained using the ABC Elite Vectastain Kit (Vector Labs) according to manufacturer's instructions using various primary antibodies. Primary antibodies for immunohistochemistry were used as follows: polyclonal rabbit antibodies against TNFα (1:250), NF-κB (1:100), E-cadherin (1:250), and N-cadherin (1:250; all from Abeam), as well as Snail (1:200), Slug (1:200), and Twist (all from Santa Cruz Biotechnology). After the primary antibody incubation and PBS wash, sections were incubated with peroxidase-coupled anti-rabbit Ig ImmPRESS reagent (Vector Laboratories, Inc.). Antigen localization was achieved by using the 3,3′-diaminobenzidine-peroxidase reaction and sections were dehydrated, and coverslipped. To evaluate the immunohistochemical staining, sections were photographed using Nikon-TE 2000 inverted microscope. Intensity of staining was categorized as negative (−) and strongly positive (+++).

### Western Blotting

For Western blotting, tumor tissue extracts equivalent to 50 μg total protein were separated by 4% to 20% SDS-PAGE and transferred overnight to immobilon-P polyvinylidene difluoride membranes. Membranes were incubated with primary antibody for 18 hours at 4° C. in blocking buffer. Membranes were then washed and incubated with peroxidase-conjugated secondary antibody (1:5,000) for 1 hour. Afterwards membranes were washed and then incubated with enhanced chemiluminescence Western blot chemiluminescence reagent (Pierce). Membranes were exposed to X-ray films and developed using X-Ray developer. Actin served as an internal control for loading and transfer of equal amounts protein samples. Details of all primary antibodies used are described above.

### Metastasis Study

MADB106 cells, a Fischer 344 rat mammary adenocarcinoma cell line maintained in DMEM containing 10% fetal calf serum, was used. Female Fischer-344 rats were transplanted with BEP cell transplants or control cell transplants at 50 days of age. After about 2 weeks, these PVN cell-transplanted rats were inoculated with MADB106 tumor cells (100,000 cells/0.2 mL/rat) into jugular vein under sodium Nembutal solution (50 mg/kg) anesthesia. Wounds were closed with a surgical clip, and rats were left on heating pad till recover. Rats were sacrificed at 4 weeks, after the tumor cells inoculation, whole body of each animal was inspected for the presence of visible tumors. Tumors were only located in lungs in most animals, and at the inoculation site in a few animals. Lungs were collected, fixed in formalin, embedded with paraffin, and sectioned for H&E staining. Stained slides were examined under the microscope for the verification of the presence of tumor. Brain tissues were processed for verification of the site and viability of BEP cell transplantation.

### Determination of Pharmacologic Modification of Autonomic Influence on Immune System on Lung Metastasis

It was hypothesized that the beneficial effect of BEP neuron transplants on immune function is regulated through autonomic nervous system, and that central BEP modulate peripheral immune function by inhibiting the sympathetic activity and activating the parasympathetic activity. Metaproterenol (MET) is an agonist for β-receptors, which are a class of G protein-coupled receptors that are targets of the catecholamines, especially norepinephrine and epinephrine (Dokur et al. (2004) J Neuroimmunol 5:148-57; Page et al. (2008) J Neuroimmunol 193:113-9). Methyllycaconitine (MLA) is a selective antagonist of α7 nicotine acetylcholine receptor (α7 nAChR; Wu et al. (2011) Clin Cancer Res 17:3533-41). By injecting MET and MLA i.p., the stimulation of sympathetic nerves and inhibition of parasympathetic nerves was mimicked. Naloxone, an opiate receptor antagonist, was used to test whether BEP neuronal effects is acted via opiodergic receptor (Boyadjieva et al. (2006) Alcohol Clin Exp Res 30:1761-7). Forty-eight BEP neuron-transplanted Fischer rats and 48 control rats were divided into 8 groups and treated with saline, naloxone (10 mg/kg body weight), MET (0.8 mg/kg body weight), or MLA (2.5 mg/kg body weight) for 8 days (the doses used for these drugs are recognized as effective biological doses). A day after the first injection of these agents, animals were inoculated with MADB106 cells (100,000/0.2 mL/rat). Twenty-four hours after tumor cell inoculation, blood was collected by orbital puncture. Plasma was collected, and peripheral blood mononuclear cells (PBMC) were separated for migration assay and NK cytolytic assay. These animals continued receiving the blocker treatments for 7 days. After 4 weeks they were sacrificed, their lungs were fixed in formalin and processed to H&E staining for determination of cancer pathology. Brain tissues were processed for verification of the site and viability of BEP cell transplantation.

Immune Reaction after Tumor Cell Inoculation

The immune reaction to tumor cell inoculation was checked in animals after they received BEP neuron transplants or cortical neuron transplants for 4 weeks. Animals were anesthetized with Nembutal, 1 mL of blood was drawn from jugular vein, and MADB106 cells (100,000/0.2 mL/rat) were inoculated into the jugular vein. After 24 hours, animals were anesthetized again. One milliliter of blood was drawn from the jugular vein, and then animals were sacrificed by decapitation. Vein blood before and after tumor inoculation was used for flow cytometry to determine cell populations in PBMC as described previously (Dokur et al. (2004) J Neuroimmunol 15:148-57). Trunk blood PBMCs and splenocytes were used for NK cell cytolytic assay and macrophage migration assay (CytoSelect 96-well cell migration assay, 5 mm, Cell BioLab). Trunk plasma was used for multicytokine assay. After termination of experiment, spleen were obtained and splenocytes were prepared and used for measuring mRNA levels of various cytokines and cytotoxic factors using real-time RTPCR methods as described previously (Dokur et al. (2004) J Neuroimmunol 15:148-57).

### Plasma Analysis of Hormones and Cytokines

Plasma was analyzed for corticosterone levels by a competitive ELISA (Immunodiagnostic Systems) according to manufacturer's recommendations. All samples were run on one 96-well plate for each variable. Plasma cytokine levels were measured by AssayGate, Inc. using their multiplex platform which measures multiple cytokines in a single plasma sample. Each sample was measured in triplicate. Immunerelated cytokines data are presented in figures.

### Statistics

Differences in average tumor incidence, tumor number, and tumor volume were assessed using 2-way ANOVA with a Bonferroni posttest at the level of α=0.05. To evaluate tumor type, a χ² test was done. Differences in tumor incidence and immune after various drugs were assessed using 1-way ANOVA with a Newman-Keuls post-hoc analysis at the level of α=0.05. t tests were used to evaluate the differences in various protein and cytokine levels.

### Results

BEP Neuronal Transplantation into the Hypothalamus Suppresses Mammary Tumor Growth and Progression

The in vitro produced BEP neuron was used in this study (FIG. 38A). Before testing BEP neuronal effects on mammary cancer growth, the functionality of BEP-neuron transplantation into the hypothalamus was tested following long-term transplants. For this, female rats at age of 50 days were implanted with BEP neuronal cells or control cells in both sides of PVN. After 2 to 3 months of cell transplants, these rats were inspected for changes in reproductive cyclicity and body growth. Like the untreated rats, both control and BEP cell-transplanted rats showed regular 4 to 5 days estrous cycle. Body weights of untreated control, control cell-transplanted rats and BEP cell-transplanted rats were similar (Untreated, 243±6; control cell transplanted, 251±5; BEP cell transplanted, 253±6; n=8-10). After 6 months, they were used to determine their plasma corticosterone response to LPS, as a functional test of BEP neuronal activity, and their brains were employed for immunohistochemical verification of transplanted neurons in the PVN. Transplanted BEP cells remained at the site of transplantation, as they were detected in the PVN by immunostaining (FIGS. 38A and B). As reported, BEP-transplanted animals showed lower plasma corticosterone response to LPS (FIG. 38C).

N-methyl-N-nitrosourea (NMU) was used to induce mammary cancer in rats. Six weeks after the administration of NMU, animals were anesthetized and injected with in vitro-differentiated BEP neurons or in situ cortical neurons, which served as the controls, in both sides of the PVN of the hypothalamus. Weekly body weight gain were similar between rats with control transplant or BEP transplants (FIG. 38D). Weekly measurement of tumor number, length and width for a period of 16 weeks revealed that BEP neurons implanted animals had lower tumor incidence, tumor number, and tumor volume (FIG. 38E-G). At the termination of the experiment, whole body inspection revealed that tumors were localized only in mammary glands. Tumors from the study were classified by histologic analysis (FIG. 38H-L). Histopathologic evaluation of tumors showed that, unlike control animals, which had mostly adenonocarinoma (both invasive and noninvasive), most of the BEP-treated animals had benign adenoma with glandular hypertrophy.

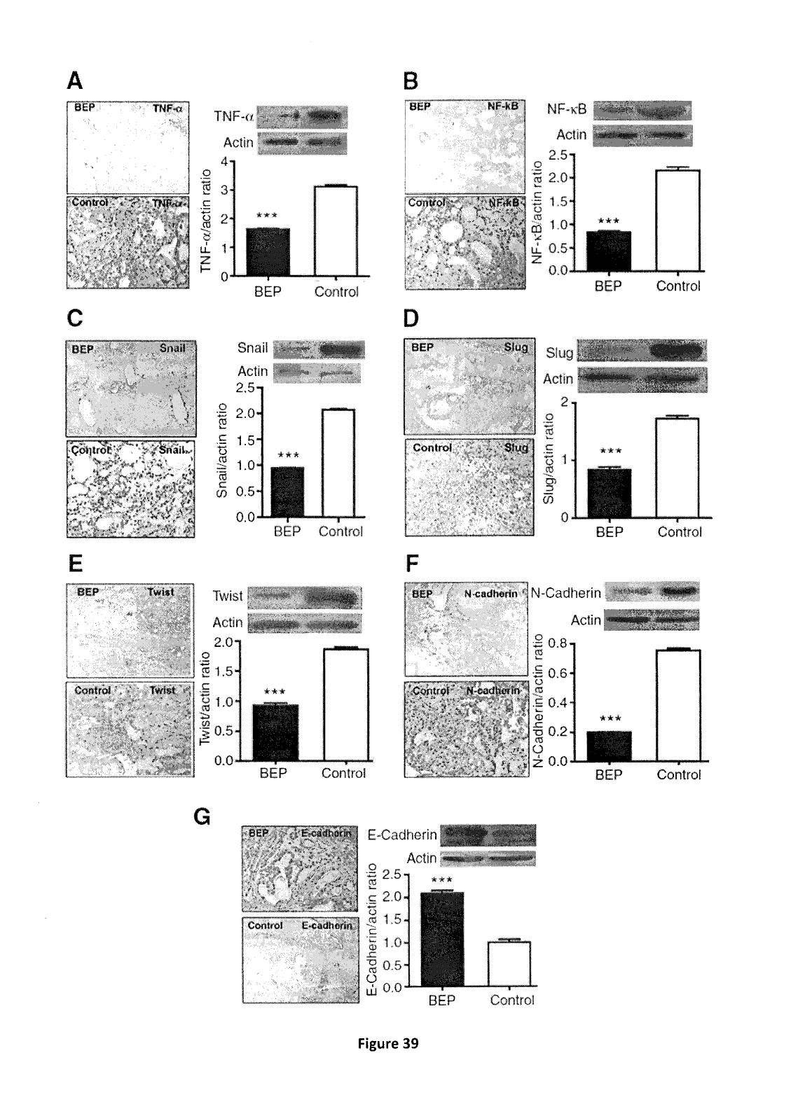

Numerous studies have shown that the inflammatory tumor microenvironment potentiates not only tumor development but also the progression of adenoma to carcinoma and induction, stabilization of epithelial-mesenchymal transition (EMT) in tumor tissues (Pensa et al. (2009) J Mammary Gland Biol Neoplasia 14,121-9; Wu et al. (2009) Cancer Cell 15:416-28; Yoshida et al. (2009) Med Mol Morphol 42:82-91). Loss of E-cadherin and aberrant N-cadherin expression associates with the acquisition of invasiveness and more advanced tumor stage for many cancers including breast cancer (de Herreros et al. (2010) J Mammary Gland Biol Neoplasia 15:135-47). Therefore, the expression of proinflammatory cytokine TNF-α and proinflammatory NFkB, transcription factors linked to the morphogenetic processes causing EMT (Snail, Slug, and Twist) expression, and expression of mesenchymal adhesion factor (N-cadherin) and epithelial adhesion factor (E-cadherin) were determined. Immunohistochemical and Western blot determination of various cytokines in tumor tissues obtained from rats with BEP cell transplants showed reduced levels of TNF-α and NF-kB, as well as Snail, Slug, and Twist with concomitant decreased in the level of N-cadherin, and increased the level of E-cadherin in mammary gland as compared with control-transplanted animals (FIG. 39A-G). In summary, the cellular and morphologic data of tumors indicate that BEP neuron transplants have prevented the development of advanced stage carcinoma possibly by suppressing the inflammatory response and repressing EMT factors in tumor tissues.

BEP Neuron Transplantation into the Hypothalamus Prevents Mammary Tumor Metastasis to Lung

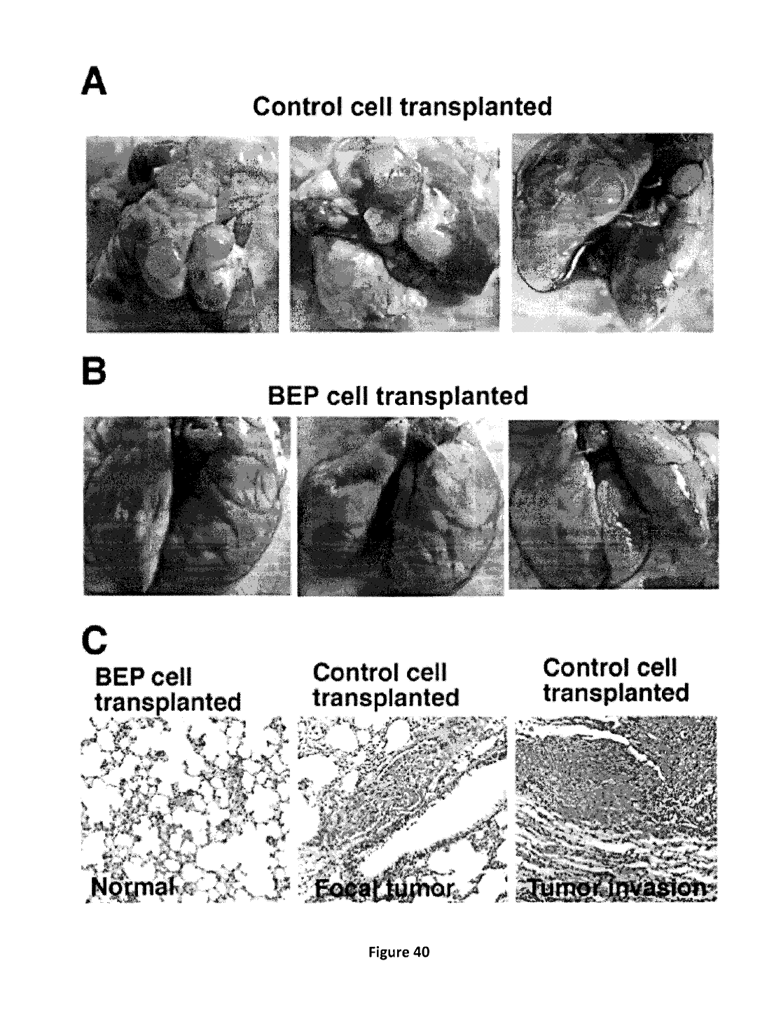

To determine the effects of BEP neuron transplants on tumor metastasis, nonimmunogenic syngeneic MADB106 mammary cancer cells were used, which are widely used for lung metastasis studies in rats (Ben-Eliyahu et al. (1996) Nat Med 2:457-60). Four weeks after inoculation of MADB106 cells through jugular vein, 70% to 80% of inoculated control cell-transplanted animals showed visible multiple tumor foci in lungs (FIG. 40A). Some of these animals also had a single visible tumor at the site where the tumor cells were inoculated. None of the BEP-transplanted animals showed visible tumors either in the lung or any other body sites. Histologic examinations of lung tissues identified focal and invasive tumors in control-transplanted rats while no tumors in BEP-transplanted rats (FIG. 40C). These data suggest that the BEP transplantation completely eliminated retention of MADB106 tumor cells in the lungs.

### Activation of Hypothalamic BEP Neurons Increases Innate Immune Functions

Because BEP transplantation increases immune activity in normal animals and since the innate immune system is critical for tumor cell clearance (reviewed in Jaiswal et al. (2010) Trends Immunol 31:212-9; Yang et al. (2006) Immunol Res 36:13-25), it was hypothesized that the elimination of tumor cells from lungs of BEP-transplanted animals may have been caused by increased innate immune function. Using the MADB106 cancer model, immune response (Hazlett et al. (2010) Ocul Immunol Inflamm 18:237-43) was checked 24 hours following tumor cell inoculation. Several studies have shown that MADB106 tumor cells metastasize only to the lungs and subjected to NK cell cytotoxic activity when injected intravenously (Ben-Eliyahu et al. (1996) Nat Med 2:457-60).

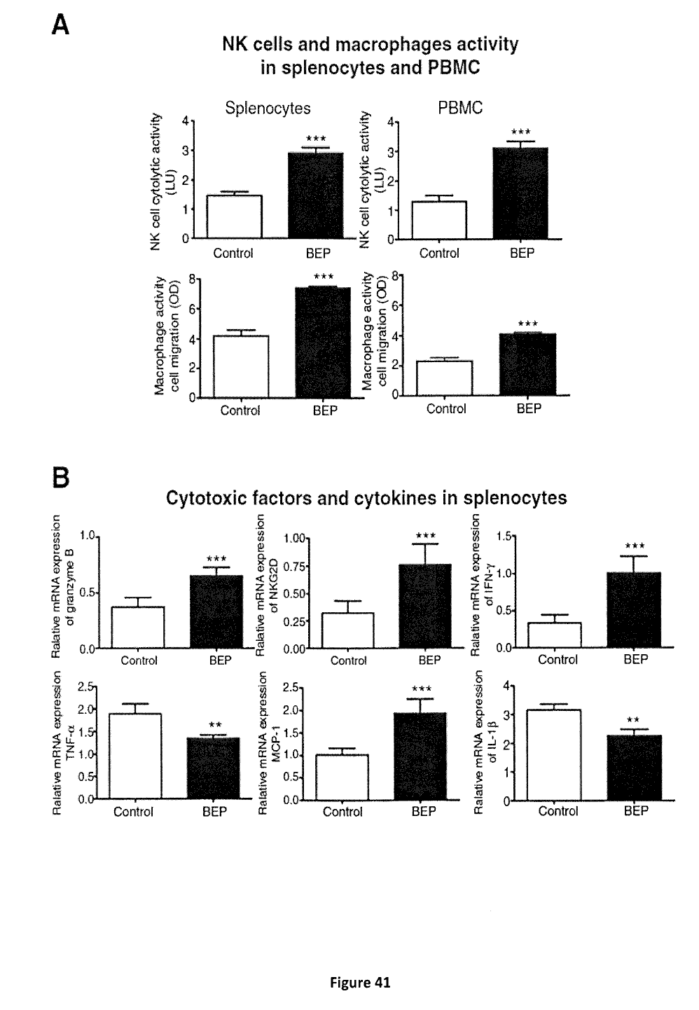

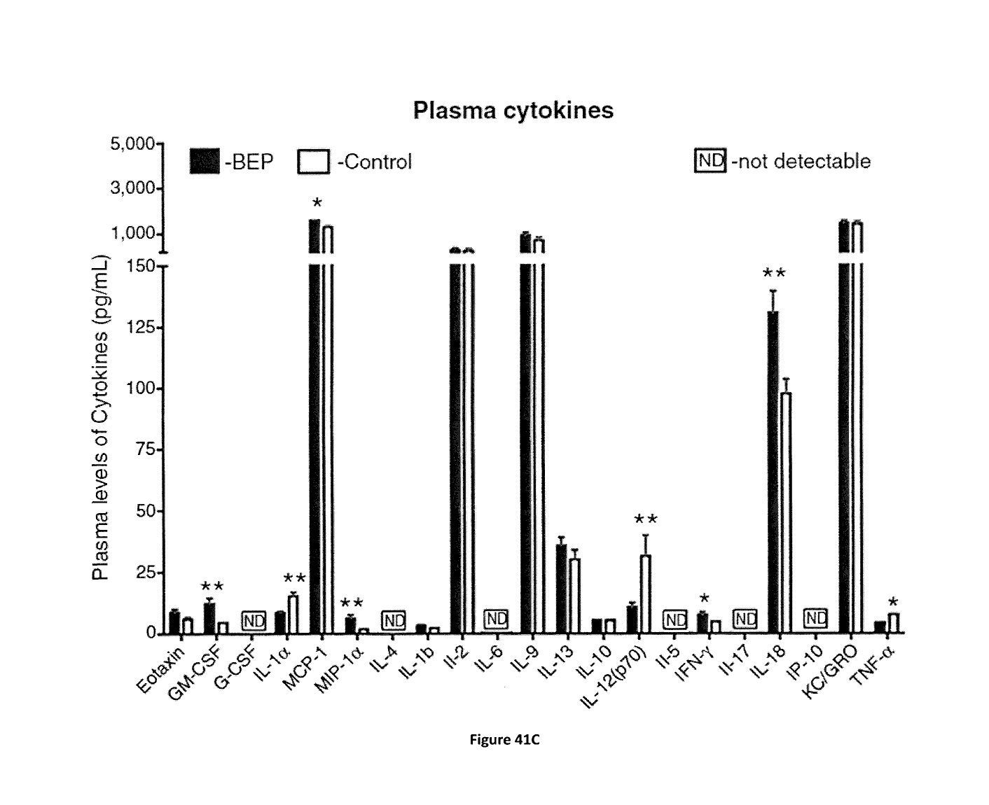

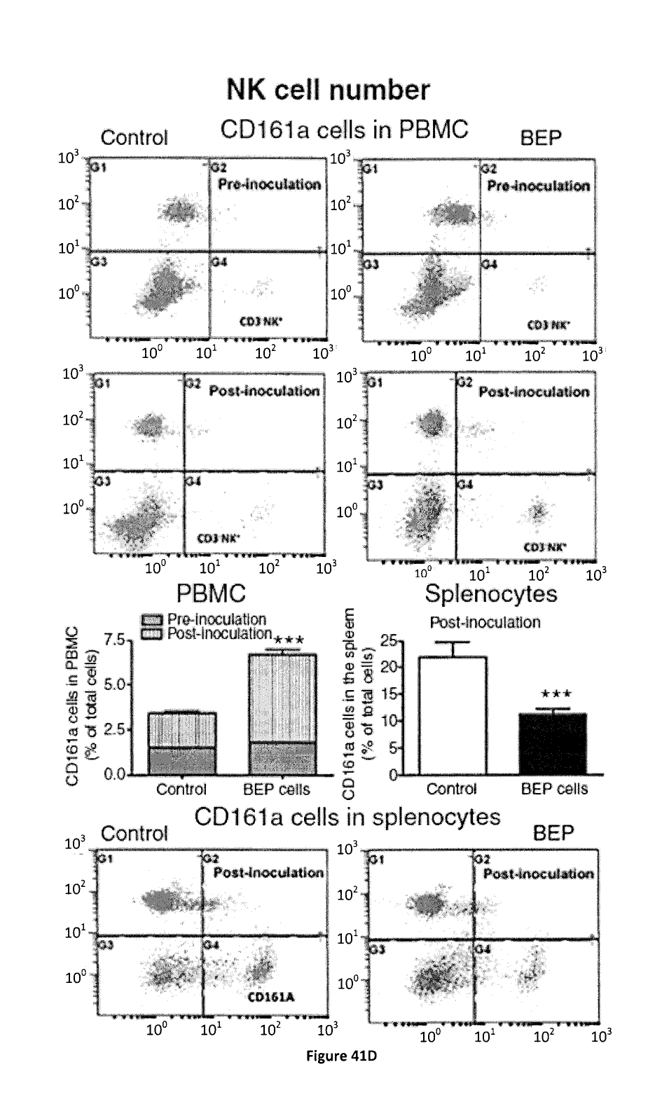

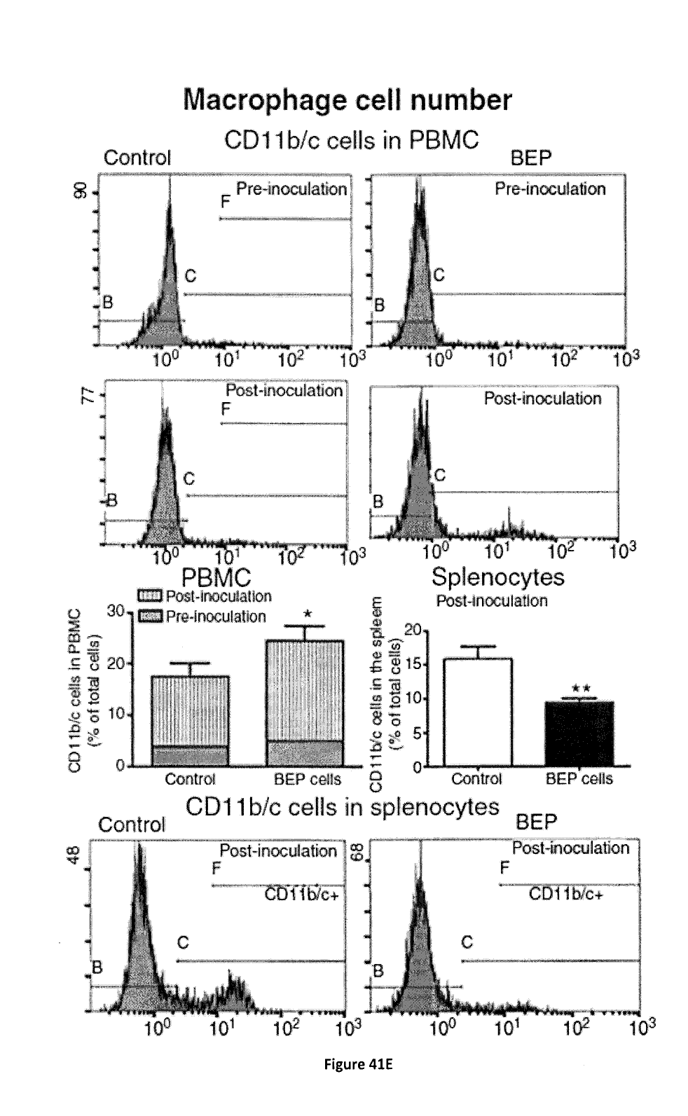

It was found that the BEP-transplanted rats had greater NK cell cytolytic activity in PBMCs and in splenocytes as well as greater macrophage migration and cell proliferation activity than those in control cell-transplanted rats (FIG. 41A). Consistent with these data, it was found that splenocytes of BEP-transplanted rats had higher mRNA and protein levels of NK cell cytolytic activity regulatory factors (e.g., granzyme B; Young et al. (1990) Annu Rev Med 41:45-54), NK cell activator receptor (e.g., NKG2; O'Connor et al. (2006) Immunology 117:1-10), cytokines that is produced after NK activation (e.g., IFN-γ, but lower TNF-α; Cox et al. (2000) Mol Biotechnol 15:147-54), a chemokine that recruits and activates macrophages (MCP1; Kyriakides et al. (2004) Am J Pathol 165:2157-66), and a protein that is produced in an increased amount from activated macrophages (IL-1β; Kaler et al. (2009) Oncogene 28:3892-902; FIG. 41B). Furthermore, measurement of cytokines and protein levels in PBMCs of tumor-inoculated rats showed that the levels of macrophage-regulatory/derived factors (GM-CSF, MIP-1α, IL-18; Miyazaki et al. (2000) Ann NY Acad Sci 902:342-6; Gregory et al. (2006) J Immunol 177:8072-9; Li et al. (2009) J Reprod Immunol 83:101-5) and an NK cell-regulatory/derived factor (IFN-γ; Cox et al. (2000) Mol Biotechnol 15:147-54) are higher in BEP-transplanted rats than those in control-transplanted rats (FIG. 41C). Also, lower levels of several inflammatory cytokines (IL-1α, IL-12, TNF-α; Patel et al. (2005) J Neuroinflammation 2:9) were observed in plasma of BEP-transplanted rats as compared with PBMCs of control-transplanted rats. In addition, BEP transplants increased NK and macrophage cell numbers in PBMCs, but decreased their numbers in splenocytes after tumor cell inoculations (FIGS. 41D and E), suggesting that the transplants also promoted migration of these immune cells out of the spleen to the blood and/or inhibited emigration from the blood into the spleen to promote defense against tumor cells. These data indicate that the BEP neuronal supplement promotes innate immunity and produces an anti-inflammatory environment in recipient animals.

### Antagonists of Opiate Receptor and α7 Nicotine Acetylcholine Receptors and Agonists of β-Adrenergic Receptors Prevent BEP Neuronal Ability to Enhance Immunity and Suppress Tumor Metastasis

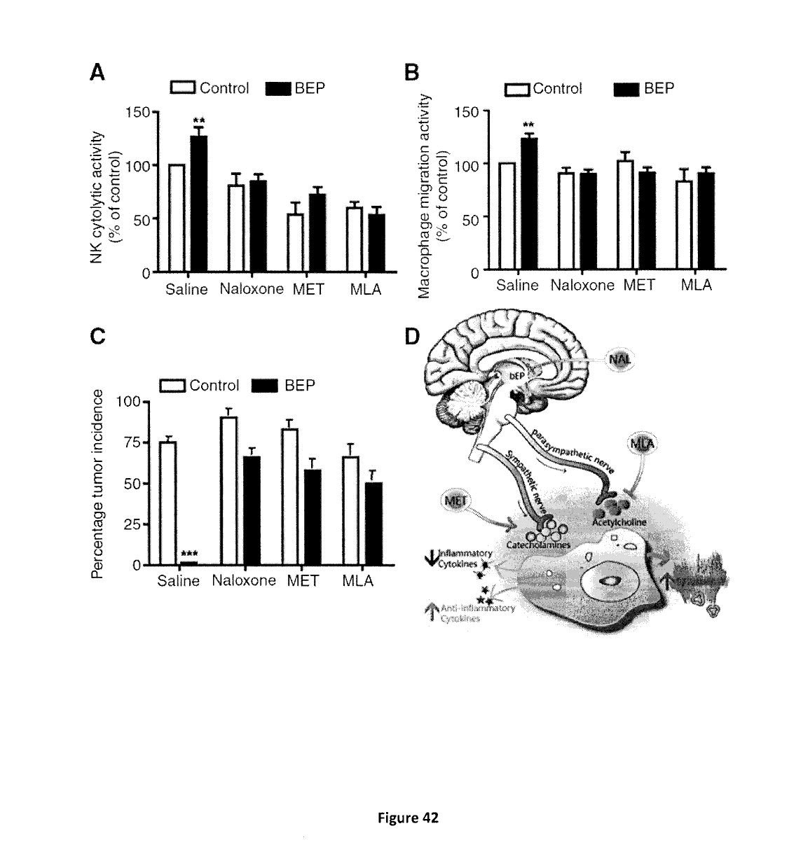

BEP neurons produce opioid peptides and other products of POMC (Imura et al. (1983) J Endocrinol Invest 6:139-49). To address the question whether BEP alone and/or other peptide products from the transplanted neurons are responsible for the observed actions on the immune system and cancer, the ability of a general opiate antagonist naloxone to block the effects of BEP neuron transplants on immune activation and metastasis prevention was tested. In addition, the effects of norepinephrine agonist MET and a α7 nicotine acetylcholine receptor (α7 nAChR), antagonist MLA was tested. It was found that 24 hours after tumor inoculation, PBMC NK cell activity (FIG. 42A) and macrophage migration activity (FIG. 42B) were higher in saline-treated BEP-transplanted animals than in saline-treated cortical cells-transplanted controls. Naloxone, MET, and MLA all had moderate or strong inhibitory effect on basal and BEP-stimulated NK cell activity and macrophage migration activity. Consistent with these findings, it was observed that naloxone, MET, and MLA prevented, at various degrees, the beneficial effect of BEP in eliminating tumor cell lung retention (FIG. 42C). These data indicate that BEP neurons activate innate immunity for cancer cell clearance via altering the function of the autonomic nervous system.

### Discussion

In situ, BEP neurons originating from the arcuate nucleus terminate in the PVN and are known to regulate both stress axis and immune functions. The data presented here show that the neural stem cell-derived BEP neurons, when transplanted in the PVN, remained viable and increased endogenous opioid inhibitory tone to the HPA axis so that plasma corticosterone levels responded lower during stressful conditions like immune challenge. BEP cell transplantation does not affect general body growth or reproductive hormone profiles as the body weight and reproductive cyclicity of these rats were similar to those in controls. Within the context of immune-related function, in situ BEP neurons in the hypothalamus are known to increase NK cell function via inhibition of sympathetic neurons to the spleen; the present data are consistent with this (FIG. 42D). Furthermore, it was observed for the first time that BEP neurons were able to stimulate parasympathetic neurons to activate both NK cells and macrophages. Recently, a role for parasympathetic neurons in immune activation has been revealed (Li et al. (2009) J Reprod Immunol 83:101-5). Studies have shown that decreased NK cells activity is associated with growth and progression of variety of cancers in animals (Gorelik et al. (1982) Int J Cancer 30:107-12; Talmadge et al. (1980) Nature 284:622-4) and humans (Natascha et al. (2007) EXCLI J 6:1-9; Fulton et al. (1984) Breast Cancer Res Treat 4:109-16; Garner et al. (1983) J Surg Oncol 24:64-6), because NK-cells represent a first line of defense against the metastatic spread of tumor cells (Gorelik et al. (1982) Int J Cancer 30:107-12). In breast cancer patients, low NK cell activity seems to be heavily related with larger tumor growth (Fulton et al. (1984) Breast Cancer Res Treat 4:109-16) and also a predictive parameter of advanced disease (stages II, III, and IV) than in women with limited disease (stage I; Garner et al. (1983) J Surg Oncol 24:64-6). At present, modulation of immune function especially enhancing NK cell activities, seems to be the most promising and new approach to cancer treatment. In this study, it was shown here that supplementation of BEP neurons, through transplants, prevents mammary tumor growth, progression, and metastasis. Importantly, when the BEP transplants were given at the early stage of tumor development, many tumors were destroyed possibly due to increased innate immune activity, and the surviving tumors lost their ability to progress to high-grade cancer due to BEP cells' suppressive effects on inflammation-induced EMT regulators.

It is well known that inflammatory tumor microenvironment propel the migration and invasion of tumor cells through induction of EMT (Wu et al. (2009) Cancer Cell 15:416-428). Hence, regulating inflammation and EMT may be a potential novel approach to reverse the progression of tumor. Another remarkable effect of the BEP transplantation was that it promoted the activation of the innate immune activity following tumor cell invasion to such an extent that tumor cell migration to another site was completely halted. The NK cells and macrophages are critical components of the innate immune system and play a vital role in host defense against tumor cells (Wakimoto et al. (2003) Gene Ther 10:983-90). Hence, the increased level of innate immunity may have caused unfavorable conditions for cancer cell survival. In the BEP cell-treated animals the lower inflammatory milieu that was achieved by the higher level of anti-inflammatory cytokines and the lower level of inflammatory cytokines may have also been involved in inhibiting cancer growth and transformation. Several studies have addressed the involvement and roles of the inflammatory chemokines and cytokines in breast malignance (Pensa et al. (2009) J Mammary Gland Biol Neoplasia 14, 121-9; Wu et al. (2009) Cancer Cell 15:416-28). In addition, pharmacologic modification of autonomic function significantly blocked the innate immune response and enhances the tumor cell metastasis and thus provides a plausible molecular mechanism for the protective role of BEP neurons against the cancer progression and metastatic diffusion of mammary tumor cells. This study not only identified the importance of stress maintenance in regulating immune function in cancer patients but also provided support for a therapeutic use of BEP cell therapy for controlling breast cancer and other cancers.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of inhibiting cancer in a subject in need thereof, said method comprising administering at least one agent which increases β-endorphin neurons in the subject, thereby inhibiting said cancer.

2. The method of claim 1, wherein said cancer is selected from the group consisting of prostate cancer, lung cancer, liver cancer, and breast cancer.

3. The method of claim 2, wherein said cancer is prostate cancer or breast cancer.

4. The method of claim 1, wherein said cancer is metastatic cancer.

5. The method of claim 1, wherein said method comprises administering at least one cyclic adenosine monophosphate (cAMP) or a cAMP analog to said subject.

6. The method of claim 5, wherein said cAMP analog is pituitary adenylate cyclase activating peptide (PACAP) or dibutyryl cyclic adenylate cyclase (dbcAMP).

7. The method of claim 5, wherein said cAMP or analog thereof is administered via a slow release device.

8. The method of claim 7, wherein said cAMP or analog thereof is administered to the subject in nanospheres.

9. The method of claim 1, wherein said method comprises administering β-endorphin neurons to the subject.

10. The method of claim 9, wherein said β-endorphin neurons are generated by incubating neuronal stem cells with at least one cAMP or a cAMP analog.

11. The method of claim 10, wherein said cAMP analog is pituitary adenylate cyclase activating peptide (PACAP) or dibutyryl cyclic adenylate cyclase (dbcAMP).

12. The method of claim 1, wherein the agent is administered into the brain.

13. The method of claim 12, wherein the agent is administered into the hypothalamus.

14. The method of claim 13, wherein the agent is administered into the third ventral.

15. The method of claim 1, further comprising the administration of at least one other chemotherapeutic agent.

16. The method of claim 1, further comprising the administration of radiation therapy.

17. A method of inhibiting an immunological disease or inflammatory disease in a subject in need thereof, said method comprising administering at least one agent which increases β-endorphin neurons in the subject, thereby inhibiting said immunological disease or inflammatory disease.

18. The method of claim 17, wherein said immunological disease or inflammatory disease is selected from the group consisting of rheumatoid arthritis, diabetes, thyroid disorder, celiac disease, obesity, inflammatory bowel syndrome, and lupus.

19. A method of inhibiting stress in a subject in need thereof, said method comprising administering at least one agent which increases β-endorphin neurons in the subject, thereby inhibiting said stress.
